(19) United States
(12) Patent Application Publication
(10) Pub. No.: US 2011/0021429 A1
(43) Pub. Date: Jan. 27, 2011

(54) Renoprotection by growth hormone-releasing hormone and agonists

(76) Inventors: Pierrette Gaudreau, Brossard (CA); Karyne Theriault, Montreal (CA); Julie Bedard, Carignan (CA); Chantale Boisvert, Ville St-Laurent (CA)

Correspondence Address: BCF LLP, 1100 RENE'-LE'VESQUE BLVD. WEST, 25TH FLOOR, MONTREAL, QC, H3B-5C9, CA

(21) Appl. No.: 12/285,282

(22) Filed: Oct. 1, 2008

Related U.S. Application Data
(60) Provisional application No. 60/960,477, filed on Oct. 1, 2007.
(60) Provisional application No. 61/006,057, filed on Dec. 17, 2007.

(51) Int. Cl. A61K 38/25; A61K 38/16; A61P 13/12; C07K 14/00; C07K 16/00; C07H 21/04; C12N 15/63; C12N 5/00; G01N 33/53

(52) U.S. Cl. 514/11.2; 514/15.4; 530/350; 530/387.9; 536/23.1; 435/320.1; 435/325; 436/501

(57) ABSTRACT

The present invention relates to renoprotection by growth hormone-releasing hormone and agonists. More specifically, the present invention relates to methods for protecting a mammal against oxidative renal damage, of promoting regeneration of kidney cells in a mammal in need thereof and/or of preventing the death of kidney cells due to oxidative stress. The present invention also relates to the identification of rat and human renal GHRH-R sequences.

### CROSS-REFERENCE TO RELATED APPLICATIONS

This patent application claims the benefit of U.S. Provisional patent application Ser. No. 60/960,477 filed Oct. 1, 2007 and Ser. No. 61/006,057 filed Dec. 17, 2007 the disclosure of which are herein incorporated by reference in their entirety.

### FIELD OF INVENTION

The present invention relates to the field of renoprotection by growth hormone-releasing hormone and agonists.

### BACKGROUND OF THE INVENTION

The pituitary growth hormone-releasing hormone receptor (GHRH-R) has been cloned in several mammalian species,¹⁻⁴ including normal human pituitary^{2, 5, 6} and adenomas.⁵⁻⁷ More recently, GHRH-R was reported in avian⁸ and fish pituitary.⁹ The rat pituitary contains a major GHRH-R mRNA transcript (2.5 kb) and a less abundant one (4 kb; ≈20% of the 2.5-kb in 2-month-old rats).^{2, 10} While the 2.5-kb transcript generates the 423 amino acid functional GHRH-R,¹¹ the role and structure of the 4-kb transcript remain to be elucidated. The 47-kDa-encoded rat protein belongs to the subfamily B-III of G protein-coupled receptors, which also include receptors for VIP, secretin, glucagon, GIP, PTH, calcitonin, CRF and PACAP.² In somatotrophs, the specific binding of hypothalamic GHRH to functional plasma membrane receptor represents the primary event leading to GH secretion¹²⁻¹³ and synthesis¹² mainly through an adenylate cyclase/cAMP/protein kinase (PK) A pathway¹⁴⁻¹⁷ and possibly a PKC pathway.¹⁸ GHRH-mediated GHRH-R activation is also involved in somatotroph proliferation and differentiation via PKA^{2, 19-22} and mitogen-activated protein (MAP) kinase pathways.²³⁻²⁴

Apart from the anterior pituitary, a GHRH-GHRH-R system has been identified in rat brain, spleen and thymus, ovary, placenta, testis and renal medulla. Intrasuprachiasmatic/medial preoptic area administration of GHRH stimulates dietary protein intake in free-feeding rats²⁵ while it promotes sleep in the intrapreoptic region.^{26 In rat spleen and thymus, a functional GHRH-GH axis was shown to mediate lymphocyte proliferation through a GHRH-induced GH mechanism.}²⁷ In human and rat reproductive systems, the presence of GHRH-R mRNA² and immunoreactivity²⁸ has been reported as well as GHRH-mediated effects on regulation of sex steroid levels,²⁹ granulosa cell differentiation,³⁰ placental growth,³¹ and gonadotropin stimulation of testosterone.³²

A functional GHRH-R has been identified in the rat renal medulla.^{33, 34} Boulanger et al. demonstrated the presence of specific, reversible and saturable binding for [¹²⁵]-Tyr¹⁰]hGHRH(1-44)NH₂ in this tissue.³⁴ Moreover, stimulation of semi-purified Henle's loop (HL) cells with GHRH was shown to mediate GHRH-R internalization and regulation of its expression.³³ The highest level of renal GHRH-R mRNA was localized in HL by ribonuclease protection assay and in situ hybridization.³³ Its localization in HL and the tissue-selective regulation of pituitary and renal GHRH-R mRNA levels and its regulation during development and aging may suggests roles of GHRH-R in the renal medulla.³³

The present description refers to a number of documents, the content of which is herein incorporated by reference in their entirety.

### SUMMARY OF THE INVENTION

In one aspect thereof, the present invention relates to a method for protecting and/or treating a mammal against oxidative renal damage. The method may comprise the step of administering an effective amount of a ligand to (of the) GHRH renal receptor to the mammal.

In another aspect, the present invention relates to a method for preventing (lowering, inhibiting) the death of kidney cells (and/or loss of kidney cell function) due to oxidative stress in a mammal in need thereof. The method may comprise administering to the mammal a ligand to GHRH renal receptor.

In a further aspect, the present invention relates to a method of promoting regeneration of kidney cells and/or function in a mammal in need thereof. The method may comprise administering a ligand to the GHRH renal receptor the mammal.

In yet a further aspect thereof, the present invention relates to the new polypeptidic sequence of rat and/or human GHRH receptors, antibody that may bind same, nucleic acids that may encode same, a cell expressing same and/or vectors that may comprise the nucleic acid of the present invention.

In another aspect, the present invention relates to an assay for identifying a ligand capable of specific binding to the new rat and/or to the new human GHRH receptors and not to pituitary GHRH receptor. This assay may comprise contacting a test ligand with the polypeptide, measuring binding of the test ligand to the polypeptide and/or determining the identity of the test ligand.

In a further aspect, the present invention relates to a method for the diagnosis of renal oxidative stress and/or damage and diagnostic kits.

Further scope, applicability and advantages of the present invention will become apparent from the non-restrictive detailed description given hereinafter. It should be understood, however, that this detailed description, while indicating exemplary embodiments of the invention is given by way of example only, with reference to the accompanying drawings.

### BRIEF DESCRIPTION OF THE DRAWINGS

In the appended drawings which illustrates non-limitative exemplary embodiments of the present invention,

FIG. 1 GHRH-R mRNA levels in the renal medulla and purified thin and thick HL cells from 2-month-old healthy rats. Five (thin HL) and 20 μg (thick HL and medulla) of total RNA were analyzed by RPA. Results were expressed as per 20 μg of total RNA, in percentage of relative density to that obtained in 20 μg total RNA samples from the medulla. Results represent the mean±SEM of samples analyzed in duplicate from 2 independent RPA experiments and were normalized with both GAPDH and the cRNA external standard.**P<0.01 when compared to GHRH-R mRNA levels in total medulla (Dunnett's test);

FIG. 2 Immunocytochemical localization of the GHRH-R in purified thin limb cells from 2-month-old healthy rats. Co-localization of GHRH-R immunofluorescence (b, e) was assessed in renal cells from 2-month-old male rats using an anti-aquaporin-1 antibody, as a marker of descending thin limb cells (a) and an anti-CIC-K antibody, as a marker of ascending thin limb cells (d). Immunolabeling was specific and no labeling was observed when substituting the anti-GHRH-R(392-404) Ab for normal IgGs (data not shown). Nuclei were labeled with DAPI. Results are representative of three independent experiments;

FIG. 3 Visualization of immunoreactive GHRH and CIC-K1 chloride channel and PCR amplification of preproGHRH in purified thin limb cells from 2 month-old healthy rats. A) Labeling of GHRH (a, b) and the CIC-K1 chloride channel (c, d) was performed in purified thin limb cells using an anti-rat GHRH(1-43)OH Ab and an anti-CIC-K Ab. Overlay of GHRH and CIC-K immunofluorescence is shown in (f). The specificity of labeling was assessed by substituting the anti-rat GHRH(1-43)OH Ab for normal IgGs (e). B) Representative agarose gel electrophoresis of preproGHRH and GAPDH PCR products and molecular weight markers.

FIG. 4 Effect of a 2-, 7- or 14-day 8%-NaCl dietary intake on medullary GHRH-R mRNA levels from 2-months-old rats. A) Autoradiographic representation of GHRH-R mRNA, GAPDH mRNA and RPR-64 Msc I cRNA external standard (40 pg) signals analyzed by RPA, from rats fed 8%- or 0.3%-NaCl (control) diet. B) GHRH-R mRNA levels expressed per 20 μg total RNA. For the 2-day experiment, 5-6 individual rats were used in each group for both RPA and statistical analysis, while for the 7- and 14-day experiment, 7-8 individual rats were used. Results are expressed in percentage of relative density to that obtained in the medulla from control rats and represent the mean±SEM of individual samples from each group, analyzed in triplicate twice and normalized with GAPDH and the cRNA external standard. *P<0.05 and **P<0.01 when compared to GHRH-R mRNA levels in the medulla from control rats (Student's t test);

FIG. 5 Effects of a 2-, 7- or 14-day 8%-NaCl dietary intake and a 3-day water deprivation on anterior pituitary GHRH-R mRNA levels from 2-months-old rats. A-D) GHRH-R mRNA levels analyzed by Northern blotting and expressed per 12 μg total RNA. For the 2- (A), 7- (B) and 14-day (C) 8%-NaCl experiment, 7-8 individual rats were used in each group for both Northern blotting and statistical analysis, while for the 3-day water deprivation (D), 3 (controls) and 7 (deprived) individual rats were used. Results are expressed in percentage of relative density to that obtained in the pituitary from control rats and represent the mean±SEM of individual samples from each group, analyzed in duplicate and normalized with normalized with rRNA 28S. *P<0.05 and ***P<0.001 when compared to GHRH-R mRNA levels in the pituitary from control rats (Student's t test);

FIG. 6 Basal and GHRH-stimulated cAMP levels in semi-purified thin limb cells from 2-month-old rats, following a 2-, 7- or 14-day 8%-NaCl dietary intake. Basal and net GHRH-stimulated cAMP levels were quantified by EIA (fmol/μg prot) in freshly dispersed semi-purified thin limb cells of rats fed 2- (A), 7- (B) or 14- (C) days a 8%- or 0.3%-NaCl (control) diet. Results are expressed in percentage of control values both for basal and stimulated cAMP levels. Cells from 4 individual rats were used in each diet and control group. *P<0.05 and **P<0.01 when compared to cAMP levels in semi-purified thin limb cells from control rats (Student's t test);

FIG. 7 Effect of a GHRH in vivo sc administration of GHRH in 2 month-old rats fed a 8%- or 0.3%-NaCl diet on anti-BrdU labeling. BrdU was injected ip 2 h prior sacrifice (100 mg/hg BW). Rats were fed a 8%- or 0.3%-NaCl (control) diet and concurrently injected with rGHRH(1-29)NH₂ (1 mg/kg BW). Purified thin limb cells were cultured 16 h on coverslips and processed for immunocytochemistry. A) Increased number of cells exhibiting specific anti-BrdU labeling, colocalizing either with DAPI (nuclear) or Mitotracker red CMXRos (mitochondrial) and B) increased anti-BrdU total fluorescence intensity in nuclear or mitochondrial compartment were expressed in percentage of control values (0.3%-NaCL salt diet, GHRH vehicle injection).*P<0.05 when compared to levels in purified thin limb cells from control rats (Dunnett's t test);

FIG. 8 Effect of a GHRH in vivo sc administration in 2 months-old rats on the regulation of GHRH-R and CIC-K1 mRNA levels in purified thin limb cells. Two-month-old healthy male Sprague Dawley rats, received a subcutaneous administration of rGHRH(1-29)NH₂ (1 mg/kg BW/day) or the saline vehicle for 2 days. (A) GHRH-R and CIC-K1 (B) mRNA levels were analyzed by real-time RT-PCR. Eight animals were used in control and treatment. Group 1=3 rats. Group 2=5 rats. *P<0.05 and **P<0.01 when compared to levels in purified thin limb cells from control rats (Dunnett's t test) (C) Regulation of GHRH-R mRNA levels in thin limb cells of 2-month-old healthy male Sprague Dawley rats was also studied following a subcutaneous administration of rGHRH(1-29)NH2 (0.5 mg/kg BW/day) or the saline vehicle, for 2 days;

FIG. 9 rGHRH(1-29)NH₂-induced cell proliferation in semi-purified thin limb cells. Proliferation was assessed after a 60-h cell culture period, using a CellTiter 96R Aqueous one solution cell proliferation assay. Results represent the mean±SEM of 2 independent experiments performed in duplicate. *P<0.05, **P<0.01 when compared to control levels (Dunnett's t test);

FIG. 10 RT-PCR products from rat and porcine renal medulla and anterior pituitary obtained with a panel of primers of the pituitary GHRH-R. A) Rat and B) porcine total RNA was used. Lanes 1, 2: sense and antisense 5′ end primers, lanes 3, 4: sense and antisense middle portion and lanes 5, 6: sense and antisense 3′ end primers, respectively;

FIG. 11 GHRH-R 5′- and 3′-RACE products from rat renal medulla and anterior pituitary Poly(A)⁺RNA. A, Renal medulla (lane 1), anterior pituitary (lane 2) and liver (lane 3, negative control) 5′ cDNA products were obtained using the primer pair NUP/Ex-9/10P (Table 2). Each product from the renal medulla and anterior pituitary was isolated by gel extraction, (renal medulla: lanes 4-6; pituitary: lane 7). B, Renal medulla (lane 1), anterior pituitary (lane 2) and liver (lane 3) 3′ cDNA products were obtained using the primer pair NUP/N-Ex7/8 (Table 2). Each product from the renal medulla and anterior pituitary was isolated by gel extraction, (renal medulla: lanes 4-5; pituitary: lanes 6-7). Data are representative of three independent experiments;

FIG. 12 Schematic genomic organization of rat GHRH-Rs. Exons organization and genomic location of anterior pituitary and renal medulla GHRH-R on chromosome 4. Numbers correspond to GHRH-R exons. The first and last nucleotides were at position 18576 and 51026 on AC091710 sequence, respectively, while those of the pituitary GHRH-R were in position 38055 and 51026;

FIG. 13 is the nucleic acid sequence of the rat renal medulla GHRH receptor;

FIG. 14 is the deduced protein sequence in the rat renal medulla: 371 aa, the 52 first N-terminal amino acid segment is absent;

FIG. 15 illustrates a comparison of the rat GHRH-R protein sequence between anterior pituitary and the renal medulla;

FIG. 16 RT-PCR validation of renal medulla GHRH-R variants in rat thin limb Henle's loop cells and anterior pituitary total RNA. A, tlHL cells (lanes 1, 4), anterior pituitary (lanes 2, 5) and liver (lanes 3, 6) GHRH-R cDNA products using primers pairs Ex-1M/Ex-11M (lanes 1-3) and Ex-2M/Ex-11M (lanes 4,-7). B, tlHL cells (lane 1), anterior pituitary (lane 2) and liver (lane 3) GHRH-R cDNA products using the primer pair Ex-7M/Ex-11M (exon 6 and 10 in anterior pituitary). Data are representative of two independent experiments;

FIG. 17 is the nucleic acid sequences encoding a 400 amino acids protein (A) or encoding a 416 amino acids protein (B) of human renal medulla GHRH receptor;

FIG. 18 is the deduced protein sequences of human renal medulla GHRH receptor of 400 amino acids (A) or 416 amino acids (B); and

FIG. 19 illustrates a comparison of human GHRH-R protein sequence of 400 amino acids (A) or 416 amino acids (B) between anterior pituitary and the renal medulla.

### DETAILED DESCRIPTION

In order to provide a clear and consistent understanding of the terms used in the present disclosure, a number of definitions are provided below. Moreover, unless defined otherwise, all technical and scientific terms as used herein have the same meaning as commonly understood to one of ordinary skill in the art to which this invention pertains.

In one aspect thereof, the present invention relates to a method for protecting (and/or treating) a mammal against oxidative renal damage. The method may comprise the step of administering an effective amount of a ligand to GHRH renal receptor to the mammal.

Oxidative stress occurs inside cells or tissues when production of oxygen radicals exceeds their antioxidant capacity. Excess of free radicals may damage essential macromolecules such as, for example, protein, lipids and DNA, leading to abnormal gene expression, disturbance in receptor activity and signaling, apoptosis, immunity perturbation, mutagenesis, and protein or lipofushin deposition. Numerous human diseases involve localized or general oxidative stress. In many serious diseases such as cancer, ocular degeneration (age-related macular degeneration or cataract) and neurodegenerative diseases (ataxia, amyotrophic lateral sclerosis, Alzheimer's disease), oxidative stress is one of the primary disease factor. In various other diseases, oxidative stress occurs secondary to the initial disease and plays an important in role in immune and vascular complications, such as, for example, in AIDS, septic shock, Parkinson's disease, diabetes and renal failure. It is also the case in aging, were accumulation of cellular oxidative stress is considered as a key element in the deterioration of tissues, organs and systems. In an embodiment of the present invention, the oxidative stress may be renal oxidative stress and/or renal oxidative damage. Renal oxidative stress and/or renal oxidative damage may lead to impairment and/or loss of renal function. Renal function is an indication of the state of the kidney and its role in renal physiology. For example, glomerular filtration rate (GFR) may be used to describe the flow rate of filtered fluid through the kidney and assess renal function. Creatinine clearance rate (CCr) is also a marker for renal function and corresponds to the volume of blood plasma that is cleared of creatinine per unit time and is a useful measure for approximating the GFR. Both GFR and CCr may be accurately calculated by comparative measurements of substances in the blood and urine. An exemplary oxidative damage in kidneys may be due to exaggerated renal medullary osmolality.

In an embodiment of the present invention, oxidative renal damage may affect Henle's loop cells. In another embodiment, the Henle's loop cells affected by oxidative renal damage may more specifically be thin limb Henle's loop cells. In yet a further embodiment, the thin limb Henle's loop cells affected by oxidative renal damage may be ascending thin limb Henle's loop cells.

According to the present invention, a mammal in need may be identified by various means and methods prior to administration of a GHRH-R ligand, for example, by determining kidney function. The mammal may also be identified by determining the presence of markers associated with oxidative stress (damage) to kidney cells and/or kidney function. In an exemplary embodiment of the present invention, the mammal may be a human being.

According to the present invention, the term “marker” means any marker of kidney function and/or any stress marker known in the art or as described herein. Stress markers may be oxidatively damaged proteins and/or lipids, active oxygen species (hydroxy radicals, alkoxy radicals, hydroperoxy radicals, peroxy radicals, iron-oxygen complexes, superoxides, hydrogen peroxide, hydroperoxides, singlet oxygen and ozone) or free radicals (lipid radicals and the like). For example, concentrations of two major aldehydic lipid peroxidation (LPO) products, 4-hydroxynonenal (HNE) and malondialdehyde (MDA), and of protein carbonyls may be analyzed as parameters of oxidative stress related to kidney function. Kidney function markers include for example, creatinin, urea, apolipoprotein A-IV. Measurements of these markers (serum measurement, urinary measurement, etc) may be useful to identify a mammal in need (patients) for which the present invention is desirable.

These markers may be measured in vivo or in bodily fluids such as in urine, serum and/or plasma. Indicators of deterioration of kidney cells such as Henle's loop ascending thin limb cells also include change in urine osmolarity, volume/time urine production and content of urine. As such specific markers alone and/or in combination with indicators of general kidney function may be used to identify the population of patients for which treatment is sought or desirable. Several markers of kidney function are known in the art and markers of oxidative stress (damage to DNA, lipids and/or proteins) to kidney cells have been identified. As such, urinary measurements of these markers may be useful to identify patients for which the present invention is desirable.

In an exemplary embodiment, the total antioxidant status (TAS) of the mammal may be measured. This assay is based on the capacity of a plasma sample obtained from the mammal to inhibit the formation of 2,2′-azinobis (3-ethylbenzothiazoline-6-sulfonate) (ABTS) radicals in the presence of H₂O₂ and metmyoglobine⁶⁰. The percentage of inhibition corresponds to the TAS value expressed in Trolox equivalent. Upon determining the TAS, other plasmatic components are taken into account, namely; concentration of plasma albumin and uric acid. The TAS will thus be determined by the following formula:

TAS=TAS measured−[(Albumin mmol/l×0.69)+Uric acid mmol/l×1)]

The oxidative capacity of albumin is 0.69 mmol/L Trolox equivalent while the oxidative capacity of uric acid is 1 mmol/L Trolox equivalent. Plasma uric acid levels may be measured by HPLC.

Oxidative stress to lipids may be determined by evaluating the amount of F2-isoprostane (isomers of prostaglandin F2 (PG F2)) which are formed by the non-enzymatic oxidation of arachnidoic acid under condition of oxidative stress. More particularly, 8-iso-PGF2, the most abundant member of this family is a reliable marker of in vivo oxidative stress to plasma and cellular lipids. To that effect, 8-iso-PGF2 may be extracted from the organic phase of an esterified urine sample (with ester pentafluorbenzyl) and analyzed by gaz chromatography coupled to mass spectroscopy (GC/MS) as per Nourooz-Zadeh et al.

Oxidative stress to DNA may be determined by measuring the presence of 8-oxo-dGuo in urine. The presence of this product may be detected by HPLC with electrochemical detection as per Arthur et al and Reznick et al.^{(65, 66)}

A mammal of the invention may suffer or may be susceptible of suffering from a disease that may consist in aging- and frailty-related nephropathy and renal failure, diabetes insipidus, diabetes type I, diabetes II, renal disease glomerulonephritis, bacterial or viral glomerulonephritides, IgA nephropathy, Henoch-Schonlein Purpura, membranoproliferative glomerulonephritis, membranous nephropathy, Sjogren's syndrome, nephrotic syndrome minimal change disease, focal glomerulosclerosis and related disorders, acute renal failure, acute tubulointerstitial nephritis, pyelonephritis, genitourinary (GU) tract inflammatory disease, pre-clampsia, renal graft rejection, leprosy, reflux nephropathy, nephrolithiasis, genetic renal disease, medullary cystic, medullar sponge, polycystic kidney disease, autosomal dominant polycystic kidney disease, autosomal recessive polycystic kidney disease, tuberous sclerosis, von Hippel-Lindau disease, familial thin-glomerular basement membrane disease, collagen III glomerulopathy, fibronectin glomerulopathy, Alport's syndrome, Fabry's disease, Nail-Patella Syndrome, congenital urologic anomalies, monoclonal gammopathies, multiple myeloma, amyloidosis and related disorders, febrile illness, familial Mediterranean fever, HIV infection, AIDS, inflammatory disease, systemic vasculitides, polyarteritis nodosa, Wegener's granulomatosis, polyarteritis, necrotizing and crescentic glomerulonephritis, polymyositis-dermatomyositis, pancreatitis, rheumatoid arthritis, systemic lupus erythematosus, gout, blood disorders, sickle cell disease, thrombotic thrombocytopenia purpura, hemolytic-uremic syndrome, acute cortical necrosis, renal thromboembolism, trauma and surgery, extensive injury, burns, abdominal and vascular surgery, induction of anesthesia, side effect of drug abuse or use of including those generating renal oxidative stress and toxicity such as antibiotics and cancer chemotherapeutic agents, malignant disease, adenocarcinoma, melanoma, lymphoreticular, multiple myeloma, circulatory disease, myocardial infarction, cardiac failure, peripheral vascular disease, hypertension, coronary heart disease, non-atherosclerotic cardiovascular disease, atherosclerotic cardiovascular disease, skin disease, psoriasis, systemic sclerosis, respiratory disease, chronic obstructive pulmonary disease, obstructive sleep apnea, hypoxia at high altitude or endocrine disease, acromegaly, diabetes mellitus and/or conditions related to antibiotic toxicity, infection, inflammation, ischemia. A mammal of the invention may also be a mammal subjected to chronic hemodialysis.

By “protecting” a mammal against oxidative renal damage, it is meant a process by which oxidative stress may partially or totally be prevented from damaging renal cells in a mammal. For example, by administering an effective amount of a ligand to GHRH renal receptor in a mammal, renal oxidative damage may partially or totally be prevented. By “treating” a mammal against oxidative renal damage it is meant a process by which the oxidative renal damage is reduced either partially or totally. Treating oxidative renal damage also encompasses a process by which the symptoms of oxidative renal damage in a mammal may not worsen, may remain stable, may be reduced and/or may be completely eliminated.

As used herein, a “ligand” to the renal GHRH receptor may be native GHRH (SEQ ID NO.:1), a biologically active fragment of GHRH and/or a GHRH agonist thereof. Exemplary embodiments of a GHRH biologically active fragment may include for example, SEQ ID NO.:2 and/or 3. Exemplary embodiment of GHRH agonist may include for example, any one of SEQ ID NO.:4 to 9. Specific embodiments of GHRH agonists may include for example, any one of SEQ ID NO.:4 to 6 wherein Xaa is absent. GHRH agonist of the invention may be capable of activating and/or upregulating renal GHRH receptor.

In a further exemplary embodiment of the invention, the ligand may be SEQ ID NO.:10, wherein Xaa2 is D-Ala and wherein the remaining amino acid sequence is identical to SEQ ID NO.:1 or 3.

In an additional exemplary embodiment of the invention, the ligand may be SEQ ID NO.: 10, wherein Xaa10 is D-Tyr and wherein the remaining amino acid sequence is identical to SEQ ID NO.:1 or 3.

In another exemplary embodiment of the invention, the ligand may be SEQ ID NO.: 10, wherein Xaa15 is D-Ala and wherein the remaining amino acid sequence is identical to SEQ ID NO.:1 or 3.

In another exemplary embodiment of the invention, the ligand may be SEQ ID NO.:10, wherein Xaa22 is Lys and wherein the remaining amino acid sequence is identical to SEQ ID NO.:1 or 3.

In yet another exemplary embodiment of the invention, the ligand may be SEQ ID NO.:10, wherein Xaa2 is D-Ala and/or Xaa10 and/or D-Tyr and/or Xaa15 is D-Ala and/or Xaa22 is Lys and wherein the remaining amino acid sequence is identical to SEQ ID NO.:1 or 3.

In an additional embodiment, the ligand may be SEQ ID NO.:10, wherein Xaa8 is Ala and/or Xaa9 is Ala, and/or Xaa15 is Ala and/or Xaa22 is Ala.

In yet an additional embodiment, the ligand may be SEQ ID NO.:10, wherein Xaa22 is Lys.

As used herein, an “effective amount” is the necessary quantity to obtain positive results without causing excessively negative effects in the host to which a ligand to GHRH renal receptor may be administered. An exemplary effective amount encompassed in the present invention may relate to a quantity which may be sufficient to protect and/or treat a mammal against oxidative renal damage, prevent the death of kidney cells due to oxidative stress and/or promote regeneration of kidney cells.

An effective amount may be administered in one or more administrations, according to a regimen. The privileged method of administration and the quantity that may be administered is function of many factors. Among the factors that may influence this choice are, for example, the condition, the age and the weight of the host to which a ligand of renal GHRH receptor is to be administered. Various routes of administration may include, for example, parenteral, pulmonary, nasal, oral, transmucosal, transdermal, intramuscular, intravenous, intradermal, subcutaneous and/or intraperitonal administration.

In an embodiment of the present invention, an effective amount may not be substantially active against anterior pituitary GHRH receptor. An effective amount may have, for example, a protective effect substantially similar to a subcutaneous 1.0 mg rat GHRH(1-29)NH₂ dose per kilogram of body weight per day or lower, in a Sprague Dawley rat submitted to a high-salt diet. An effective amount may preferentially have, for example, a protective effect substantially similar to a subcutaneous 0.5 mg rat GHRH(1-29)NH₂ dose per kilogram of body weight per day or lower, in a Sprague Dawley rat submitted to a high-salt diet.

In another aspect, the present invention relates to a method for preventing (lowering) the death of kidney cells (and/or loss of kidney cell function) due to oxidative stress in a mammal in need thereof. The method may comprise administering to the mammal a ligand to GHRH renal receptor.

In a further aspect, the present invention relates to a method of promoting regeneration of kidney cells and/or function in a mammal in need thereof. The method may comprise administering a ligand to the GHRH renal receptor the mammal.

In yet a further aspect thereof, the present invention relates to the new isolated polypeptidic sequence of rat GHRH receptor, antibody that may bind same, nucleic acids that may encode same and/or vectors that may comprise the nucleic acids of the present invention.

“Polypeptides” refers to any peptide or protein comprising two or more amino acids joined to each other by peptide bonds or modified peptide bonds (i.e., peptide isosteres). “Polypeptide” refers to both short chains, commonly referred as peptides, oligopeptides or oligomers, and to longer chains generally referred to as proteins.

The present invention therefore relates in a further aspect, to an isolated polypeptide that may comprise SEQ ID NO.: 13, SEQ ID NO.:16 or SEQ ID NO.:17, a SEQ ID NO.13, SEQ ID NO.:16 or SEQ ID NO.:17 fragment and/or a SEQ ID NO.:13, SEQ ID NO.:16 or SEQ ID NO.:17 analog. The polypeptide of the invention may be free of a N-terminal amino acid sequence of a pituitary GHRH receptor. Exemplary embodiments of the N-terminal amino acid sequence found in pituitary GHRH-R and not in renal GHRH-R are provided in SEQ ID NO.:14 and 15 for rat GHRH-R and SEQ ID NO.: 22 and SEQ ID NO.: 23 for human GHRH-R. It is to be understood that as used herein “pituitary GHRH-R” is meant to encompass both rat and/or human pituitary GHRH-R. When a polypeptide of the invention is a rat polypeptide (for example SEQ ID NO.:13) it may be free of a N-terminal amino acid sequence of a pituitary GHRH receptor from a rat. When a polypeptide of the invention is a human polypeptide (for example, SEQ ID NO.:16 or SEQ ID NO.:17) it may be free of a N-terminal amino acid sequence of a pituitary GHRH receptor from a human.

Polypeptides encompassed by the present invention may thus comprise and/or consist in SEQ ID NO.:13, SEQ ID NO.:16 or SEQ ID NO.:17, analogues of SEQ ID NO.:13, SEQ ID NO.:16 or SEQ ID NO.:17 and/or fragments thereof. Polypeptides may also comprise additional amino acids at the amino or carboxy end. These additional amino acids may be different than those of pituitary GHRH-R.

As used herein, an “analogue” is to be understood as a polypeptide which is substantially identical to an original sequence. An analogue may comprise one or more modification in the amino acid sequence in comparison with the original sequence, for example, amino acid addition(s), deletion(s), insertion(s), conservative or non-conservative substitution(s), one or more modification in the backbone or side-chain of one or more amino acid, or an addition of a group or another molecule to one or more amino acids (side-chains or backbone). An “analogue” is therefore understood herein as a molecule having a biological activity similar to that of a polypeptide described herein. Exemplary embodiments of the polypeptide analogs of SEQ ID NO.13, SEQ ID NO.:16 or SEQ ID NO.:17 may be those which possess between 60% to 100% but preferably at least 70% amino acid identity with SEQ ID NO.13, SEQ ID NO.:16 or SEQ ID NO.:17, at least 75% amino acid identity with SEQ ID NO.13, SEQ ID NO.:16 or SEQ ID NO.:17, at least 80% amino acid identity with SEQ ID NO.13, SEQ ID NO.:16 or SEQ ID NO.:17, at least 85% amino acid identity with SEQ ID NO.13, SEQ ID NO.:16 or SEQ ID NO.:17, at least 90% amino acid identity with SEQ ID NO.13, SEQ ID NO.:16 or SEQ ID NO.:17 and/or at least 95% amino acid identity with SEQ ID NO.13, SEQ ID NO.:16 or SEQ ID NO.:17. The present invention relates to and explicitly incorporates each and every specific member and combination of sub-ranges whatsoever. Thus, any specified range is to be understood as a shorthand way of referring to each and every member of a range or group individually as well as each and every possible sub-ranges encompassed therein; and similarly with respect to any sub-ranges therein. As such, with respect to amino acid identity, at least 70% encompasses any value between 70% to 100%, for example, 71%, 71.5%, 72%, 73%, 77%, 83%, etc.

An “analogue” may have sequence similarity and/or sequence identity with that of an original sequence or a portion of an original sequence. The degree of similarity between two sequences is based upon the percentage of identities (identical amino acids) and of conservative substitution. Similarity or identity may be compared, for example, over a region of 10, 20, 100 amino acids or more (and any number therebetween) or over the total length of the protein. Identity may include amino acids which are identical to the original peptide and which may occupy the same or similar position when compared to the original polypeptide. For example, a polypeptide may share 60% sequence identity with another and may have one or more conservative amino acids substitutions in the non-identical positions which may result in the polypeptide having at least 60.1%, 65%, 70%, 80%, 85%, 90% etc., sequence similarity. The remaining amino acids of the polypeptide which are neither identical nor similar may be occupied by non-conservative amino acid substitutions or alternatively by gaps (no amino acids).

Percent identity may be determined, for example, with n algorithm GAP, BESTFIT, or FASTA in the Wisconsin Genetics Software Package Release 7.0, using default gap weights. Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs can be found at the following internet address: http://www.ncbi.nlm.qov/cgi-bin/BLAST.

As is generally understood, naturally occurring amino acids may be sub-classified as acidic, basic, neutral and polar, or neutral and non-polar. Furthermore, three of the encoded amino acids are aromatic. It may be of use that encoded polypeptides differing from the determined polypeptide of the present invention contain substituted codons for amino acids, which are from the same type or group as that of the amino acid to be replaced. Thus, in some cases, the basic amino acids Lys, Arg and His may be interchangeable; the acidic amino acids Asp and Glu may be interchangeable; the neutral polar amino acids Ser, Thr, Cys, Gln, and Asn may be interchangeable; the non-polar aliphatic amino acids Gly, Ala, Val, Ile, and Leu are interchangeable but because of size Gly and Ala are more closely related and Val, Ile and Leu are more closely related to each other, and the aromatic amino acids Phe, Trp and Tyr may be interchangeable.

It is known in the art that analogues may be generated by substitutional mutagenesis and retain the biological activity of the polypeptides of the present invention. These analogues have at least one amino acid residue in the protein molecule removed and a different residue inserted in its place. Examples of substitutions identified as “conservative substitutions” are shown in Table 1. If such substitutions result in a change not desired, then other type of substitutions, denominated “exemplary substitutions” in Table 1, or as further described herein in reference to amino acid classes, are introduced and the products screened.

TABLE 1
Amino Acid Substitution

| Original | Exemplary | Conservative |
|---|---|---|
| residue | substitution | substitution |
| Ala (A) | Val, Leu, Ile | Val |
| Arg (R) | Lys, Gln, Asn | Lys |
| Asn (N) | Gln, His, Lys, Arg | Gln |
| Asp (D) | Glu | Glu |
| Cys (C) | Ser | Ser |
| Gln (Q) | Asn | Asn |
| Glu (E) | Asp | Asp |
| Gly (G) | Pro | Pro |
| His (H) | Asn, Gln, Lys, Arg | Arg |
| Ile (I) | Leu, Val, Met, Ala, Phe, | Leu |
| | norleucine | |
| Leu (L) | Norleucine, Ile, Val, Met, | Ile |
| | Ala, Phe | |
| Lys (K) | Arg, Gln, Asn | Arg |
| Met (M) | Leu, Phe, Ile | Leu |
| Phe (F) | Leu, Val, Ile, Ala | Leu |
| Pro (P) | Gly | Gly |
| Ser (S) | Thr | Thr |
| Thr (T) | Ser | Ser |
| Trp (W) | Tyr | Tyr |
| Tyr (Y) | Trp, Phe, Thr, Ser | Phe |
| Val (V) | Ile, Leu, Met, Phe, Ala, | Leu |
| | norleucine | |

A “fragment” is to be understood herein as a polypeptide originating from a portion of an original or parent sequence or from an analogue of said parent sequence. Fragments encompass polypeptides having truncations of one or more amino acids, wherein the truncation may originate from the amino terminus (N-terminus), carboxy terminus (C-terminus), or from the interior of the protein. A fragment may comprise the same sequence as the corresponding portion of the original sequence. Fragments may be useful for example, in the generation of antibodies and/or for testing antibodies.

In another aspect, the present invention relates to an isolated nucleic acid sequence encoding a polypeptide described herein (e.g., SEQ ID NO.: 13, a SEQ ID NO:13 fragment and/or a SEQ ID NO.:13 analog, SEQ ID NO.: 16, a SEQ ID NO.16 fragment and/or a SEQ ID NO.:16 analog, SEQ ID NO.: 17, a SEQ ID NO.17 fragment and/or a SEQ ID NO.:17 analog, etc.).

In yet another aspect, the present invention relates to an isolated nucleic acid that may be selected from the group that may consist of a. a polynucleotide comprising or consisting of SEQ ID NO.11, SEQ ID NO.12, SEQ ID NO.:18, SEQ ID NO.:19, SEQ ID NO.:20 or SEQ ID NO.:21, b. a polynucleotide comprising a sequence substantially identical to SEQ ID NO.11, SEQ ID NO.12, SEQ ID NO.:18, SEQ ID NO.:19, SEQ ID NO.:20 or SEQ ID NO.:21, and/or c. a polynucleotide comprising a sequence substantially complementary to a. or b., and/or d. a fragment of any one of a., b. or c.

As used herein the term “polynucleotide” or “nucleic acid” generally refers to any polyribonucleotide or polydeoxyribonucleotide, which may be unmodified RNA or DNA, or modified RNA or DNA. “Polynucleotides” or “nucleic acids” include, without limitation single- and double-stranded DNA, DNA that is a mixture of single- and double-stranded regions, single- and double-stranded RNA, and RNA that is a mixture of single- and double-stranded regions, hybrid molecules comprising DNA and RNA that may be single-stranded or, more typically, double-stranded or a mixture of single- and double-stranded regions. In addition, “polynucleotide” or “nucleic acid” refers to triple-stranded regions comprising RNA or DNA or both RNA and DNA. The term “polynucleotide” or “nucleic acid” also includes DNAs or RNAs containing one or more modified bases and DNAs or RNAs with backbones modified for stability or for other reasons. “Modified” bases include, for example, tritylated bases and unusual bases such as inosine. A variety of modifications may be made to DNA and RNA; thus “polynucleotide” embraces chemically, enzymatically or metabolically modified forms of polynucleotides as typically found or not in nature, as well as the chemical forms of DNA and RNA characteristic of viruses and cells. “Polynucleotide” or “nucleic acid” includes but is not limited to linear and end-closed molecules. “Polynucleotide” or “nucleic acid” also embraces relatively short polynucleotides, often referred to as oligonucleotides.

In accordance with the present invention, the nucleic acid may be free of a nucleic acid sequence encoding a N-terminal amino acid sequence of a pituitary GHRH receptor as described herein.

In an additional aspect, the present invention relates to a vector that may comprise the nucleic acid sequences described herein. “Vector” refers to an autonomously replicating DNA and/or RNA molecule into which foreign DNA and/or RNA fragments are inserted and then propagated in a host cell for either expression or amplification of the foreign DNA and/or RNA molecule. The term vector may comprise, for example and without limitation, a plasmid (e.g., linearized or not) that may be used to transfer DNA sequences from one organism to another.

The term “substantially identical” used to define the polynucleotides of the present invention refers to polynucleotides which have, for example, from 50% to 100% sequence identity and any range therebetween but preferably at least 80%, at least 85%, at least 90%, at least 95% sequence identity and also include 100% identity with that of an original sequence (including sequences 100% identical over a portion of the sequence or over the entire length of the polynucleotide sequence). The present invention relates to and explicitly incorporates each and every specific member and combination of sub-ranges whatsoever. Thus, any specified range is to be understood as a shorthand way of referring to each and every member of a range or group individually as well as each and every possible sub-ranges encompassed therein; and similarly with respect to any sub-ranges therein. As such, with respect to polynucleotides sequence identity, at least 80% encompasses any value between 80% to 100%, for example, 81%, 81.5%, 82%, 83%, 87%, 93%, etc.

“Substantially identical” and “substantially complementary” polynucleotide

(nucleic acid) sequences may be identified by providing a probe of about 10 to about 20 or about 10 to about 25 nucleotides long (or longer) based on the sequence of SEQ ID NO:11 or 12 or complementary sequence thereof and hybridizing a library of polynucleotide (e.g., cDNA or else) originating from another species, tissue, cell, individual, etc. A polynucleotide which hybridizes under highly stringent conditions (e.g., 6XSCC, 65° C.) to the probe may be isolated and identified using methods known in the art.

As used herein the terms “sequence complementarity” refers to (consecutive) nucleotides of a nucleotide sequence which are complementary to a reference (original) nucleotide sequence. The complementarity may be compared over a region and/or over the total length of a nucleic acid sequence.

The term “substantially complementary” used to define the polynucleotides of the present invention refers to polynucleotides which have, for example, from 50% to 100% sequence complementarity and any range therebetween but preferably at least 80%, at least 85%, at least 90%, at least 95% sequence complementarity and also include 100% complementarity with that of an original sequence (including sequences 100% complementarity over the entire length of the polynucleotide sequence).

A further aspect of the present invention relates to an antibody capable of specific binding to polypeptides described herein. For example, an antibody capable of specific binding to SEQ ID NO.: 13, SEQ ID NO.:16 and/or SEQ ID NO.:17. Exemplary embodiments of such antibodies are those which do not substantially bind to a GHRH pituitary receptor but bind to a GHRH renal receptor.

As used herein the term “antibody” encompasses a monoclonal antibody, a polyclonal antibody, a single chain antibody, a chimeric antibody, a humanized antibody, a deimmunized antibody, an antigen-binding fragment, a Fab fragment, a F(ab′)₂ fragment, a Fv fragment, complementarity determining regions (CDRs), or a single-chain antibody comprising an antigen-binding fragment (e.g., a single chain Fv).

The antibody may originate for example, from a mouse, rat or any other mammal or from other sources such as through recombinant DNA technologies. The antibody may also be a human antibody which may be obtained, for example, from a transgenic non-human mammal capable of expressing human Ig genes. The antibody may also be a humanised antibody which may comprise, for example, one or more complementarity determining regions of non-human origin. It may also comprise a surface residue of a human antibody and/or framework regions of a human antibody. The antibody may also be a chimeric antibody which may comprise, for example, variable domains of a non-human antibody and constant domains of a human antibody.

The minimum size of peptides useful for obtaining antigen specific antibodies may vary widely. The minimum size must be sufficient to provide an antigenic epitope that is specific to the protein and/or polypeptide. The maximum size is not critical unless it is desired to obtain antibodies to one particular epitope. For example, a large polypeptide may comprise multiple epitopes, one epitope being particularly useful and a second epitope being immunodominant, etc. Typically, antigenic peptides selected from the present proteins and polypeptides will range without limitation, from 5 to about 100 amino acids in length or may comprise the whole protein. More typically, however, such an antigenic peptide will be a maximum of about 50 amino acids in length, and preferably a maximum of about 30 amino acids. It is usually desirable to select a sequence of about 6, 8, 10, 12 or 15 amino acids, up to about 20 or 25 amino acids (and any number therebetween).

To obtain polyclonal antibodies, a selected animal may be immunized with a protein and/or polypeptide. Serum from the animal may be collected and treated according to known procedures. Polyclonal antibodies to the protein or polypeptide of interest may then be purified by affinity chromatography. Techniques for producing polyclonal antisera are well known in the art.

Monoclonal antibodies (MAbs) may be made by one of several procedures available to one of skill in the art, for example, by fusing antibody producing cells with immortalized cells and thereby making a hybridoma. The general methodology for fusion of antibody producing B cells to an immortal cell line is well within the province of one skilled in the art. Another example is the generation of MAbs from mRNA extracted from bone marrow and spleen cells of immunized animals using combinatorial antibody library technology.

In yet a further aspect, the present invention relates to an isolated cell expressing the polynucleotides and/or polypeptides of the invention. In an exemplary embodiment, the isolated cell may be a renal cell.

In another aspect, the present invention relates to an assay for identifying a ligand capable of specific binding to polypeptides of the present invention and not to pituitary GHRH receptor. This assay may comprise contacting a test ligand (e.g. a library of test ligands) with at least one polypeptide of the present invention, measuring binding of the test ligand to the polypeptide and/or determining the identity of the test ligand. The assay may further comprise a step of selecting a ligand which does not substantially bind to pituitary GHRH-R.

In a further aspect, the present invention relates to a method for the diagnosis of renal oxidative stress and/or damage. The method may comprise the step of detecting (measuring) the expression of renal GHRH receptor by any means known to a person skilled in the art, wherein downregulation of GHRH-R in early stages of suspected renal oxidative stress/damage and/or upregulation of GHRH-R in late stages of suspected renal oxidative stress/damage is indicative of renal oxidative stress and/or damage. For example, the expression of GHRH-R may be detected at the RNA (for example, mRNA) and/or protein level. The expression of mRNAs may be detected using methods which are known in the art, such as, for example and without limitation, hybridization analysis using oligonucleotide probes, reverse transcription and in vitro nucleic acid amplification methods. The expression of proteins of the invention may be detected, for example and without limitation, using antibodies specific for renal GHRH-R in a variety of methods known to a person skilled in the art of measuring polypeptides, including immunoblotting, ELISA, radioimmunassay, and FACS, etc. The present invention also encompasses a diagnosis kit that may comprise any detection reagents used to detect the expression of renal GHRH receptor (for example, a renal GHRH-R specific probe and/or a renal GHRH-R specific antibody) as well as other suitably packaged reagents and materials needed for detection of renal GHRH receptor expression.

### EXAMPLES

The following examples are presented to illustrate the invention but it is not to be considered as limited thereto.

### Example 1

### Materials and Methods

All material and methods used in subsequent examples are herein described for reference.

Animal Handling, Treatments and Tissue Preparations

Two-month-old male Sprague Dawley rats (Charles River Canada, St-Constant, QC) were kept in temperature- (22° C.), humidity- (65%) and lighting- (12h cycles; lights on at 0700 h) controlled rooms and had free access to standard rat chow (2018 Tecklad global 18% protein rodent diet, containing 0.23% Na⁺ and 0.4% Cl⁻; Harlan Tecklad, Madison, Wis.) and tap water. Rats were acclimatized ≈3 days before going on a high-NaCl diet or water deprivation. Rats fed the custom-made high-NaCl diet for 2, 7 or 14 (8% NaCl; Harlan Tecklad) were compared to rats fed the custom-made control diet (0.3% NaCl; Harlan Tecklad). They had free access to water. Rats deprived of water for 3 or 5 days had free access to 2018 Tecklad rat chow. Rats used in the first series of experiments, to quantify GHRH-R mRNA levels following a 8%-NaCl diet or a water deprivation, were housed individually in metabolic cages for the entire duration of intervention. Body weight (BW), food and water intakes, and urine volume were recorded daily, and Na⁺ levels were analyzed on the last 24h urine sample before sacrifice. Rats used in the 8%-NaCl diet/GHRH study were housed individually in plastic cages and BW and food intake were recorded daily. Rats were sacrificed in a block-design fashion between 0900-1130 h, by rapid decapitation. Pituitaries, kidneys and livers were excised immediately and anterior pituitaries and renal medullas dissected out. Tissues were snap-frozen in liquid nitrogen and stored at −80° C. until RNA extraction. For isolation of thin and thick limb cells, renal medullas were dissected out rapidly, washed and minced in ice-cold oxygenated HEPES-Ringer buffer (290 mosm, pH 7.4). For isolation of thick ascending limb cells, inner stripes of outer medullas were dissected out and kept in ice-cold oxygenated Hanks solution.⁵⁵ For in vivo BrdU-labeling experiments, rats were fed a 0.3%- or 8%-NaCl chow for 2 days (day 1, day 2) and received in the back a subcutaneous (sc) injection of 1.0 mg rGHRH(1-29)NH₂/kg BW, solubilized in normal physiological saline (GHRH-treated) or an isovolumetric amount of saline (control). rGHRH-(1-29)NH₂ (synthesized in our laboratory)⁵⁶ was solubilized each morning just before treatment and kept on ice. Rats were injected intra-peritoneally on the morning of day 3, with 100 mg 5-bromo2′-deoxy-uridine/kg BW (30 mg ultrapure BrdU/1 ml in normal saline; Sigma-Aldrich Canada Ltd, Oakville, ON), 2 h prior to sacrifice. For in vivo GHRH treatment, rats received in the back a subcutaneous (sc) injection of 1.0 mg rGHRH(1-29)NH₂/kg BW daily.

Porcine anterior pituitaries and renal medullas from Yorkshire-Landrace pigs (≈107 kg, ≈150-day-old) were dissected out at a local slaughter house, snap-frozen in liquid nitrogen and stored at −80° C. until RNA extraction.

Isolation of Thin Limbs of Henle's Loop Cells

Cell dispersion of minced medullas to obtain semi-purified thin limb cells was performed as previously described.³³ These cells were used immediately for in vitro determination of basal and rGHRH(1-29)NH₂-induced cAMP levels or purified by differential centrifugation for immunocytochemistry, using a continuous gradient of Nycodenz. The gradient was prepared as described by Grupp et al.⁵⁵ and thin limb cells were recovered in fraction I of the gradient after centrifugation at 1500 g (16° C., 45 min) and washed twice in HEPES-Ringer buffer (430 g, 16° C., 10 min). Cell viability, assessed by the Trypan Blue exclusion method, was around 95%. When purified thin limb cells were cultured, isolation and purification steps were performed under sterile conditions and media containing antibiotics.

Isolation of Thick Limbs of Henle's Loop Cells

The inner stripe of outer medullas were dissected out using an optical stereomicroscope, minced and kept in oxygenated Hanks solution. Short time cell dispersion was performed as previously described,⁵⁷ and dispersed cells were poured on the top of a 100 μm-pore nylon membrane (Millipore, Nepean, ON, CA) and washed with Hanks-1% BSA (Sigma-Aldrich) solution using a syringe adapted to a 25G needle. Thick ascending HL cells were detached from the membrane by washing with Hanks-1% BSA solution. The suspension was centrifuged at 80 g for 5 min (4° C.) and the pellet resuspended in ice-cold Hanks solution. Cell viability was determined as above and was similar.

Immunocytochemical Procedures

Specific markers of descending (anti-aquaporin-1 antiboby (Ab)) and ascending thin limb (anti-CIK-K1/-K2 (CIC-K) Ab) cells (Alamone Labs, Jerusalem, Israel) were directly conjugated to the fluorochrome Alexa 488, using the Alexa™ 488 Protein Labeling Kit (Molecular Probes, Eugene, Oreg.), according to the manufacturer's protocol. Labeled antibodies were purified on molecular size exclusion spin columns, supplied with the kit (1100 g, 5 min). Purified thin limb cells were fixed in fresh 4% paraformaldehyde-phosphate-buffered saline (20 min, RT), washed twice with PBS and centrifuged (800 g, 4° C., 5 min). Thin limb cells (≈2200,000) were spun onto glass slides by cytocentrifugation (32 g, RT, 2 min) and permeabilized in 0.2% Triton X-100 (Sigma-Aldrich) for 15 min (RT). Slides were washed in PBS (4×5 min, RT), blocked with 5% (wt/vol.) BSA-PBS (30 min, RT) and washed in PBS (2×10 min). GHRH-R was detected using 0.5 μg of the purified anti-GHRH-R(392-404) polyclonal antibody³³ in 100 μl PBS, containing 1% BSA, incubated overnight (ON) at 4° C., in a humid atmosphere (7, 8). Cells were rinsed in PBS (2×10 min, RT), incubated 60 min (RT), in the presence of Alexa 568™-conjugated goat anti-rabbit IgGs (Molecular Probes) (1:15000 in PBS-BSA 1% buffer) and washed in PBS (2×10 min). Descending thin limb cells were then visualized using a rabbit polyclonal Alexa 488™-conjugated anti-aquaporin-1 antibody (1:2000 diluted in PBS-BSA 1%, 60 min, 37° C.) while ascending thin limb cells were visualized using a rabbit polyclonal Alexa 488™-conjugated anti-CIC-K antibody (1:500 diluted in PBS-BSA 1%, 60 min, 37° C.). A final wash of slide-mounted cells was done in PBS (2×10 min). Specificity of labeling was assessed by substituting GHRH-R (392-404) polyclonal antibodies with normal IgGs. Another series of experiments, using a similar procedure as described above, was performed to determine whether or not immunoreactive GHRH was present in ascending thin limb cells. An anti-rat GHRH(1-43)OH antibody (0.5 μg/100 μl; Bachem Biosciences Inc, King of Prussia, Pa.) and a secondary Alexa 568-conjugated goat-anti-rabbit IgGs (1/7500, Molecular Probes) were used. One μM of 4,6-diamodino-2-phenylindole dihydrochloride (DAPI, Molecular Probes) was added for the last 30 min of incubation to stain nuclei. All procedures with fluorescent probes were performed in the dark. Cells were visualized using a Nikon Eclipse E600 (Nikon Canada Inc., Montreal, QC) fluorescence/light microscope equipped with filters for excitation/emission of fluorescein (485/520 nm) and Texas Red (595/660) and DAPI (360/460 nm).

BrdU immunolabeling (Roche Diagnostics, Laval, QC, CA) was used to quantify DNA repair/synthesis in purified thin limb cells from rat submitted 2 days to a 8%- or 0.3%-NaCl diet and injected with GHRH or saline. The cells were purified as above, cultured 16h in DMEM/F12, containing 25 mM glucose, 10% fetal bovine serum, 1% penicillin-streptomycin, 0.1% amphotericin,³³ on coverslip in 24-wells sterile culture plates (≈1×10⁶ cells/well). They were fixed with fresh 4% paraformaldehyde (500 μl/well, 15 min, RT) and washed using the washing buffer supplied with the kit (2×5 min, 500 μl/well). They were subsequently incubated in blocking buffer as above (30 min, RT, 500 μl/well). Immunolabelling was performed with the primary antibody anti-BrdU (dilution 1:10 in the incubation buffer, 30 min, 37° C., 150 μl/well). Non-specific fluorescence was determined by substituting the primary antibody by normal rabbit IgGs. Immunodetection was performed after washing by adding a secondary anti-rabbit-IgG antibody coupled to fluorescein (30 min, 37° C., 150 μl/well). All steps using fluorescent labeling was performed in the dark. Nuclei and mitochondria were labeled using 1 μM DAPI and 10 nM of Mitotracker red CMXRos (Molecular Probes, Oreg., USA; PBS 1×, 15 min, RT, 200 μl/well). After final washing, cells on coverslips were dried and mounted with Prolong mounting medium/Prolong antifade (Molecular Probes). Slide-mounted cells were kept 16-24h at room temperature and stored at 4° C. in the dark. Cells were visualized and fluorescence intensity was quantified using fluorescence microscopy as described above. Intensity of fluorescence and occurrence of co-labeling were analyzed using the Metamorph 4.5 software (Universal Imaging Corporation, Canberra Packard Canada LTD, Mississauga, ON, CA. A 6-level (0 to 5) intensity scale was used to assess fluorescence intensities: background (level 0): 0-43 pixels, very weak (level 1); 44-85 pixels, weak (level 2); 86-128 pixels, moderate (level 3); 129-170 pixels, high (level 4); 171-213 pixels and very high (level 5): 214-255 pixels), as previously described.⁵⁸ Total fluorescence was determined for each image using arbitrary density units defined as: Σ (% cell labeled X intensity level). Levels 3-5 were considered as immunospecific.

Ribonuclease Protection Assay of Renal GHRH-R

Total RNA from medullas and purified descending and ascending thin limb cells was extracted with TRIzol (Invitrogen Canada, Burlington, ON). GHRH-R mRNA levels were assessed using the RPR64 probe corresponding to the 3′-end of the rat GHRH-R complementary DNA (cDNA) (nucleotide position: 1044-1611; Genbank accession number: L01407).² The ribonuclease protection assay was performed as previously described.³³ Twenty μg total RNA from renal medulla or purified thick HL or liver, 5 μg total RNA from purified thin limb cells or anterior pituitary were used. Tissue GHRH-R and GAPDH mRNA and cRNA external standard levels were quantified by densitometry. GHRH-R mRNA levels were always normalized with both GAPDH mRNA internal and GHRH-R cRNA external standards, in order to maintain an intra-assay coefficient of variation ≦10% in all experiments. Specificity of the [³²P]GHRH-R probe was assessed in each experiment using positive (5 μg pituitary total RNA) and negative (20 μg liver total RNA) controls. In addition, linearity of protected signals was assessed in each experiment, using 10-30 μg medulla total RNA. Results were expressed in percentage of relative density to the control condition or tissue preparation, using a fixed amount of total RNA, which reflects the concentration of GHRH-R mRNA at cellular level. Since changes may either be compensated or aggravated at the organ/tissue level, results were also expressed as total GHRH-R mRNA relative densities per renal medulla total RNA content, to document physiological impacts of interventions.³³

Northern Blot Hybridization of Anterior Pituitary GHRH-R

Total RNA was extracted as above. Northern blot hybridization was performed as previously described on 12 μg total RNA samples with minor modifications.³³ Prehybridization was performed in Robbins' hybridization solution (7% SDS containing 0.25 M Na₂HPO₄ (pH 7.4), 1 mM EDTA (pH 8.0) and 1% BSA) at 65° C., 2 h. Hybridization was performed in fresh Robbins' solution at 65° C. (ON), in the presence of labeled RPR64. Membranes were subsequently washed, exposed to films, stripped and rehybridized with GAPDH 28S probes.³³ Quantification of each GHRH-R mRNA transcript (2.5 and 4 kb), GAPDH mRNA and 28S rRNA levels was performed by densitometry. GHRH-R mRNA levels were normalized with 28S rRNA in all experiments, to maintain the intra-assay coefficient of variation ≦10%. Specificity of the [³²P]RPR64 cDNA probe was assessed in each experiment using 5 μg liver total RNA. Linearity of protected signals was measured routinely, using 6-18 μg total RNA. Results were expressed in percentage of relative density to that of control groups, using a fixed amount of total RNA. Results were also expressed as total GHRH-R mRNA relative densities per anterior pituitary total RNA content.

Reverse Transcriptase-PCR of preproGHRH

Total RNA from purified thin limb cells (2 μg) was subjected to two steps RT-PCR using SuperScript™ First-Strand Synthesis System (Invitrogen). Reverse transcription was performed using SuperScript™ II RT and PCR reaction was performed using Platinum® Taq DNA polymerase according to manufacturer's protocol (First-Strand synthesis using oligo(dT) PCR for targets up to 4 kb). PCR reaction was performed on a 1:5 dilution of the first strand cDNA product in a final volume of 50 μl containing 0.4 μl of Platinum® Taq DNA polymerase. Reagents were added to a final concentration of 1×PCR buffer [20 mM Tris-HCl (pH 8.4), 50 mM KCl], 1.5 mM MgCl₂, 0.2 mM dNTPs and 0.3 μM sense and antisense desalted primers diluted in sterile picopure water (GAPDH sense 5′-gggtgtgaaccacgagaaat-3′, GAPDH antisense 5′-actgtggtcatgagcccttc-3′, nt 1242-1376 GenBank NM_{—}017008; preproGHRH sense 5′-atgccactctgggtgttcttt-3′, preproGHRH antisense 5′-gcagtttgcgggcatataat-3′, nt 196-352 GenBank NM_{—}031577). The reaction was performed in Biometra TGradient PCR (Montreal Biotech Inc, Montreal, QC) with the following cycle profile: denaturation at 94° C. for 2 min, followed by 39 cycles of denaturation at 94° C. for 30 sec, annealing at 58° C. for 70 sec, extension at 72° C. for 60 sec and a final cycle at 94° C. for 30 sec, 58° C., 60° C., and 62° C. for 60 sec and a 5-min extension at 72° C. PreproGHRH and GAPDH PCR products were analyzed by gel electrophoresis on 2% agarose gel containing 0.5 μg/ml of ethidium bromide with a 100 bp molecular weight standard (Invitrogen).

RT-PCR of GHRH-R

Total RNA (2 μg) from purified aTL cells was subjected to two steps RT-PCR using SuperScript™ First-Strand Synthesis System for RT-PCR (Invitrogen/Canada Life Technologies). Reverse transcription was performed using SuperScript™ II RT and PCR reaction was performed using Platinum® Taq DNA polymerase according to manufacturer's protocol (First-Strand synthesis using oligo(dT) or GSP and PCR for targets up to 4 Kb). PCR reaction was performed on a 1:5 dilution of the first strand cDNA product in a final volume of 50 uL containing 0.4 ul of Platinum® Taq DNA polymerase. Reagents were added to a final concentration of 1×PCR buffer (20 mM Tris-HCl (pH 8.4), 50 mM KCl], 1.5 mM MgCl₂, 0.2 mM dNTPs) and 0.3 uM sense and antisense desalted primers from the rat (three sets of primers covering the 5′, middle portion and 3′ regions were used (PubMed NM_{—}012850): nt 58-191 (5′-ctctgcttgctgaacctgtg-3′ (sense (s)), 5′-catcccatggacgagttgtt-3′ (antisense (as)); nt 578-736 (5′-ctgctgtcttccagggtgat-3′ (s), 5′-taggagatgtggaggccaac-3′(as)); nt 1064-1227 (5′-acttcctgcctgacagtgct-3′ (s), 5′-tggcagaagttcagggtcat-3′ (as)), and porcine pituitary GHRH-R (three sets of primers three sets of rat primers covering the 5′, middle portion and 3′ regions were used (PubMed L11869: nt 144-271 (5′-ctgctgagctccctaccagt-3′ (s), 5′-cagcccgaggaggagttg-3′ (as)); nt 694-816 (5′-gcttctccacggttctgtgca-3′ (s), 5′-tgggtgacgtagaggccaag-3′(as)); nt 1201-1342 (5′-gctccttccagggcttcattgt-3′ (s), 5′-gaaggctttgcccatttggca-3′ (as)) cDNA sequence were diluted in sterile picopure water. The reaction was performed in Biometra TGradient PCR (Montreal Biotech Inc) with the following cycle profile: denaturation at 94° C. for 2 min, followed by 39 cycles of denaturation at 94.0 for 30 sec, annealing at 58.0° C., 60.0° C., and 62.0° C. for 70 sec, extension at 72° C. for 60 sec and a final cycle at 94° C. for 30 sec, 58.0° C., 60.0° C., and 62.0° C. for 60 sec and a 5-min extension at 72° C. GHRH-R, GAPDH and GHRH PCR products were analyzed by gel electrophoresis on 2% agarose gel containing 0.5 ug/ml of ethidium bromide with a 100 bp molecular weight standard (Invitrogen Life Technologies, Burlington, ONT, CA). The gel was visualized using a IS1000 Digital imaging system (Alpha Innotech Corp./Canberra Packard).

Quantitative Real-Time RT-PCR of GHRH-R and CICK-1

Total RNA from purified thin limb cells was extracted with TRIzol. Samples were resuspended in RNAse-free water (Ambion). Reverse transcription of 2 μg total RNA was performed using the SuperScript™ II RT kit (Invitrogen) and random hexamer primers, according to the manufacturer's protocol, including RNAse H treatement. mRNA levels of rat GHRH-R and rat GAPDH (internal control) were determined in separate tubes, by real-time PCR, using a 1/150 (GHRH-R) and 1/300 (GAPDH) dilution of the RT product and the reagents from the Quantitect™ SYBR® Green PCR kit (Qiagen, Mississauga, ON, CA), according to the manufacturer's recommendation. The ABI protocol was used except that the dUTP/uracil-N-glycosylase step was omitted. Reactions were performed in duplicate, in a final volume of 25 μL, containing 300 nM of sense and antisense primers, using a Rotor Gene 3000 real-time thermal cycler (Montreal Biotech Inc, Montreal, QC, CA). No template and no amplification controls were always included to confirm the specificity of reactions. The parameters included a single cycle of 95° C. for 15 min, followed by 45 cycles of 94° C. for 15 sec, annealing at 52° C. for 30 sec, extension at 72° C. for 30 sec and a melting step going from 72° C. to 99° C. (ramping at 1° C./sec). Specific primers (300 nM) for GHRH-R , CICk-1 and GAPDH were used. Specificity of the PCR products was established by melting curve analysis and by running products on 2% agarose gel, containing 0.5 μg/ml of ethidium bromide, with a 100 bp molecular weight standard (Invitrogen). Results were analyzed using the Rotor-Gene application software (version 6.0). A five-point standard curve was performed for each gene tested, using 1:5 serial dilutions (1:5 to 1:3125) of renal medulla total RNA from 2-month-old healthy male rats. The intra-assay coefficient of variation of GHRH-R and GAPDH Ct values was ≦2.5% in all experiments

Quantification of cAMP Levels in Semi-Purified Thin Limbs of Henle's Loop Cells

Sensitivity to GHRH was assessed in freshly semi-purified thin limb cells³³ from rats fed a 8%-NaCl diet for 2, 7 or 14 days or the control diet. Cells (1×10⁶ cells=60-75 μg prot/ml/Eppendorf tube) were preincubated 30 min (37° C.) in 1 ml DMEM/F12 cultured media,³³ containing 1× antibiotics, 0.2% BSA and 1 mM isobutyl-1-methylxanthine (IBMX, Sigma) and challenged 15 min (37° C.) with 1 and 100 nM GHRH, the vehicule (DMEM/F12-0.2% BSA) or 10 μM forskolin to assess the reactivity of cell preparation. The reaction was stopped by centrifugation (5 min, 4° C., 12 000 g). Pellets were resuspended in 200 μl of lysis buffer (10 min, RT, vortex) supplied with the EIA kit (cAMP Direct Biotrak™ enzyme immunoassay kit, Amersham Biosciences) and centrifuged (5 min, 4° C., 12 000 g). Supernatants were used to quantify immunoreactive cAMP levels (non-acetylated method). Pellets were kept frozen for determination of protein content.⁵⁹ Optical densities were measured at 450 nm, using a microplate reader (Bio-Rad, model 3550). The intra-assay coefficient of variation was ≦12% in all experiments. Net GHRH-induced cAMP levels (without basal level) were expressed in percentage of relative levels compared to that obtained in the presence of GHRH.

Quantification of Cell Proliferation in Semi-Purified Thin Limbs of Henle's Loop Cells

Freshly semi-purified cells were cultured in DMEM/F-12 culture media, containing antibiotics and the vehicle (culture medium) or 1, 10 or 100 nM rGHRH (1-29) NH₂. GHRH was added at time 0 and after a 24 and 48 h culture period. Proliferation was assessed with aliquots of 40,000 cells, after a 60-h cell culture period, using the Promega kit (CellTiter 96R Aqueous one solution cell proliferation assay).

Data Analysis and Statistics

RPA represents a more sensitive and reliable method to perform a valid quantification of GHRH-R mRNA levels in rat renal medulla and Henle's loop cells compared to Northern blotting.³³ However, Northern blotting was chosen to study the pituitary GHRH-R mRNA levels as it allows the detection of GHRH-R individual transcripts¹⁹. The validity of comparing GHRH-R mRNA levels, using RPA and Northern blotting was assessed using pituitary total RNA. In the pituitary from 3-day water-deprived rats, GHRH-R mRNA levels obtained from Northern blots (sum of densities of the two transcripts: 3.0±0.2 times higher than controls) were not significantly different from those obtained by RPA (sum of densities of the two protected fragments: 3.1±0.3 times higher than controls), indicating that medullary and pituitary GHRH-R mRNA levels can be compared. Quantification of GHRH-R mRNA transcripts, protected fragments and visualization of gels were performed using an IS1000 Digital imaging system (Alpha Innotech Corp/Canberra Packard, QC).

Results were expressed as mean±SEM. Comparisons of normalized GHRH-R mRNA levels as well as intracellular cAMP levels, immunofluorescence intensity, anti-BrdU immunoreactive cells were performed by ANOVA, followed by the Dunnett's multiple range test or by the unpaired Student's t test. Statistical significance of differences was established at P<0.05.

Rapid Amplification of GHRH-R cDNA Ends

The 5′- and 3′-Rapid Amplification of cDNA Ends (RACE)-Ready cDNAs were synthesized using the SMART RACE cDNA Amplification Kit, according to the manufacturer's recommendations (Clontech Laboratories Inc, Mountain View, Calif.). Briefly, the synthesis of cDNAs for 5′ RACE was performed in a final volume of 10 μl containing 1 μg of poly(A)⁺RNA provided from anterior pituitary, renal medulla or liver, 1.2 μM 5′-CDS (5′-(T)₂₅VN-3′, where N=A, C, G, or T and V=A, G, or C), 1.2 μM BD SMART II A oligonucleotide (5′-AAG CAG TGG TAT CAA CGC AGA GTA CGC GGG-3′), 1× first-strand buffer, 2 mM DTT, 1 mM each dNTP, and 1 μl BD Power-script reverse transcriptase. The synthesis of 3′-RACE cDNA was performed using the same strategy using 1.2 μM 3′-CDS Primer A (5′-AAG CAG TGG TAT CAA CGC AGA GTA C(T)₃₀VN-3′). The 5′- and 3′-RACE ready cDNAs were diluted 1:25 in 10 mM Tricine-KOH, pH 8.5, containing 1 mM EDTA and used in subsequent amplifications.

The primary PCR included 1 μl of 5′- or 3′-RACE-Ready cDNA (1:25), 1× universal primer mix (UPM) (long: 5′-CTA ATA CGA CTC ACT ATA GGG CAA GCA GTG GTA TCA ACG CAG AGT-3′; short: 5′-CTA ATA CGA CTC ACT ATA GGG C-3′) that recognized the SMART II oligonucleotide sequence, inserted in both 5′ and 3′ during cDNA synthesis, 0.2 μM rat GHRH-R gene-specific primer (Table 2: Ex-9/10P for 5′-RACE-Ready cDNA and Ex-7bP for 3′-RACE-Ready cDNA), 1× Advantage 2 PCR buffer, 0.2 mM each of dNTPs and 1× BD Advantage 2 Polymerase Mix containing AdvanTaq DNA polymerase, a proofreading polymerase, and TaqStart Antibody to provide automatic hot-start PCR (Clontech Laboratories Inc) in a total volume of 50 μl. All GHRH-R gene-specific primers used in PCR experiments (Table 2) were synthesized by Invitrogen Canada Inc. To improve the specificity of RACE, a touchdown PCR (Biometra instrument) was performed with the following cycle profile: 5 cycles of 94.0° C. (denaturation) for 30 sec and 72.0° C. (annealing and extension) for 3 min, 5 cycles of 94.0° C. for 30 sec, 70.0° C. (annealing) for 30 sec, and 72.0° C. (extension) for 3 min, followed by 20 cycles of 94.0° C. for 30 sec, 68.0° C. (annealing) for 30 sec, and 72.0° C. (extension) for 3 min. The primary PCR product was diluted 1:50 with Tricine/EDTA buffer. To further increase the specificity and sensitivity of amplification, secondary PCR was carried out with 5 μl or primary PCR product, 0.2 μM nested universal primer (NUP) (5′-AAG CAG TGG TAT CAA CGC AGA GT-3′), and 0.2 μM nested gene-specific primer (N) (Table 2: Ex-9P for 5′-RACE product and a primer complementary to Ex-7/8P for 390′-RACE product) in a total volume of 50 μl. The nested-PCR was performed using 20 cycles of denaturation at 94.0° C. for 30 sec, annealing at 68.0° C. for 30 sec and extension at 72.0° C. for 3 min.

TABLE 2
Sequence of GHRH-R gene-specific primers

| | | | Position on | | |
|---|---|---|---|---|---|
| | | | pituitary | Position on | |
| | | Sequence | GHRH-R | chromosome 4 | |
| ID | Application | (5′-3′) | cDNA | (AC091710) | Direction |
| Ex-1P | Characterization | Ctctgcttgct | 58-77 | 38112-38131 | S |
| | | gaacctgtg | | | |
| | | SEQ ID | | | |
| | | NO.: | | | |
| Ex-2/3P | Characterization | Catcccatg | 172-191 | 40807- | A |
| | | gacgagttgt | | 40822/40933- | S |
| | | t | | 40936* | |
| | | SEQ ID | | | |
| | | NO.: | | | |
| Ex-6P | Characterization/ | Ctgctgtcttc | 578-597 | 43668-43687 | S |
| | validation | cagggtgat | | | |
| | | SEQ ID | | | |
| | | NO.: | | | |
| Ex-7aP | Characterization | Taggagat | 717-736 | 45132-45151 | A |
| | | gtggaggcc | | | S |
| | | aac | | | |
| | | SEQ ID | | | |
| | | NO.: | | | |
| Ex-11P | Characterization/ | Acttcctgcc | 1064-1083 | 47288-47307 | S |
| | sequencing | tgacagtgct | | | |
| | | SEQ ID | | | |
| | | NO.: | | | |
| Ex-13P | Characterization | Tggcagaa | 1208-1227 | 50611-50630 | A |
| | | gttcagggtc | | | S |
| | | at | | | |
| | | SEQ ID | | | |
| | | NO.: | | | |
| Ex- | 5′RACE | Cagcccaa | 924-897 | 46016- | A |
| 9/10P | | agttcacccc | | 46028/46594- | S |
| | | aacagaga | | 46608* | |
| | | gg | | | |
| | | SEQ ID | | | |
| | | NO.: | | | |
| Ex-9P | 5′RACE (nested) | Ccagtagg | 873-847 | 45992-45966 | A |
| | | gggagctgt | | | S |
| | | cgtctaggtc | | | |
| | | SEQ ID | | | |
| | | NO.: | | | |
| Ex-7bP | 3′RACE | Ctggtggct | 753-777 | 45168-45192 | S |
| | | ggttctcgct | | | |
| | | ggctgg | | | |
| | | SEQ ID | | | |
| | | NO.: | | | |
| Ex-7/8P | 3′RACE (nested) | Tctcgctgg | 765-789 | 45180- | S |
| | | ctggggact | | 45193/45480- | |
| | | ccctgtg | | 45490 | |
| | | SEQ ID | | | |
| | | NO.: | | | |
| Ex-8P | Sequencing | Cctgtgctat | 824-842 | 45485-45503 | S |
| | | gcactggta | | | |
| | | SEQ ID | | | |
| | | NO.: | | | |
| Ex-1M | Validation | Agacacgg | — | 18647-18665 | S |
| | | gaccagaa | | | |
| | | atc | | | |
| | | SEQ ID | | | |
| | | NO.: | | | |
| Ex-2M | Validation | Agcctttatc | — | 19331-19350 | S |
| | | catgcgaca | | | |
| | | g | | | |
| | | SEQ ID | | | |
| | | NO.: | | | |
| Ex-11M | Validation | Gatgcaaat | 945-922 | 46606-46629 | A |
| | | tatattgaga | | | S |
| | | aacag | | | |
| | | SEQ ID | | | |
| | | NO.: | | | |

Primers were identified according to their exon localization on the rat anterior pituitary
(P) (Genebank NM_012850) or renal medulla (M) GHRH-R cDNA.
*Primer sequence localized on two adjacent exons and separated on chromosome 4 by an intronic sequence.
S: sense. AS: antisense.

Purification and Sequencing

The renal medulla and anterior pituitary 5′-RACE products were analyzed by electrophoresis on a 1% agarose gel, using TAE buffer, containing 0.5 μg/ml ethidium bromide and visualized. Each fragment was eluted using the MinElute Gel Extraction Kit (Qiagen, Mississauga, ON, CA). Purified products were inserted into the pCR4-TOPO vector and amplified in TOP10 E. Coli (Topo TA cloning kit, Invitrogen Inc). Amplified plasmids were purified using the Quick Lyse MiniPrep kit (Qiagen). The insert in the plasmid was sequenced using the primer pair supplied with the cloning kit (M13 forward: 5′-gtaaaacgacggccag-3′; M13 reverse: 5′-caggaaacagctatgac-3′). The renal medulla and anterior pituitary 3′-RACE products, pooled from three PCR reactions, were purified on 1% agarose gel containing 0.5 μg/ml ethidium bromide, visualized and extracted using the MinElute Gel Extraction Kit (Qiagen). The purity of each PCR product was ascertained by gel electroproresis as above, and PCR products were sequenced using specific GHRH-R primers (Table 2: Ex-8P and Ex-11P). Sequencing was performed at the CHUM Research Center core facilities, using AmpliTaq DNA polymerase FS and a 16-capillary ABI Prism 3100 fluorescent sequencer (Applied Biosystems Canada/Ambion).

General Characteristics of Rats Submitted to a High-NaCl Diet or Water Deprivation

Body weight (BW), food and water intakes, urine flow rate and urine sodium rate of rats fed a 8%-NaCl diet (GHRH-R mRNA study), and BW and food intake of water-deprived rats are reported in Table 3. As previously observed,^{35, 36} water intake, urine flow rate and urine sodium excretion rate of rats submitted to the 8%-NaCl diet were significantly increased when compared to controls (P<0.001). After 14-day of the high-salt regimen, BW was decreased by 7% (P<0.01). Food intake was not modified by this diet. No change was observed in BW and food intake of 2-month-old rats submitted to a 2-day 8%-NaCl diet and either injected with GHRH or saline. BW of water-deprived rats was decreased when compared to controls (P<0.001), as reported before.³⁷ Moreover, their food intake decreased (P<0.001), providing an explanation for the loss of BW.

TABLE 3
Characteristics of rats following a 2-, 7-, or 14-day high-NaCl diet or a 3- or 5-day water deprivation

| | | High- | | High- | | High- | | Water | | Water |
|---|---|---|---|---|---|---|---|---|---|---|
| | Control | NaCl | Control | NaCl | Control | NaCl | Control | deprivation | Control | deprivation |
| Duration (day) | 2 | 2 | 7 | 7 | 14 | 14 | 3 | 3 | 5 | 5 |
| Number of animals | 8 | 7 | 8 | 8 | 8 | 8 | 8 | 9 | 8 | 9 |
| Body weight (g) | 264 ± 5 | 266 ± 3 | 323 ± 11 | 315 ± 14 | 382 ± 5 | 354 ± 7^{a} | 242 ± 2 | 172 ± 2^{b} | 343 ± 8 | 268 ± 6^{b} |
| Food intake (g/day) | 25 ± 2 | 23 ± 1 | 25 ± 1 | 26 ± 1 | 25 ± 1 | 27 ± 2 | 22 ± 2 | 8 ± 2^{b} | 23 ± 1 | 5 ± 1^{b} |
| Water intake | 26 ± 2 | 89 ± 6^{b} | 32 ± 4 | 109 ± 7^{b} | 41 ± 2 | 116 ± 7^{b} | 34 ± 2 | 0 | 29 ± 3 | 0 |
| (ml/day) | | | | | | | | | | |
| Urine flow rate | 5.5 ± 0.7 | 29.8 ± 2.5^{b} | 4.0 ± 0.9 | 26.5 ± 1.6^{b} | 5.4 ± 0.8 | 25.9 ± 1.8^{b} | ND | ND | ND | ND |
| (ml/day/100 g BW) | | | | | | | | | | |
| Urine_{Na} rate | ND | ND | 57.6 ± 7.0 | >300^{b} | ND | ND | ND | ND | ND | ND |
| (mmol/day/l) | | | | | | | | | | |

Values are means ± SEM of the data compiled at the end of the study.
High-NaCl: 8%-NaCl chow.
Control: 0.3%-NaCl chow.
ND: not determined.
Values represent the mean ± SEM.
^{a}P < 0.01 and
^{b}P < 0.001, compared to respective controls, by two-tailed unpaired Student's t test.

### Example 2

### GHRH-R Expression Profile

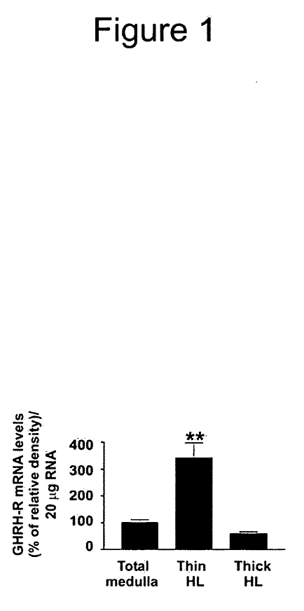

To increase knowledge on the medullary GHRH-R and identify its cell-specific localization, purified thin and thick limbs of Henle's loop cells were prepared. GHRH-R mRNA levels were analyzed by ribonuclease protection assay (RPA) as described in Example 1.³³ Two distinct bands were detected, using the RPR64 rGHRH-R probe and their sum was considered as the total level of GHRH-R mRNA, as in previous works.^{33, 38} In kidneys from 2-month-old healthy male rats (FIG. 1), thin limb cells contained highest levels of GHRH-R mRNA. A high level of GHRH-R mRNA was detected in thin limbs only. Those found in ascending thick limb (ATL) cells and total medulla were 5.8 and 3.4 times lower, respectively (P<0.01). GHRH-R mRNA levels from ATL cells were 1.7 times lower than those in total medulla (P<0.05).

Immunocytochemical Localization of GHRH-R in Thin Limbs of Henle's Loop

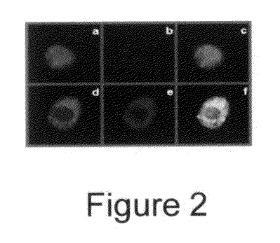

As the highest level of GHRH-R mRNA was observed in thin limbs of Henle's loop (HL) cells, a purified cell preparation was used to assess the precise localization of GHRH-R. Since thin limb cells contains a descending segment participating to water transport, and an ascending segment actively involved in ion transport, it was important to identify the specific cell type expressing GHRH-R in this part of the nephron, to help defining potential roles. Co-immunolocalization of GHRH-R, with specific markers of descending (aquaporin-1)⁴¹ and ascending (CIC-K1)^{36, 42} thin limb cells was performed. Co-immunolocalization of GHRH-R with markers of the thin descending (aquaporin-1) and thin ascending (CIC-K) limbs of HL cells revealed as shown in FIG. 2 that aquaporin-1 positive cells (FIG. 2a) were devoid of GHRH-R (FIGS. 2b, 2c). However, CIC-K (FIG. 2d) and GHRH-R co-labeling was observed in of the cells (FIGS. 2e, 2f). No signal was seen when the GHRH-R or CIC-K antibody was substituted by normal IgGs (data not shown).

Immunocytochemical Localization and Gene Expression of GHRH in Thin Limbs of Henle's Loop

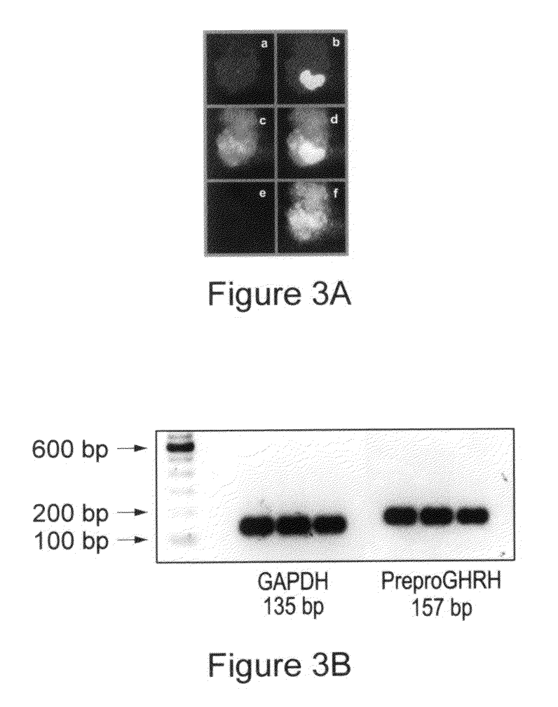

GHRH and CIC-K immunofluorescence was always co-localized as shown in FIG. 3. No signal was observed when the GHRH primary antibody was substituted by normal IgGs (FIG. 3A panel e). The GHRH fluorescent signal overlapped ≈45% of CIC-K immunoreactive cells (FIG. 3A panel f). In addition, positive results from RT-PCR strongly suggest that immunoreactive preproGHRH is locally synthesized in thin limbs of HL (FIG. 3B). In conclusion, GHRH-R immunofluorescence was highly co-localized with that of CIC-K but not aquaporin-1, indicating a specific expression in ascending thin limb cells. Moreover, these results show, for the first time, the presence of a local GHRH-GHRH-R system in these cells.

### Example 3

### In Vivo Regulation of Renal Medulla GHRH-R mRNA Levels Following a 2-, 7- or 14-Day High-NaCl Diet or a 3- or 5-Day Water Deprivation

To gain more information on potential roles of the renal GHRH-R, the regulation of medullary GHRH-R mRNA levels was studied using in vivo models of renal dysfunction by Na⁺/C⁻ or water homeostasis disruption, as obtained with a high-NaCl diet for 2, 7 or 14 days or a water deprivation for 3 or 5 days.

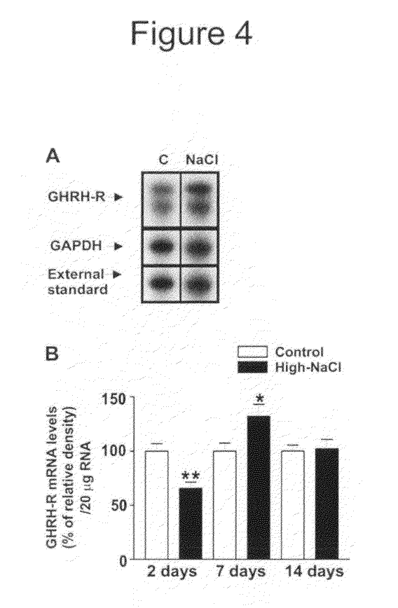

At the cellular level (GHRH-R mRNA level per fixed amount of total RNA), GHRH-R mRNA concentrations were differentially regulated according to the duration of the high-salt diet. They decreased at 2 days, increased at 7 days, and returned to normal at 14 days. This is shown in FIG. 4, wherein after a 2- and 7-day 8%-NaCl dietary intake, renal medulla GHRH-R mRNA levels were 1.4-fold lower (P<0.01) and 1.3-fold higher (P<0.05) than those of control rats (0.3% NaCl), respectively, when expressed per 20 μg total RNA, to reflect cellular levels.

The same type of change was seen when data were analyzed at the tissue level (GHRH-R mRNA levels per total medulla RNA content), indicating that the cellular effect was not counterbalanced systemically. Indeed, they were decreased by 1.5-fold after 2 days (P<0.05) and increased by 1.7-fold after 7 days (P<0.01) of the 8%-NaCl diet, when expressed per medulla total RNA content, to reflect tissue level (data not shown).

After 14 days of the regimen, no significant difference was observed between GHRH-R mRNA levels from rats submitted to the high-salt diet and controls, either expressed per 20 μg total RNA (FIG. 4 B) or per medulla total RNA content (data not shown). The effects of the high-NaCl diet were not mimicked by a 3- or 5-day water deprivation, two situations provoking hypertonicity. After 3- or a 5-day water deprivation, no significant difference was observed between GHRH-R mRNA levels from water-deprived and control rats, having free access to water (data not shown). Therefore, this indicates that the ascending thin limb GHRH/GHRH-R system is not directly involved in the regulation of ion transport.

High-salt diet regulates genes involved in higher fibrotic activity, cellular stress and apoptotis in the rat renal medulla.⁴⁶ and administration of substances exhibiting antioxidant properties attenuates or prevents these deleterious effects.^{47, 48} Changes in GHRH-R mRNA levels and GHRH sensitivity, between 2 and 7 days of a high-NaCl diet, suggests that GHRH-R activation may promote ascending thin limb cell survival early on in a situation of oxidative stress and subsequently proliferation. The GHRH-R could, rather than regulate ion transport, participate to adaptive processes in ascending thin limb cells to compensate for an increased oxidative stress and cell damage caused by a drastic and sustained high-NaCl intake.⁴⁵

### Example 4

### In Vivo Regulation of Anterior Pituitary GHRH-R mRNA Levels Following a 2-, 7- or 14-day high-NaCl Diet or a 3- or 5-Day Water Deprivation

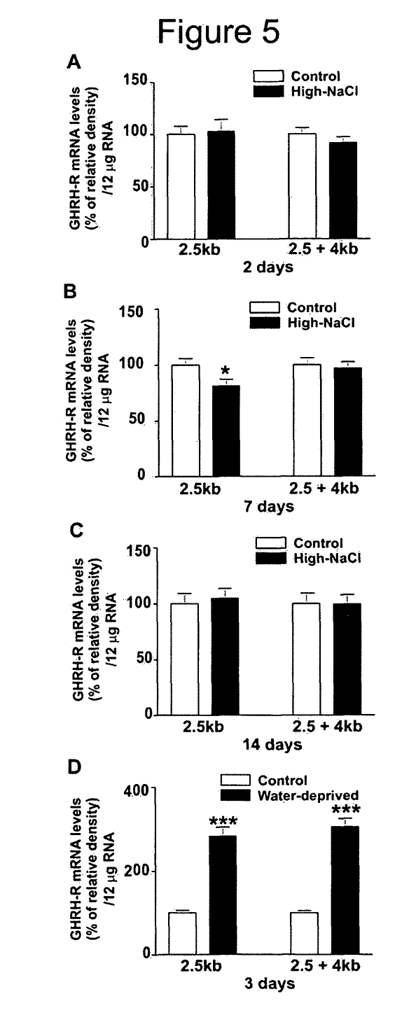

The presence of 2.5- and 4-kb GHRH-R mRNA transcripts was observed in the anterior pituitary of all rats (controls, 8%-NaCl-fed, water-deprived), as previously reported.¹⁰ In the pituitary from high-salt-fed rats, no drastic changes of GHRH-R mRNA transcript levels were observed when expressed per 12 μg total RNA (FIG. 5A-C). After 7 days of the regimen, the levels of the 2.5-kb GHRH-R mRNA transcript was transiently decreased by 1.2-fold (P<0.05; FIG. 5B). No change in pituitary GHRH-R mRNA levels was observed, at any time, when data were expressed per pituitary total RNA content (data not shown).

After 3 days of water deprivation, pituitary levels of the 2.5-kb GHRH-R mRNA transcript and combined levels of 2.5-kb and 4-kb transcripts, increased 2.8- and 3.0-fold (P<0.001), respectively, when expressed per 12 μg total RNA (FIG. 5D). When GHRH-R mRNA levels were analyzed per anterior pituitary total RNA content, levels of the 2.5-kb transcript and combined levels of 2.5-kb and 4-kb transcripts increased 1.5-fold (P<0.05) (data not shown). After 5 days of water deprivation, pituitary levels of the 2.5-kb GHRH-R mRNA transcript and combined levels of 2.5-kb and 4-kb transcripts, when expressed per 12 μg total RNA, increased 1.8- and 1.9-fold (P<0.001), respectively (data not shown). When GHRH-R mRNA levels were analyzed per anterior pituitary total RNA content, levels of the 2.5-kb transcript and combined levels of 2.5-kb and 4-kb transcripts were increased 1.3- (P<0.05) and 1.4-fold (P<0.01), respectively (data not shown).

As such, the pituitary GHRH-R, which is exclusively localized on somatotroph cells,⁵² was found to be insensitive to the high-salt diet, contrarily to that of ascending thin limbs, demonstrating the vulnerability of the latter.

A tissue-specific regulation of renal medulla and anterior pituitary GHRH-R mRNA levels has previously been shown in developing and aging rat.³³ Whether or not somatotroph sensitivity to GHRH can be altered during the first 2 days of the high-NaCl diet, without affecting GHRH-R mRNA levels, remains possible. In contrast, a water deprivation strongly increased pituitary cell and tissue GHRH-R mRNA levels. Since it induces a drastic reduction of food intake and that dietary protein restriction down-regulates hypothalamic preproGHRH mRNA,⁵³ a subsequent decrease of pituitary GHRH-R may have occurred. These results suggest that somatotroph and ascending thin limb cell GHRH-R mRNA levels may be primarily regulated by hypothalamic and renal GHRH, respectively.

### Example 5

### Sensitivity to GHRH in Semi-Purified Thin Limbs of Henle's Loop Cells from Rats Submitted to a 2-, 7- or 14-Day High-NaCl Diet

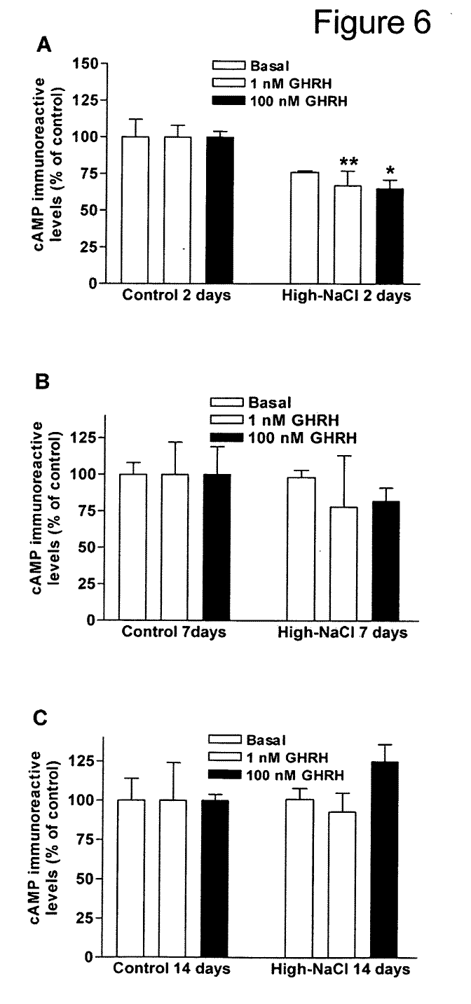

Sensitivity to GHRH in thin limbs of Henle's loop cells from rats submitted to a 2-, 7- or 14-day high-NaCl diet was assessed by measuring GHRH-induced intracellular cAMP production, in freshly dispersed semi-purified thin limb cells as shown in FIG. 6. Basal or forskolin levels of immunoreactive cAMP were not significantly decreased in rats fed 2 days with 8%-NaCl chow, although a trend was observed. Sensitivity to rGHRH(1-29)NH₂ was altered and GHRH-induced cAMP production was decreased 1.5-fold (1 nM: P<0.01; 100 nM: P<0.05) (FIG. 6A). This loss of sensitivity to GHRH was reverted in rats fed the high-salt diet for 7 or 14 days (FIGS. 6B, 6C).

The regulation of GHRH-R mRNA levels was therefore reflected in the sensitivity of GHRH to induce cAMP production in freshly dispersed thin limb cells from rats submitted to the high-NaCl diet, in comparison to those fed the control diet. After 2 days of high-salt diet, a stimulation with either a low (1 nM) or high (100 nM) concentration of GHRH resulted in a decreased production of cAMP, correlating with that of GHRH-R mRNA levels. After 7 days, GHRH-induced cAMP levels were restored, indicating that an increased production of GHRH-R mRNA may be necessary to rapidly restore GHRH sensitivity and likely GHRH-R functional receptor levels. Data on the effect of high-salt-induced oxidative stress in thin limb cells specifically is unique to this study.

### Example 6

### In Vivo Effect of a GHRH Treatment on DNA Repair/Synthesis in Purified Thin Limbs of Henle's Loop Cells from Rats Submitted to a 2-Day High-NaCl Diet

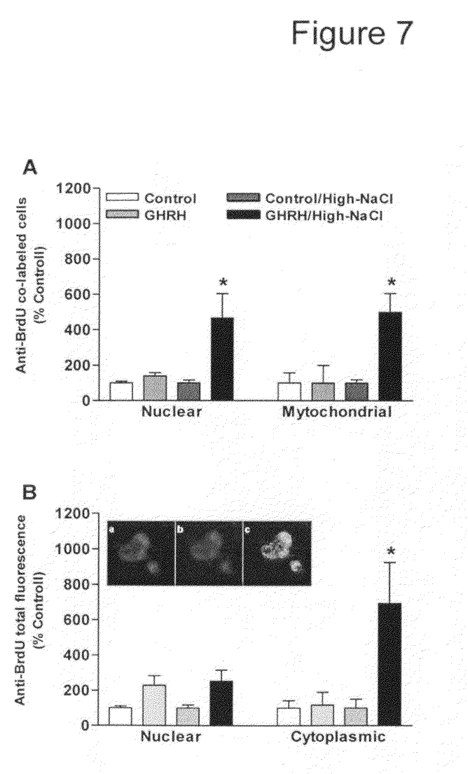

As shown in FIG. 7, when 2-month-old rats fed 2 days to a 8%-NaCl chow were injected daily with GHRH (1 mg/kg BW sc/day), a 5 times increase in the number of ascending thin limb cell nuclei and mitochondria immunolabeled to BrdU was observed in thin limbs of Henle's loop cells (P<0.05), when compared to various control groups (normal diet, with or without GHRH injections, high-salt diet alone). Moreover, the intensity of mitochondrial BrdU immunofluorescence was increased ≈7 times in these cells (P<0.05). GHRH-R mRNA levels tended to increase in the renal medulla of the high-NaCl fed rats, injected with GHRH and serum total insulin-like growth factor-1 (IGF-1) levels were not modified (data not shown). A subcutaneous (sc) administration of GHRH, once a day from the beginning of a 2-day high-NaCl diet, increased markedly the number of ascending thin cell nuclei and mitochondria immunolabeled to BrdU. In addition, the intensity of anti-BrdU labeling was significantly augmented in the cytoplasm co-labeling with MitoTracker red CMXRos, a reliable indicator of functional mitochondria.

Thus, in condition of oxidative stress, activation of the renal GHRH-R plays a role in adaptive processes related to DNA repair and/or synthesis, leading to cell survival and subsequent proliferation of these squamous epithelial cells not very rich in mitochondria.

### Example 7

### In Vivo Effect of a GHRH Treatment on GHRH-R and CICk-1 mRNA Levels in Purified Thin Limb Cells from Rats Submitted to a 2-Day High-NaCl Diet

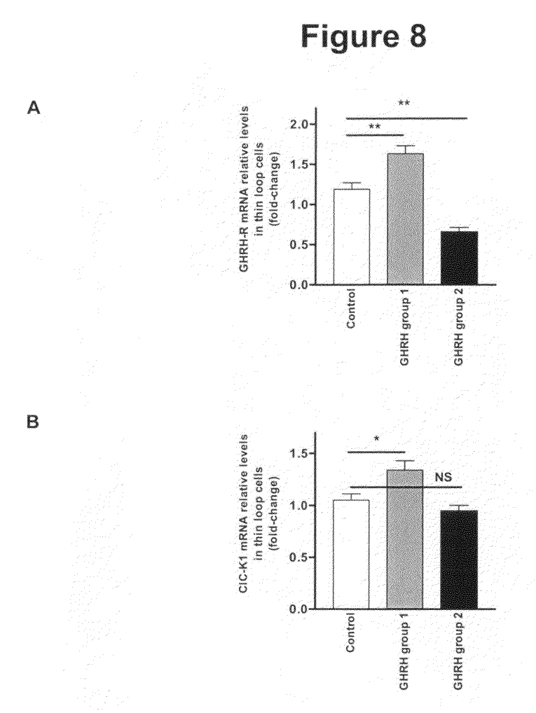

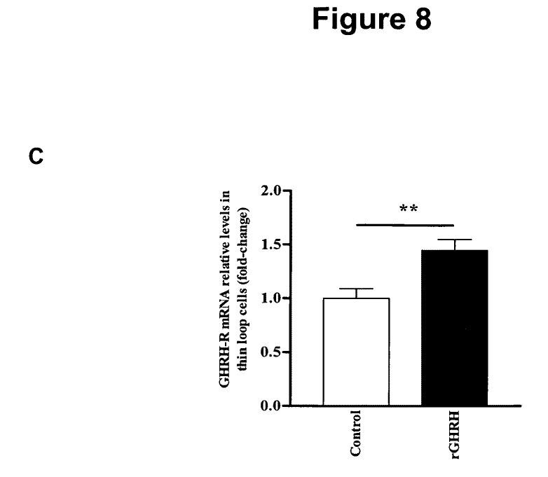

The in vivo effect of GHRH administration on GHRH-R and CICK-1 mRNA levels (measured by real-time RT-PCR) in purified thin limb cells from rats submitted to a 2-day high-NaCl diet was studied. GHRH-R (FIG. 8A) and CICK-1 (FIG. 8B) mRNA levels were significantly increased in the total renal medulla of a subgroup of 3 rats, essential in ascending thin limb functions. GHRH-R mRNA levels were decreased without significantly altering those of CIC-K1 in 5 others. Regulation of GHRH-R mRNA levels in thin limb cells of 2-month-old healthy male Sprague Dawley rats eas also studied following a subcutaneous administration of rGHRH(1-29)NH2 (0.5 mg/kg BW/day) or the saline vehicle, for 2 days. Six animals were used in each treatment. **P<0.01 (FIG. 8C) These results indicate that a lower GHRH dosage, such as 0.5 mg/kg BW/day, will up-regulate the renal GHRH-R in a large number of rats.

As discussed above, GHRH directly induces thin limb cell proliferation in vitro. No significant regulatory effect was seen on anterior pituitary GHRH-R mRNA levels with a 2-day in vivo of GHRH (data not shown). It was previously shown that IGF-I serum levels are significantly decreased after a 14-day sc administration of 1 mg/kg BW/day rGHRH(1-29)NH₂ but not with 0.5 mg/kg BW/day).¹⁰ Therefore, the dosage and duration used to regulate the renal GHRH-R will not regulate the pituitary GHRH-R.

### Example 9

### In Vivo Effect of a GHRH Treatment on Cell Proliferation in Purified Thin Limb Cells from Normal Rats

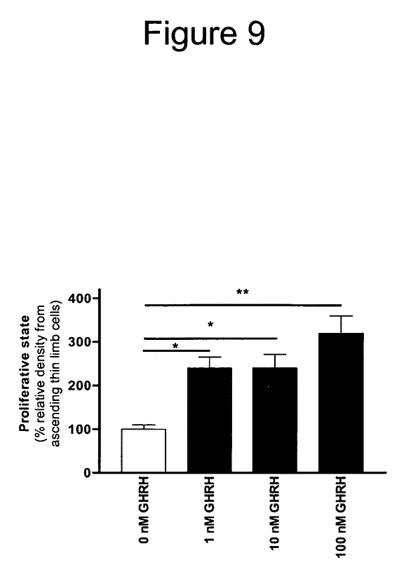

The effect of GHRH on proliferation was directly assessed in semi-purified thin limbs of Henle's loop cells from healthy 2-month-old rats. As shown in FIG. 9, rGHRH(1-29)NH₂ induced a 2.4 to 3.2-fold increase of the proliferative index in these cells (1 and 10 nM: P<0.05; 100 nM: P<0.01) when compared to control cell stimulated with the GHRH vehicle.

### Example 8

### RT-PCR Products from Rat and Porcine Anterior Pituitary and Renal Medulla, Using a Panel of Anterior Pituitary GHRH-R Primers

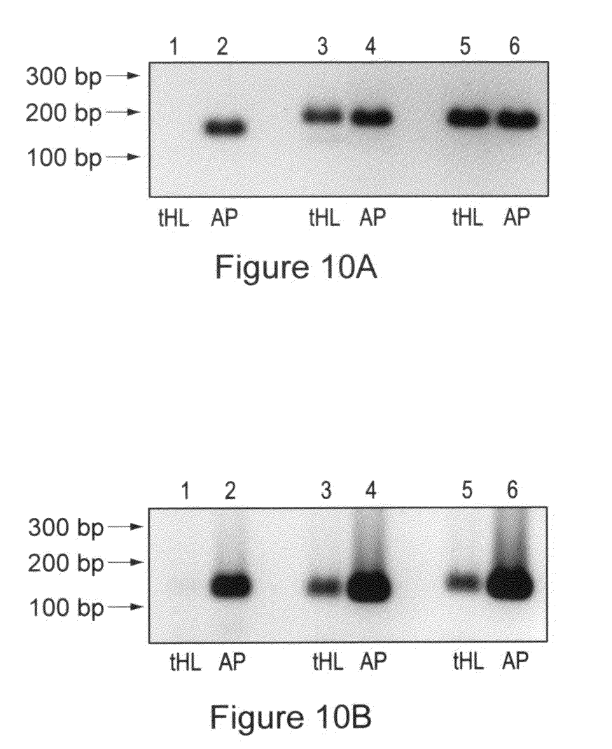

Using a panel of primers targeting the 5′ end, median portion and 3′ end of the pituitary GHRH-R, RT-PCR products from rat and porcine anterior pituitary and renal medulla were studied. A similar pattern was observed in both the rat and porcine renal medulla in comparison with anterior pituitary. No signal was detected in the rat (FIG. 10A) and porcine (FIG. 10B) medulla when 5′ end primers were used.

Difference in the primary structure of the pituitary and renal GHRH-R and/or the relative abundance of the native 423-aa GHRH-R and isoforms may contribute to a tissue-specific regulation. In the rat pituitary, apart from the 423-aa GHRH-R, two splice variants have been identified^{2, 11, 54} but their relative abundance has not been quantified rigorously. The 464-aa variant bears a 41-aa addition inserted into the 3^{rd} IC domain,¹¹ while the 480-aa variant bears the long 3^{rd} IC loop and a modified C-terminus, resulting from a 131-bp deletion (nt 1279-1408).⁵⁴ GHRH binds with moderate affinity to the 464-aa variant, transiently transfected in HeLa cells, and induces⁵⁵ or not¹¹ cAMP production. The ability of GHRH to stimulate cAMP was reported to be lower with the 480-aa variant than the 464-aa variant,⁵⁴ suggesting that the^{3rd} IC loop and the C-terminus are critical for GHRH-activation of the cAMP-AC-PKA pathway.

Action of GHRH is mediated in rat ascending thin limb cells by a GHRH-R exhibiting a 5′ DNA sequence different from that of the rat anterior pituitary GHRH-R. This structural difference in the rat renal GHRH-R compared to the pituitary GHRH-R was also observed for the murine (data not shown) and porcine renal GHRH-R. As porcine and human pituitary GHRH-R share the highest sequence identity (86%),^{2, 3} it is suggested that the renal GHRH-R variant found in the rat medulla is also present in human renal medulla.

### Example 10

### Identification of Renal Medulla GHRH Receptor from Rat

Considering the difference in the 5′ coding region compared to that of anterior pituitary and to obtain information on the renal GHRH-R cDNA sequence, a 5′ RACE was performed with the SMART RACE cDNA Amplification kit (Clontech) as described in Example 1. It allowed generating a high yield of full-length, double-stranded cDNA from small amounts of starting RNA and contains all reagents to synthesize cDNA and perform 5′ and 3′ amplification reactions from the same template.

Although structure difference between the renal and pituitary GHRH-R in the 3′ coding region were not expected, 3′ RACE was performed as a positive control. When needed, nested primers were used. Total RNA from renal medulla of 2-month-old male SD rats was extracted with TRIzol. Poly (A) mRNA was used after purification on Oligotex resin (Qiagen). The purity and integrity of total RNA was analyzed using an Agilent 2100 Bioanalyser (Agilent Technologies). The specific primers selected hybridized to the central region of the pituitary receptor cDNA and exhibit the various physico-chemical characteristics required in this approach. The amplified sequences in the 5′ and 3′ RACE strategies overlaped each other in a tract of 172 nucleotides, to facilitate sequence analysis.

Amplification products were resolved on agarose gel electrophoresis and isolated with NucleoTrap Gel Extraction kit, provided with the SMART RACE system, quantified and quality-controlled by electrophoresis. Since the proofreading activity of the polymerase removes the 3′ overhangs during PCR and makes TA cloning very inefficient, fragments were treated with Taq Polymerase, after gel extraction to add the 3′A overhangs necessary for the TA cloning. DNA fragments were directly cloned into the pCR4-TOPO vector (Invitrogen) and the vector was amplified using E. coli Top10 (Invitrogen). Cloned inserts were sequenced.

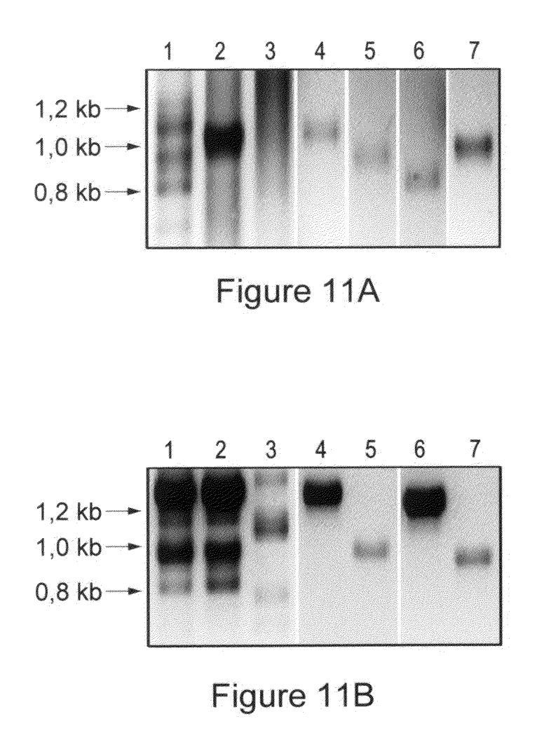

The 5′ RACE gave three products of 1.1, 0.9 and 0.8 kb from renal medulla Poly(A)⁺RNA, using UPM/Ex-9/10P and NUP/Ex-9P primer pairs (FIG. 11A, lane 1). A single product of 1.0 kb (expected size: 0.93 kb) was obtained from anterior pituitary Poly(A)⁺RNA (positive control) (FIG. 11A, lane 2), and no signal was obtained with liver RNA (negative control) (FIG. 2A, lane 3). Following purification (FIG. 11A, lanes 4-7), insertion of individual products in the plasmid pCR4—TOPO, transformation of TOP10 E. coli colonies, and plasmid amplification, 4, 7 and 10 sequences were analyzed for the 1.1-, 0.9- and 0.8-kb renal GHRH-R cDNA fragments, respectively, as well as three sequences for the 1.0-kb pituitary GHRH-R cDNA fragment.

The 3′ RACE gave two major products of 1.2 and 0.9 kb (expected size: 0.92 kb), exhibiting no apparent size difference between renal medulla and anterior pituitary Poly(A)⁺RNA (FIG. 11B, lanes 1-2). The size of low intensity products obtained using liver RNA was not compatible with that of a GHRH-R entity (FIG. 11B, lane 3). Each renal medulla and anterior pituitary PCR product obtained was homogeneous after gel extraction (FIG. 11B, lanes 4-7), and four sequences were obtained for each one.

GHRH-R Sequence Analysis

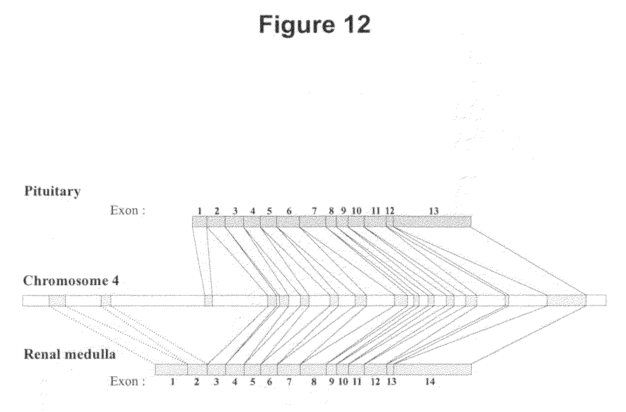

When the renal medulla 5′-RACE 1.1-kb product was aligned with 3′-RACE 1.2-kb renal/pituitary product, the resulting GHRH-R cDNA sequence was homologous with that of anterior pituitary, from the beginning of exon 2 to the stop codon. Upstream of exon 2, the renal medulla GHRH-R sequence did not match that of anterior pituitary GHRH-R exon 1 or intron 1, excluding the possibility of alternative splicing in intron 1. The 306-bp novel sequence, corresponding to a different region on chromosome 4, was located at approximately 19,000 bp upstream of the anterior pituitary GHRH-R transcription start site (FIG. 12). When compared to the genomic DNA sequence (AC091710), it was shown to contain two exons, with splice donors and acceptors in intronic sequences (FIG. 12). They were named exon 1M and 2M for the renal medulla GHRH-R exon 1 and 2, respectively. When the anterior pituitary 5′-RACE 1.0-kb product was aligned with 3′-RACE 1.2-kb renal/pituitary product, the resulting GHRH-R cDNA sequence was completely homologous with the 1269-bp pituitary sequence. The renal medulla and anterior pituitary GHRH-R cDNAs contained all splice donor and acceptor sequences to form a 14- and 13-exon sequence, respectively (FIG. 12). The osition of exons of the anterior pituitary and renal GHRH-R on the genomic sequence AC091710 is shown in Table 4.

TABLE 4
Position of exons of the anterior pituitary and
renal GHRH-R on the genomic sequence AC091710

| | Anterior pituitary | Renal medulla |
|---|---|---|
| | sequence | sequence |
| Exon A/Exon 1M | | 18576-18768 |
| Exon B/Exon 2M | | 19299-19411 |
| Exon 1 | 38055-38138 | |
| Exon 2/Exon 3M | 40720-40822 | 40720-40822 |
| Exon 3/Exon 4M | 40933-41040 | 40933-41040 |
| Exon 4/Exon 5M | 41592-41689 | 41592-41689 |
| Exon 5/Exon 6M | 42686-42783 | 42686-42783 |
| Exon 6/Exon 7M | 43582-43714 | 43582-43714 |
| Exon 7/Exon 8M | 45040-45193 | 45040-45193 |
| Exon 8/Exon 9M | 45480-45540 | 45480-45540 |
| Exon 9/Exon 10M | 45959-46028 | 45959-46028 |
| Exon 10/Exon 11M | 46594-46685 | 46594-46685 |
| Exon 11/Exon 12M | 47226-47355 | 47226-47355 |
| Exon 12/Exon 13M | 48709-48750 | 48709-48750 |
| Exon 13/Exon 14M | 50577-51033 | 50577-51033 |

The loss of the pituitary exon 1 in the renal GHRH-R cDNA sequence leads to a loss of the initiation of translation codon described in the anterior pituitary GHRH-R sequence. No start ATG was found in exon 1M or 2M. However, a Kozak consensus-like sequence (tgctccATGG instead of gcca/gccATGG) is present in exon 3M (exon 2 in the pituitary GHRH-R sequence), with a 1113-bp open reading frame (ORF). This ORF will lead to a 371-aa deduced protein instead of the 423-aa anterior pituitary GHRH-R, identical to the portion 53-423 of the pituitary GHRH-R and lacking the first 52 aa. This aa sequence retains the seven TM domains, five out of the six Cys conserved in the 423-aa GHRH-R sequence, and a crucial Asp in position 8 (Asp⁶⁰ in the pituitary GHRH-R). However the renal GHRH-R would lack the Asn in position 50 of the pituitary GHRH-R, a site for potential glycosylation.

Sequencing of the renal medulla 5′-RACE 0.9-kb product (FIG. 11A, lanes 1, 5) revealed the presence of two entities. The first one exhibited the same structure than the 1.1-kb product described above, while lacking exon 8M (pituitary GHRH-R exon 7). The absence of exon 8M, due to alternative splicing would change the ORF, leading to the insertion of a premature stop codon in exon 10M. The putative 171-aa truncated GHRH-R (aa sequence 53-199 of the pituitary GHRH-R) would only have four TM. The last 24 aa of this truncated receptor having no homology with the pituitary GHRH-R. The second product corresponded to the 1.1-kb sequence, but lacking exon 1M. Interestingly, it would lead to the 371-aa GHRH-R described above, with the start codon in exon 3M.

Analysis of the renal medulla 0.8-kb 5′-RACE product (FIG. 11A, lanes 1, 6), led to the identification of three GHRH-R cDNA sequences. The first and second one began with exon 3M and 4M, respectively. The deduced aa sequences generated would be the 371-aa GHRH-R already described and a 227-aa product (aa sequence 197-423 of the pituitary GHRH-R), respectively. Finally, the first two exons of the third sequence had no homology with either the anterior pituitary GHRH-R exon 1 or the renal GHRH-R exon 1M or 2M. This sequence corresponded to a genomic sequence, located at approximately 15,500 bp upstream of the pituitary initiation transcription site on chromosome 4. When this sequence was present, exons 1M to 6M and 8M were absent, leading to a putative 33-aa peptide devoid of homology with any known protein from the rat genome.

The 21 sequences obtained from the 5′-RACE products indicates that at least 52% of the cDNA sequences (11/21) would lead to the 371-aa GHRH-R, while 14% and 19% could generate a 227- and 171-aa GHRH-R, respectively. In addition to structure differences reported, the renal GHRH-R transcribed exhibited a long 5′ UTR (405 nt) contrarily to that of the pituitary receptor which is very short (27 nt).

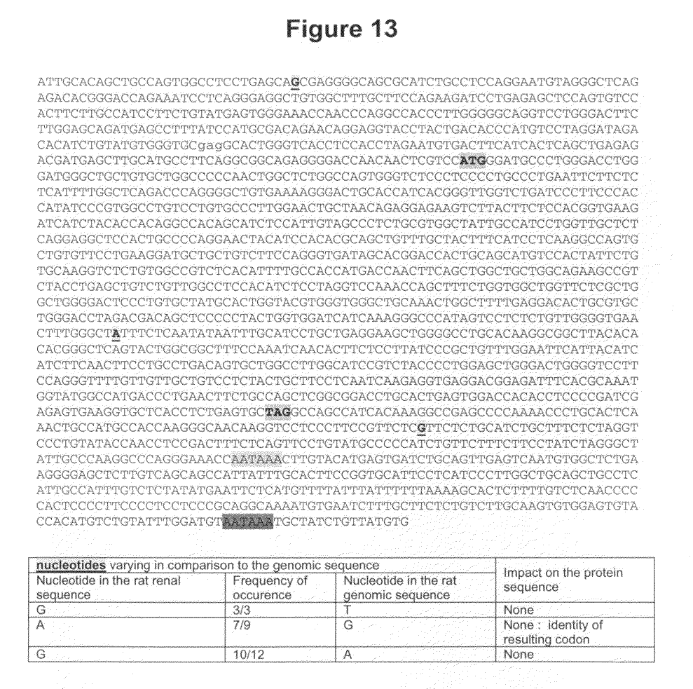

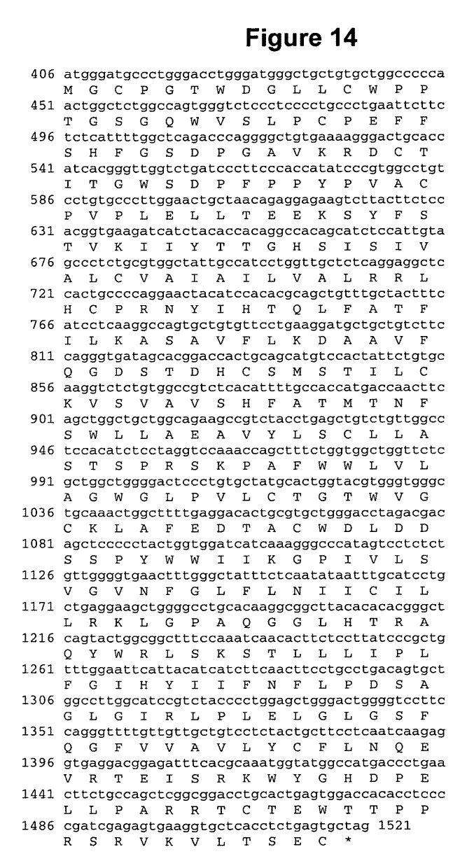

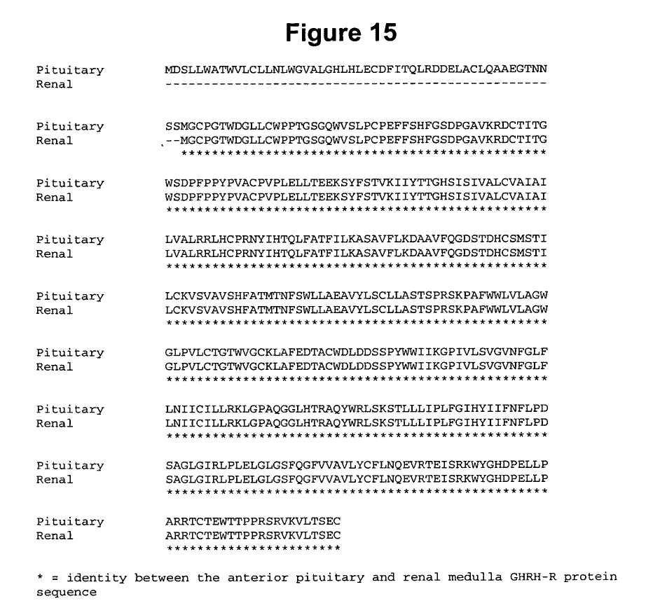

The majority of the GHRH-R cDNA sequences found in renal medulla led to a unique amino acid sequence identical to the portion 53-423 of the 423-aa rat anterior pituitary GHRH-R. The nucleic acid sequence of rat renal medulla GHRH receptor is shown in FIG. 13 and its deduced protein sequence is found in FIG. 14. A comparison of the GHRH-R protein sequence between anterior pituitary and renal medulla is found in FIG. 15.

Detection of Renal GHRH-R RT-PCR Products in Purified Rat Thin Limb Henle's Loop Cells and Anterior Pituitary

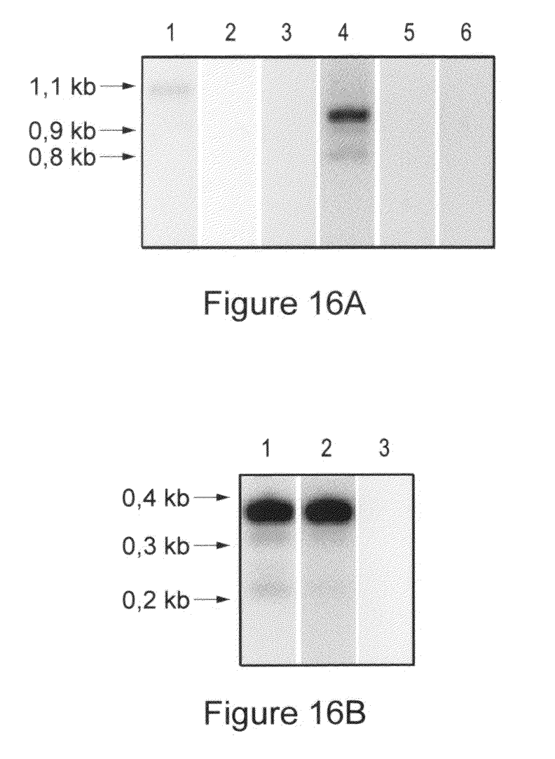

The presence of exons 1M and 2M was validated in tlHL cells of the renal medulla as this cell type contains the highest level of GHRH-R in renal tissue and anterior pituitary. PCR amplification of total RNA from tlHL cells was performed using primers targeting exon 1M and 11M (exon 10 in the pituitary) or exon 2M and 11M (Table 2, FIG. 16A). The size of PCR products obtained using primer pairs Ex-1M/Ex-11M (FIG. 16A, lane 1) and Ex-2M/Ex-11M (FIG. 16A, lane 4) was of 1.10 and 0.97 kb, respectively (expected size: 1.10 and 0.94 kb). No specific amplification was observed with anterior pituitary (FIG. 16A, lanes 2, 5) and liver (FIG. 16A, lanes 3, 6) total RNA. Therefore, the novel cDNA sequences identified in the renal medulla GHRH-R, with exons 1M and 2M shows a tissue-selectivity. The presence of a 0.8-kb product (FIG. 16, lane 4) when the primer pair Ex 2M/Ex 11M was used could be compatible with the presence of a cDNA lacking exon 8M (expected size: 0.79 kb). When the primer pair Ex 6P/Ex 11M was used, a strong signal was obtained (0.40 kb; expected size: 0.37 kb) both with renal (FIG. 16B, lane 1) and pituitary RNA (FIG. 16B, lane 2), indicating that the presence of an entity lacking exon 8M (exon 7 in the pituitary) is likely very low in tlHL and absent in anterior pituitary. No PCR product was detected in liver RNA with primers Ex 6P/Ex 11M (FIG. 16B, lane 3).

Characterization of the Renal Medulla and Anterior Pituitary 5′ Upstream Untranscribed Region

Using Transcription Element Search System (TESS) tools, regions 16601-18600 and 36080-38079 upstream to the transcription initiation site of the renal and anterior pituitary GHRH-R, respectively, were analyzed. As shown in Tables 5 and 6, putative binding sites for several known transcription factors were identified. The most abundant number of sites for transcription factors, found both in the anterior pituitary and renal 5′ untranscribed GHRH-R region (Table 5), were: glucocorticoid receptor (GR), Pit 1 (POU1F1a), upstream stimulatory factors 1 and 2 (USF and USF2), and Brain-2 (POU3F2). The most abundant and specific binding sites to the renal 5′ untranscribed GHRH-R region, was F2F (Table 6). Most of putative pituitary transcription binding sites found in 2 kb upstream the anterior pituitary GHRH-R cDNA transcription start site, were also present in this 2-kb cDNA renal medulla sequence, such as CREB (cAMP response element binding-protein), GR (glucocorticoid receptor), POU1F1a (Pit-1) and T3R-alpha (thyroid hormone receptor). Among common binding sites, Pit-1 and GR was well known to stimulate GHRH-R expression in pituitary. But in renal medulla this same regulator won't lead to pituitary GHRH-R expression. This situation suggests that GHRH-R pituitary promoter is not accessible to the binding of this transcription factor, on tlHL cells. In addition, several binding sites are unique to GHRH-R renal upstream region compared to GHRH-R pituitary upstream region. These different factors may be responsible to the GHRH-R tissue-specific expression in renal medulla. The most abundant binding site was found for the F2F transcription factor. F2F are involved principally in gene repression in non pituitary cells. In kidney, F2F may repress GHRH-R expression in non tlHL cells to keep GHRH-R expression specific to tlHL.

TABLE 5
Putative transcription factor binding sites found in the 5′
untranscribed region of renal medulla GHRH-R and anterior pituitary

| | Number of sites | Number of sites |
|---|---|---|
| | in 5′untranscribed | in 5′-untranscribed |
| | region of the renal | region of the |
| | medulla GHRH-R | anterior pituitary |
| | (region 16601- | GHRH-R (region |
| Name | 18600) | 36080-38079) |
| Glucocorticoid receptor | 19 | 18 |
| (GR) | | |
| Pit-1 (POU1F1a) | 10 | 3 |
| Upstream stimulatory | 9 | 3 |
| factors (USF) | | |
| Upstream stimulatory | 7 | 3 |
| factors-2 (USF2) | | |
| Brain-2 (POU3F2) | 7 | 3 |
| CCAAT/enhancer binding | 6 | 4 |
| protein α (C/EBP α) | | |
| Pan-2(E12) | 4 | 7 |
| Interleukin-6 response | 4 | 7 |
| element-binding protein | | |
| (IL-6 RE-BP) | | |
| Thyroïd hormone receptor | 4 | 4 |
| a (T3R-α) | | |
| c-Jun | 4 | 2 |
| Enhancer-binding protein- | 4 | 2 |
| 45 (EBP-45) | | |
| PEA3 | 3 | 3 |
| c-Fos | 3 | 2 |
| GATA-4 | 3 | 2 |
| Nuclear factor-1 (NF1) | 3 | 2 |
| Nuclear factor BA1 | 3 | 1 |
| (NF-BA1) | | |
| Hepatocyte nuclear factor | 2 | 3 |
| α (HNF-1) | | |
| Myogenin | 2 | 3 |
| Pancreatic transcription | 2 | 3 |
| factor 1 (PTF-1) | | |
| δ-Crystallin enhancer | 2 | 2 |
| factor 1 (δEF1) | | |
| Nerve growth factor | 2 | 2 |
| induced-B (NGFI-B_{—} | | |
| Peroxisome proliferator- | 2 | 2 |
| activated receptor α | | |
| (PPAR-α) | | |
| Snail | 2 | 2 |
| cAMP response element | 2 | 1 |
| binding-protein (CREB) | | |
| Neuronal stem cell | 2 | 1 |
| leukemia (NSCL-1) | | |
| Specificity protein 1 | 1 | 3 |
| (Sp1) | | |
| Activator Protein-1 | 1 | 2 |
| (AP-1) | | |
| Androgen receptor (AR) | 1 | 2 |
| Cone rod homeobox | 1 | 2 |
| protein | | |
| (crx) | | |
| Hepatocyte nuclear factor | 1 | 2 |
| 3 (HNF-3α) | | |
| Hepatocyte nuclear factor- | 1 | 1 |
| 3B (HNF-3B) | | |
| Hepatocyte nuclear factor- | 1 | 1 |
| 3γ (HNF-3γ) | | |

TABLE 6
List of putative transcription factor binding sites specific to the
5′-untranscribed region of renal medulla or anterior pituitary GHRH-R

| | | Number of sites in 5′un- |
|---|---|---|
| | | transcribed region of the |
| | | renal medulla GHRH-R |
| | Name | (region 16601-18600) |
| Example | (F2F) | 7 |
| 11 | Transcription factor-IID | 3 |
| Identificat | (TFIID) | |
| | c-Myc | 2 |
| | Hepatocyte nuclear factor- | 2 |
| | 1B (HNF-1B) | |
| | Aryl-hydrocarbon receptor | 1 |
| | nuclear translocator (Arnt) | |
| | basic leucine zipper (bZIP) | 1 |
| | c-Myb | 1 |
| | chicken ovalbumin upstream | 1 |
| | promoter-I (COUP) | |
| | Delta CREB; CREB-A | 1 |
| | (variant d'épissage | |
| | de CREB) | |
| | Enhancer-binding protein-40 | 1 |
| | (EBP-40) | |
| | GA binding protein | 1 |
| | GABP | 1 |
| | Homeobox | 1 |
| | lymphoid enhancing factor | 1 |
| | 1 (LEF-1) | |
| | Activating transcription | 1 |
| | factor 3 (ATF-3) | |
| | Mammalian achaete-scute | 1 |
| | homolog 1 (MASH-1) | |
| | Mammalian achaete-scute | 1 |
| | homolog 1 (MASH-2) | |
| | Muscle regulatory factor-4 | 1 |
| | (MRF4) | |
| | Mycn | 1 |
| | | Number of sites in 5′un- |
| | | transcribed region of the |
| | | anterior pituitary GHRH-R |
| | Name | (region 36080-38079) |
| | Sox5 | 2 |
| | Zinc finger-42_1-4 | 2 |
| | (ZNF42_1-4) | |
| | CCAAT-binding factor (CBP) | 1 |
| | D-binding protein (DBP) | 1 |
| | Nuclear factor-1/L | 1 |
| | (NF-1/L) | |
| | sis-inducing factor (SIF) | 1 |
| | translation elongation | 1 |
| | factor-2 (TEF2) | |

Identification of the 5′ and 3′ cDNA sequence of the GHRH-R from human renal medullawas performed using the same strategy used for the rat GHRH-R(SMART RACE cDNA Amplification kit (Clontech). Poly (A) RNA from human renal medulla and pituitary was used along with the following primer sequences: B1, 3′ RACE primer: Positions 546 to 573 of the human GHRH-R sequence in Genbank (AY557192) B_{1: 5}′-GGA TGC TGC CCT TTT CCA CAG CGA CGA C-3′; B2, 5′ RACE primer: Positions 684 to 706 of the human GHRH-R sequence. B₂: 5′-GGG AGG TGG AGG CCA GGA GGC AG-3′; C1, 3′ Nested RACE primer: Positions 570 to 597 of the human GHRH-R sequence in Genbank (AY557192) C₁: 5′-CGA CAC TGA CCA CTG CAG CTT CTC CAC T-3′; C2, 5′ Nested RACE primer: Positions 671 to 697 of the human GHRH-R sequence. C₂: 5′-AGG CCA GGA GGC AGT TCA GGT AGA CGG-3′.

Amplified sequences in the 5′ and 3′ RACE strategies overlaped each other in a tract of 161 nucleotides to facilitate sequence analysis. To document the relative abundance of human renal GHRH-R variants, RT-PCR was performed using several specific primers from different exons. The post-mortem renal medullas and pituitaries were obtained from two different men, dead from massive heart attack in their fifties (Quebec-Transplant, Montreal via Dr J Tremblay and the University of St-Jacques de Compostela, Spain via Dr T Garcia-Caballero).

Two main sequences encoding respectively a 400 amino acids (SEQ ID NO.: 16) and a 416 amino acids (SEQ ID NO.: 17) protein were identified, with the 400 amino acids sequence being the most abundant. The 400 amino acids protein corresponds to amino acids 24 to 425 of human pituitary GHRH receptor. The 416 amino acids protein corresponds to amino acids 54-423 of human pituitary GHRH receptor with the first 46 amino acids (N-terminus) being unique to the human renal form of GHRH receptor.

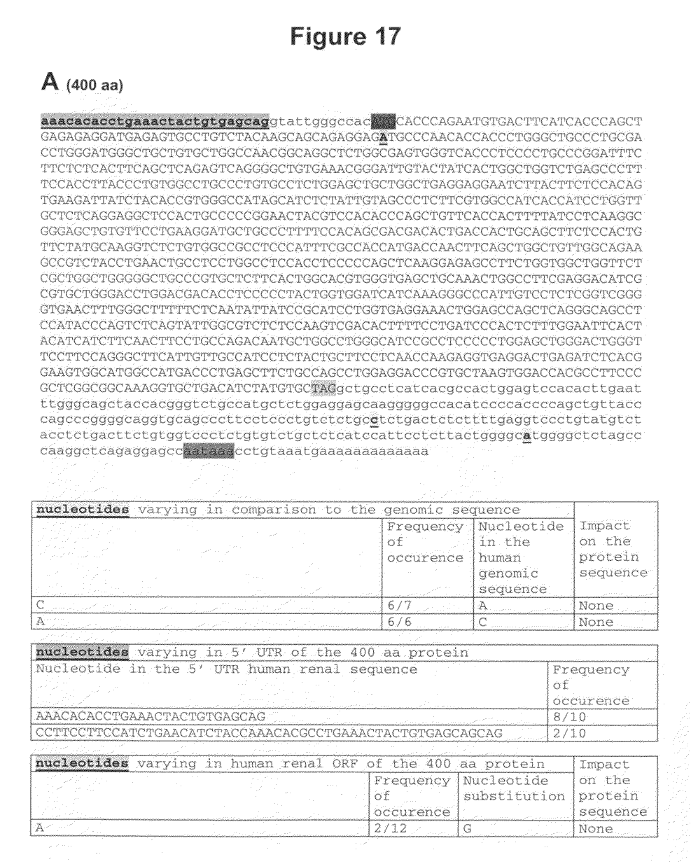

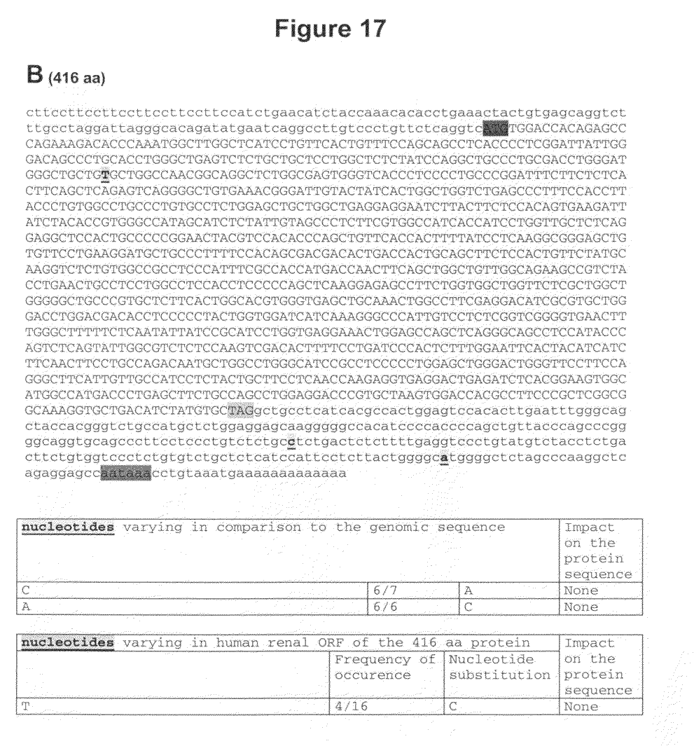

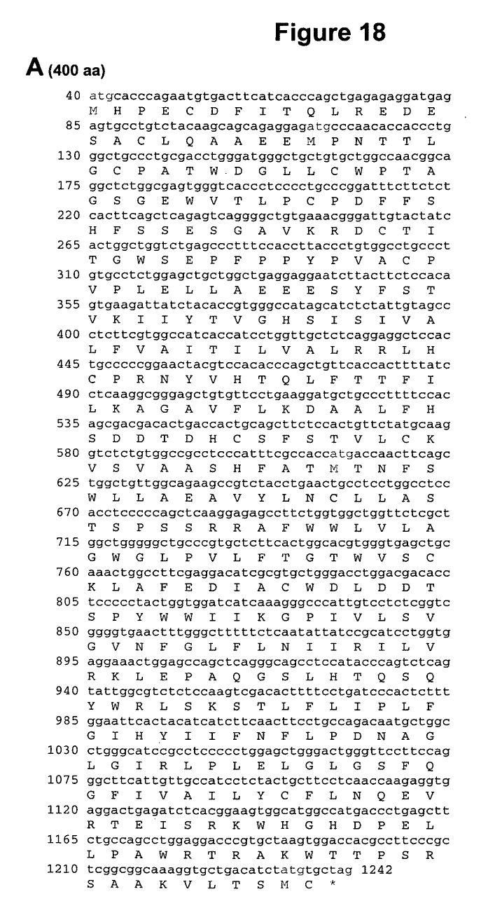

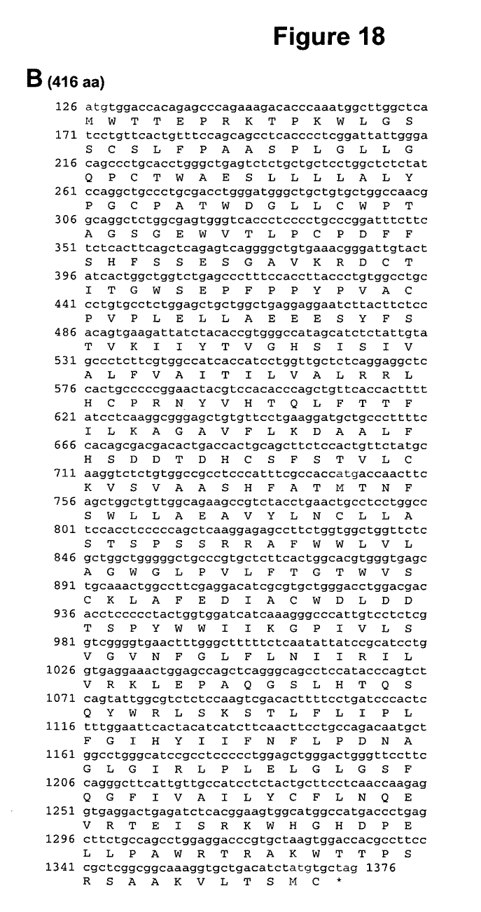

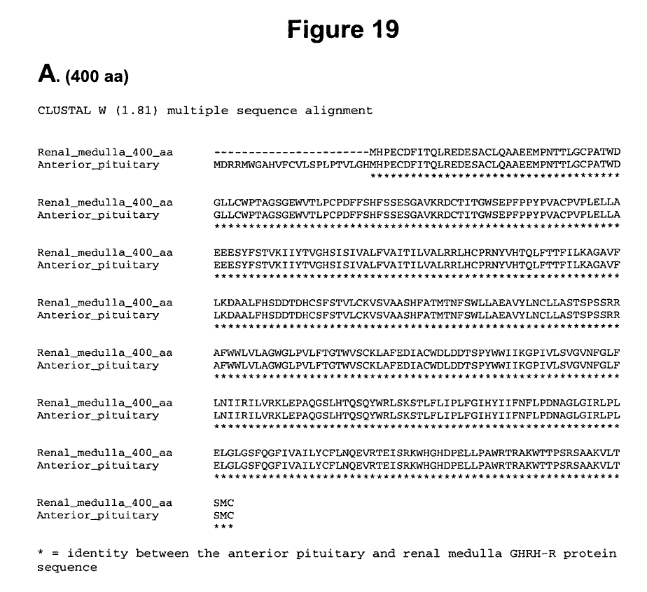

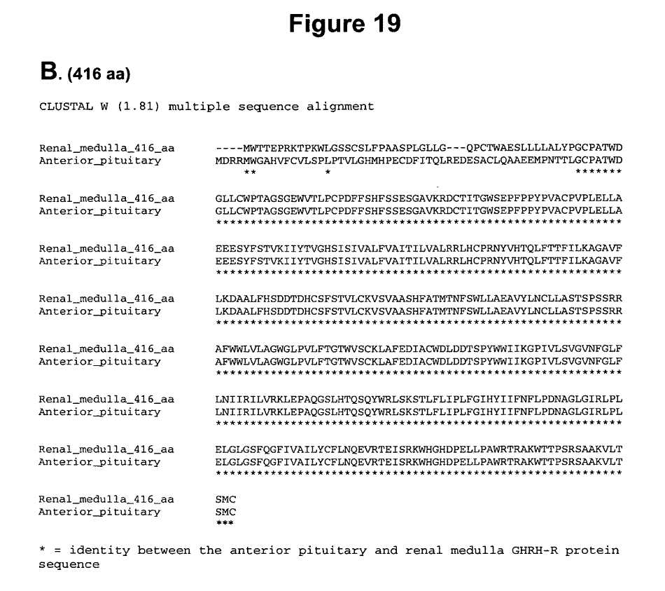

The nucleic acid sequence of human renal medulla GHRH receptor is shown in FIG. 17 and its deduced protein sequence is found in FIG. 18. A comparison of the GHRH-R protein sequence between anterior pituitary and renal medulla is found in FIG. 19.

The position of exons of the human anterior pituitary and human renal GHRH-R on chromosome 7 genomic sequence is shown in Table 7.

TABLE 7
Position of exons of the human anterior pituitary
and renal GHRH-R on chromosome 7 genomic sequence

| | Anterior pituitary | Renal medulla | Renal medulla |
|---|---|---|---|
| | sequence | sequence (400 aa) | sequence (416 aa) |
| Exon 1 | 30970209-30970265 | 30974797-30974848 | — |
| Exon 2 | 30974974-30975076 | 30974971-30975076 | 30974782-30974973 |
| Exon 3 | 30975203-30975310 | 30975203-30975310 | 30975131-30975310 |
| Exon 4 | 30976007-30976104 | 30976007-30976104 | 30976007-30976104 |
| Exon 5 | 30977259-30977356 | 30977259-30977356 | 30977259-30977356 |
| Exon 6 | 30978103-30978235 | 30978103-30978235 | 30978103-30978236 |
| Exon 7 | 30980125-30980278 | 30980125-30980278 | 30980126-30980278 |
| Exon 8 | 30980569-30980629 | 30980569-30980629 | 30980569-30980629 |
| Exon 9 | 30981111-30981180 | 30981111-30981180 | 30981111-30981180 |
| Exon 10 | 30981917-30982008 | 30981917-30982008 | 30981917-30982008 |
| Exon 11 | 30982569-30982698 | 30982569-30982698 | 30982569-30982698 |
| Exon 12 | 30983421-30983462 | 30983421-30983462 | 30983421-30983462 |
| Exon 13 | 30985259-30985384 | 30985259-30985668 | 30985259-30985668 |

In the present study, regulation of renal and pituitary GHRH-R mRNA levels was examined using in vivo models of NaCl or water homeostasis disruption. The presence of a unique GHRH/GHRH-R system in Henle's loop ascending thin limb cell and the specific regulation of GHRH-R mRNA levels and GHRH sensitivity in a situation of hyperosmotic stress, together with the strong effect of GHRH on mitochondrial and nuclear DNA repair/synthesis, indicate a role for GHRH in renoprotection. GHRH appears to be involved in adaptive processes related to DNA repair and/or synthesis thereby protecting ascending limb cell function in subjects with renal vulnerability (aging, diabetes) and were a health event could lead to a production of oxidative stress (antibiotic toxicity, cancer chemotherapeutic agent toxicity, infection, inflammation, ischemia) and renal failure. The Applicant has come to the unexpected discovery that the GHRH receptor (GHRH-R) expressed in kidney cells is different than the pituitary GHRH receptor. The renal GHRH-R is biologically active even if it does not contain the first 52 amino acids and/or the first 80 amino acids of the pituitary GHRH-R.

Although the present invention has been described by way of exemplary embodiments, it should be understood by those skilled in the art that the foregoing and various other changes, omission and additions may be made therein and thereto, without departing from the spirit and scope of the present invention as defined in the appended claims.

SEQUENCE LISTING
SEQ ID NO.: 1-
GHRH(1-44)
Tyr Ala Asp Ala Ile Phe Thr Asn Ser Tyr Arg Lys Val Leu Gly Gln Leu Ser Ala Arg Lys
Leu Leu Gln Asp Ile Met Ser Arg Gln Gln Gly Glu Ser Asn Gln Glu Arg Gly Ala Arg Ala
Arg Leu
SEQ ID NO. 2:
Tyr Ala Asp Ala Ile Phe Thr Asn Ser Tyr Arg Lys Val Leu Gly Gln Leu Ser Ala Arg Lys
Leu Leu Gln Asp Ile Met Ser Arg-Xaa
Wherein Xaa is either absent or any amino acid sequence of 1 up to 15 residues
SEQ ID NO.: 3-
GHRH(1-29)
Tyr Ala Asp Ala Ile Phe Thr Asn Ser Tyr Arg Lys Val Leu Gly Gln Leu Ser Ala Arg Lys
Leu Leu Gln Asp Ile Met Ser Arg
SEQ ID NO.: 4
Tyr-D-Ala²-Asp-Ala-Ile-Phe-Thr-Ala-Ser-Tyr-Arg-Lys-Val-Leu-Ala-Gln-Leu-Ser-Ala-Arg-
Lys-Lys-Leu-Gln-Asp-Ile-Met-Ser-Arg-Xaa,
wherein Xaa is either absent or any amino acid sequence of 1 up to 15 residues
SEQ ID NO.: 5
Tyr-D-Ala²-Asp-Ala-Ile-Phe-Thr-Asn-Ser-D-Tyr¹⁰-Arg-Lys-Val-Leu-Gly-Gln-Leu-Ser-Ala-
Arg-Lys-Lys-Leu-Gln-Asp-Ile-Met-Ser-Arg-Xaa
wherein Xaa is either absent or any amino acid sequence of 1 up to 15 residues
SEQ ID NO.: 6
Tyr- D-Ala²-Asp-Ala-Ile-Phe-Thr-Asn-Ser-D-Tyr¹⁰-Arg-Lys-Val-Leu-D-Ala¹⁵-Gln-Leu-Ser-
Ala-Arg-Lys-Lys²²-Leu-Gln-Asp-Ile-Met-Ser-Arg-Xaa
wherein Xaa is either absent or any amino acid sequence of 1 up to 15 residues
SEQ ID NO.: 7
Tyr Ala Asp Ala Ile Phe Thr Ala⁸ Ser Tyr Arg Lys Val Leu Ala¹⁵ Gln Leu Ser Ala Arg Lys
Ala²² Leu Gln Asp Ile Met Ser Arg
SEQ ID NO.: 8
Tyr Ala Asp Ala Ile Phe Thr Ala⁸ Ala⁹ Tyr Arg Lys Val Leu Ala¹⁵ Gln Leu Ser Ala Arg Lys
Ala²² Leu Gln Asp Ile Met Ser Arg
SEQ ID NO.: 9
Tyr Ala Asp Ala Ile Phe Thr Asn Ser Tyr Arg Lys Val Leu Gly Gln Leu Ser Ala Arg Lys
Lys²² Leu Gln Asp Ile Met Ser Arg
SEQ ID NO.: 10
Tyr-Xaa2-Asp-Ala-Ile-Phe-Thr-Xaa8-Xaa9-Xaa10-Arg-Lys-Val-Leu-Xaa15-Gln-Leu-Ser-
Ala-Arg-Xaa21-Xaa22-Leu-Gln-Asp-Ile-Met-Ser-Arg-Xaa30,
wherein:
Xaa2 is Ala or D-Ala;
Xaa 8 is Asn, D-Asn or Ala;
Xaa 9 is Ser or Ala;
Xaa 10 is Tyr or D-Tyr;
Xaa 15 is Gly, Ala or D-Ala;
Xaa 21 is Lys or D-Lys;
Xaa 22 is Leu, D-Leu, Lys or Ala; and
Xaa 30 is a bond or any amino acid sequence of 1 up to 15 residues and wherein the
analogue comprises at least one of the above amino acid substitution in comparison with
the amino acid sequence of the native form of hGHRH1-29.
SEQ ID NO.: 11
<220>
<221> misc_feature
<222> (31) . . . (31)
<223> nucleotide varying in comparison to the genomic sequence
<220>
<221> misc_feature
<222> (1146) . . . (1146)
<223> nucleotide varying in comparison to the genomic sequence
<220>
<221> misc_feature
<222> (1608) . . . (1608)
<223> nucleotide varying in comparison to the genomic sequence
<400> 1

| | |
|---|---|
| attgcacagc tgccagtggc ctcctgagca gcgaggggca gcgcatctgc ctccaggaat | 60 |
| gtagggctca gagacacggg accagaaatc ctcagggagg ctgtggcttt gcttccagaa | 120 |
| gatcctgaga gctccagtgt ccacttcttg ccatccttct gtatgagtgg gaaaccaacc | 180 |
| caggccaccc ttgggggcag gtcctgggac ttcttggagc agatgagcct ttatccatgc | 240 |
| gacagaacag gaggtaccta ctgacaccca tgtcctagga tagacacatc tgtatgtggg | 300 |
| tgcgaggcac tgggtcacct ccacctagaa tgtgacttca tcactcagct gagagacgat | 360 |
| gagcttgcat gccttcaggc ggcagagggg accaacaact cgtccatggg atgccctggg | 420 |
| acctgggatg ggctgctgtg ctggccccca actggctctg gccagtgggt ctccctcccc | 480 |
| tgccctgaat tcttctctca ttttggctca gacccagggg ctgtgaaaag ggactgcacc | 540 |
| atcacgggtt ggtctgatcc cttcccacca tatcccgtgg cctgtcctgt gcccttggaa | 600 |
| ctgctaacag aggagaagtc ttacttctcc acggtgaaga tcatctacac cacaggccac | 660 |
| agcatctcca ttgtagccct ctgcgtggct attgccatcc tggttgctct caggaggctc | 720 |
| cactgcccca ggaactacat ccacacgcag ctgtttgcta ctttcatcct caaggccagt | 780 |
| gctgtgttcc tgaaggatgc tgctgtcttc cagggtgata gcacggacca ctgcagcatg | 840 |
| tccactattc tgtgcaaggt ctctgtggcc gtctcacatt ttgccaccat gaccaacttc | 900 |
| agctggctgc tggcagaagc cgtctacctg agctgtctgt tggcctccac atctcctagg | 960 |
| tccaaaccag ctttctggtg gctggttctc gctggctggg gactccctgt gctatgcact | 1020 |
| ggtacgtggg tgggctgcaa actggctttt gaggacactg cgtgctggga cctagacgac | 1080 |
| agctccccct actggtggat catcaaaggg cccatagtcc tctctgttgg ggtgaacttt | 1140 |
| gggctatttc tcaatataat ttgcatcctg ctgaggaagc tggggcctgc acaaggcggc | 1200 |
| ttacacacac gggctcagta ctggcggctt tccaaatcaa cacttctcct tatcccgctg | 1260 |
| tttggaattc attacatcat cttcaacttc ctgcctgaca gtgctggcct tggcatccgt | 1320 |
| ctacccctgg agctgggact ggggtccttc cagggttttg ttgttgctgt cctctactgc | 1380 |
| ttcctcaatc aagaggtgag gacggagatt tcacgcaaat ggtatggcca tgaccctgaa | 1440 |
| cttctgccag ctcggcggac ctgcactgag tggaccacac ctccccgatc gagagtgaag | 1500 |
| gtgctcacct ctgagtgcta ggccagccat cacaaaggcc gagccccaaa accctgcact | 1560 |
| caaactgcca tgccaccaag ggcaacaagg tcctcccttc cgttctcgtt ctctgcatct | 1620 |
| gctttctcta ggtccctgta taccaacctc cgactttctc agttcctgta tgcccccatc | 1680 |
| tgttctttct tcctatctag ggctattgcc caaggcccag ggaaaccaat aaacttgtac | 1740 |
| atgagtgatc tgcagttgag tcaatgtggc tctgaagggg agctcttgtc agcagccatt | 1800 |
| atttgcactt ccggtgcatt cctcatccct tggctgcagc tgcctcattg ccatttgtct | 1860 |
| ctatatgaat tctcatgttt tatttatttt ttaaaagcac tcttttgtct caacccccac | 1920 |
| tccccttccc ctcctcccgc aggcaaaatg tgaatctttg cttctctgtc ttgcaagtgt | 1980 |
| ggagtgtacc acatgtctgt atttggatgt aataaatgct atctgttatg tg | 2032 |

SEQ ID NO.: 12

| | |
|---|---|
| atgggatgcc ctgggacctg ggatgggctg ctgtgctggc ccccaactgg ctctggccag | 60 |
| tgggtctccc tcccctgccc tgaattcttc tctcattttg gctcagaccc aggggctgtg | 120 |
| aaaagggact gcaccatcac gggttggtct gatcccttcc caccatatcc cgtggcctgt | 180 |
| cctgtgccct tggaactgct aacagaggag aagtcttact tctccacggt gaagatcatc | 240 |
| tacaccacag gccacagcat ctccattgta gccctctgcg tggctattgc catcctggtt | 300 |
| gctctcagga ggctccactg ccccaggaac tacatccaca cgcagctgtt tgctactttc | 360 |
| atcctcaagg ccagtgctgt gttcctgaag gatgctgctg tcttccaggg tgatagcacg | 420 |
| gaccactgca gcatgtccac tattctgtgc aaggtctctg tggccgtctc acattttgcc | 480 |
| accatgacca acttcagctg gctgctggca gaagccgtct acctgagctg tctgttggcc | 540 |
| tccacatctc ctaggtccaa accagctttc tggtggctgg ttctcgctgg ctggggactc | 600 |
| cctgtgctat gcactggtac gtgggtgggc tgcaaactgg cttttgagga cactgcgtgc | 660 |
| tgggacctag acgacagctc cccctactgg tggatcatca aagggcccat agtcctctct | 720 |
| gttggggtga actttgggct atttctcaat ataatttgca tcctgctgag gaagctgggg | 780 |
| cctgcacaag gcggcttaca cacacgggct cagtactggc ggctttccaa atcaacactt | 840 |
| ctccttatcc cgctgtttgg aattcattac atcatcttca acttcctgcc tgacagtgct | 900 |
| ggccttggca tccgtctacc cctggagctg ggactggggt ccttccaggg ttttgttgtt | 960 |
| gctgtcctct actgcttcct caatcaagag gtgaggacgg agatttcacg caaatggtat | 1020 |
| ggccatgacc ctgaacttct gccagctcgg cggacctgca ctgagtggac cacacctccc | 1080 |
| cgatcgagag tgaaggtgct cacctctgag tgctag | 1116 |

```
SEQ ID NO.: 13
Met Gly Cys Pro Gly Thr Trp Asp Gly Leu Leu Cys Trp Pro Pro Thr
1 5 10 15
Gly Ser Gly Gln Trp Val Ser Leu Pro Cys Pro Glu Phe Phe Ser His
                        20 25 30
Phe Gly Ser Asp Pro Gly Ala Val Lys Arg Asp Cys Thr Ile Thr Gly
                35 40 45
Trp Ser Asp Pro Phe Pro Pro Tyr Pro Val Ala Cys Pro Val Pro Leu
        50 55 60
Glu Leu Leu Thr Glu Glu Lys Ser Tyr Phe Ser Thr Val Lys Ile Ile
65 70 75 80
Tyr Thr Thr Gly His Ser Ile Ser Ile Val Ala Leu Cys Val Ala Ile
                                85 90 95
Ala Ile Leu Val Ala Leu Arg Arg Leu His Cys Pro Arg Asn Tyr Ile
                        100 105 110
His Thr Gln Leu Phe Ala Thr Phe Ile Leu Lys Ala Ser Ala Val Phe
                115 120 125
Leu Lys Asp Ala Ala Val Phe Gln Gly Asp Ser Thr Asp His Cys Ser
        130 135 140
Met Ser Thr Ile Leu Cys Lys Val Ser Val Ala Val Ser His Phe Ala
145 150 155 160
Thr Met Thr Asn Phe Ser Trp Leu Leu Ala Glu Ala Val Tyr Leu Ser
                                165 170 175
Cys Leu Leu Ala Ser Thr Ser Pro Arg Ser Lys Pro Ala Phe Trp Trp
                        180 185 190
Leu Val Leu Ala Gly Trp Gly Leu Pro Val Leu Cys Thr Gly Thr Trp
                195 200 205
Val Gly Cys Lys Leu Ala Phe Glu Asp Thr Ala Cys Trp Asp Leu Asp
        210 215 220
Asp Ser Ser Pro Tyr Trp Trp Ile Ile Lys Gly Pro Ile Val Leu Ser
225 230 235 240
Val Gly Val Asn Phe Gly Leu Phe Leu Asn Ile Ile Cys Ile Leu Leu
                                245 250 255
Arg Lys Leu Gly Pro Ala Gln Gly Gly Leu His Thr Arg Ala Gln Tyr
                        260 265 270
Trp Arg Leu Ser Lys Ser Thr Leu Leu Leu Ile Pro Leu Phe Gly Ile
                275 280 285
His Tyr Ile Ile Phe Asn Phe Leu Pro Asp Ser Ala Gly Leu Gly Ile
        290 295 300
Arg Leu Pro Leu Glu Leu Gly Leu Gly Ser Phe Gln Gly Phe Val Val
305 310 315 320
Ala Val Leu Tyr Cys Phe Leu Asn Gln Glu Val Arg Thr Glu Ile Ser
                        325 330 335
Arg Lys Trp Tyr Gly His Asp Pro Glu Leu Leu Pro Ala Arg Arg Thr
                340 345 350
Cys Thr Glu Trp Thr Thr Pro Pro Arg Ser Arg Val Lys Val Leu Thr
        355 360 365
Ser Glu Cys
370
SEQ ID NO.: 14
52 first amino acids of AAD26335.1 or NP_036982.1 (Rattus norvegicus)
MDSLLWATWV LCLLNLWGVA LGHLHLECDF ITQLRDDELA CLQAAEGTNN SS
SEQ ID NO.: 15
80 first amino acids of EDL88088.1 or EDL88086.1 (Rattus norvegicus)
MQLGGIARDS LGGKGHGSLG RTTGTTAMDS LLWATWVLCL LNLWGVALGH
LHLECDFITQ LRDDELACLQ AAEGTNNSS
SEQ ID NO.: 16
Human renal GHRH-R (400aa)
M H P E C D F I T Q L R E D E S A C L Q A A E E M P N T T L
G C P A T W D G L L C W P T A G S G E W V T L P C P D F F S
H F S S E S G A V K R D C T I T G W S E P F P P Y P V A C P
V P L E L L A E E E S Y F S T V K I I Y T V G H S I S I V A
L F V A I T I L V A L R R L H C P R N Y V H T Q L F T T F I
L K A G A V F L K D A A L F H S D D T D H C S F S T V L C K
V S V A A S H F A T M T N F S W L L A E A V Y L N C L L A S
T S P S S R R A F W W L V L A G W G L P V L F T G T W V S C
K L A F E D I A C W D L D D T S P Y W W I I K G P I V L S V
G V N F G L F L N I I R I L V R K L E P A Q G S L H T Q S Q
Y W R L S K S T L F L I P L F G I H Y I I F N F L P D N A G
L G I R L P L E L G L G S F Q G F I V A I L Y C F L N Q E V
R T E I S R K W H G H D P E L L P A W R T R A K W T T P S R
S A A K V L T S M C
SEQ ID NO.: 17
Human renal GHRH-R (416aa)
M W T T E P R K T P K W L G S S C S L F P A A S P L G L L G
Q P C T W A E S L L L L A L Y P G C P A T W D G L L C W P T
A G S G E W V T L P C P D F F S H F S S E S G A V K R D C T
I T G W S E P F P P Y P V A C P V P L E L L A E E E S Y F S
T V K I I Y T V G H S I S I V A L F V A I T I L V A L R R L
H C P R N Y V H T Q L F T T F I L K A G A V F L K D A A L F
H S D D T D H C S F S T V L C K V S V A A S H F A T M T N F
S W L L A E A V Y L N C L L A S T S P S S R R A F W W L V L
A G W G L P V L F T G T W V S C K L A F E D I A C W D L D D
T S P Y W W I I K G P I V L S V G V N F G L F L N I I R I L
V R K L E P A Q G S L H T Q S Q Y W R L S K S T L F L I P L
F G I H Y I I F N F L P D N A G L G I R L P L E L G L G S F
Q G F I V A I L Y C F L N Q E V R T E I S R K W H G H D P E
L L P A W R T R A K W T T P S R S A A K V L T S M C
SEQ ID NO.: 18
Human GHRH-R 400 total nuc
aaacacacctgaaactactgtgagcaggtattgggccacatgcacccagaatgtgacttcatcacccagctgagagagg
atgagagtgcctgtctacaagcagcagaggagatgcccaacaccaccctgggctgccctgcgacctgggatgggctgct
gtgctggccaacggcaggctctggcgagtgggtcaccctcccctgcccggatttcttctctcacttcagctcagagtcaggg
gctgtgaaacgggattgtactatcactggctggtctgagccctttccaccttaccctgtggcctgccctgtgcctctggagctgc
tggctgaggaggaatcttacttctccacagtgaagattatctacaccgtgggccatagcatctctattgtagccctcttcgtggc
catcaccatcctggttgctctcaggaggctccactgcccccggaactacgtccacacccagctgttcaccacttttatcctcaa
ggcgggagctgtgttcctgaaggatgctgcccttttccacagcgacgacactgaccactgcagcttctccactgttctatgca
aggtctctgtggccgcctcccatttcgccaccatgaccaacttcagctggctgttggcagaagccgtctacctgaactgcctc
ctggcctccacctcccccagctcaaggagagccttctggtggctggttctcgctggctgggggctgcccgtgctcttcactgg
cacgtgggtgagctgcaaactggccttcgaggacatcgcgtgctgggacctggacgacacctccccctactggtggatcat
caaagggcccattgtcctctcggtcggggtgaactttgggctttttctcaatattatccgcatcctggtgaggaaactggagcc
agctcagggcagcctccatacccagtctcagtattggcgtctctccaagtcgacacttttcctgatcccactctttggaattcact
acatcatcttcaacttcctgccagacaatgctggcctgggcatccgcctccccctggagctgggactgggttccttccagggc
ttcattgttgccatcctctactgcttcctcaaccaagaggtgaggactgagatctcacggaagtggcatggccatgaccctga
gcttctgccagcctggaggacccgtgctaagtggaccacgccttcccgctcggcggcaaaggtgctgacatctatgtgcta
ggctgcctcatcacgccactggagtccacacttgaatttgggcagctaccacgggtctgccatgctctggaggagcaaggg
ggccacatccccaccccagctgttacccagcccggggcaggtgcagcccttcctccctgtctctgcctctgactctcttttgag
gtccctgtatgtctacctctgacttctgtggtccctctgtgtctgctctcatccattcctcttactggggcatggggctctagcccaa
ggctcagaggagccaataaacctgtaaatgaaaaaaaaaaaaa
SEQ ID NO.: 19
Human GHRH-R 400 start nuc
atgcacccagaatgtgacttcatcacccagctgagagaggatgagagtgcctgtctacaagcagcagaggagatgccca
acaccaccctgggctgccctgcgacctgggatgggctgctgtgctggccaacggcaggctctggcgagtgggtcaccctc
ccctgcccggatttcttctctcacttcagctcagagtcaggggctgtgaaacgggattgtactatcactggctggtctgagccct
ttccaccttaccctgtggcctgccctgtgcctctggagctgctggctgaggaggaatcttacttctccacagtgaagattatcta
caccgtgggccatagcatctctattgtagccctcttcgtggccatcaccatcctggttgctctcaggaggctccactgcccccg
gaactacgtccacacccagctgttcaccacttttatcctcaaggcgggagctgtgttcctgaaggatgctgcccttttccacag
cgacgacactgaccactgcagcttctccactgttctatgcaaggtctctgtggccgcctcccatttcgccaccatgaccaactt
cagctggctgttggcagaagccgtctacctgaactgcctcctggcctccacctcccccagctcaaggagagccttctggtgg
ctggttctcgctggctgggggctgcccgtgctcttcactggcacgtgggtgagctgcaaactggccttcgaggacatcgcgtg
ctgggacctggacgacacctccccctactggtggatcatcaaagggcccattgtcctctcggtcggggtgaactttgggctttt
tctcaatattatccgcatcctggtgaggaaactggagccagctcagggcagcctccatacccagtctcagtattggcgtctct
ccaagtcgacacttttcctgatcccactctttggaattcactacatcatcttcaacttcctgccagacaatgctggcctgggcatc
cgcctccccctggagctgggactgggttccttccagggcttcattgttgccatcctctactgcttcctcaaccaagaggtgagg
actgagatctcacggaagtggcatggccatgaccctgagcttctgccagcctggaggacccgtgctaagtggaccacgcc
ttcccgctcggcggcaaaggtgctgacatctatgtgctag
SEQ ID NO.: 20
Human GHRH-R 416 total nuc
cttccttccttccttccttccttccatctgaacatctaccaaacacacctgaaactactgtgagcaggtctttgcctaggattaggg
cacagatatgaatcaggccttgtccctgttctcaggtcatgtggaccacagagcccagaaagacacccaaatggcttggct
catcctgttcactgtttccagcagcctcacccctcggattattgggacagccctgcacctgggctgagtctctgctgctcctggc
tctctatccaggctgccctgcgacctgggatgggctgctgtgctggccaacggcaggctctggcgagtgggtcaccctcccc
tgcccggatttcttctctcacttcagctcagagtcaggggctgtgaaacgggattgtactatcactggctggtctgagccctttcc
accttaccctgtggcctgccctgtgcctctggagctgctggctgaggaggaatcttacttctccacagtgaagattatctacac
cgtgggccatagcatctctattgtagccctcttcgtggccatcaccatcctggttgctctcaggaggctccactgcccccggaa
ctacgtccacacccagctgttcaccacttttatcctcaaggcgggagctgtgttcctgaaggatgctgcccttttccacagcga
cgacactgaccactgcagcttctccactgttctatgcaaggtctctgtggccgcctcccatttcgccaccatgaccaacttcag
ctggctgttggcagaagccgtctacctgaactgcctcctggcctccacctcccccagctcaaggagagccttctggtggctg
gttctcgctggctgggggctgcccgtgctcttcactggcacgtgggtgagctgcaaactggccttcgaggacatcgcgtgctg
ggacctggacgacacctccccctactggtggatcatcaaagggcccattgtcctctcggtcggggtgaactttgggctttttct
caatattatccgcatcctggtgaggaaactggagccagctcagggcagcctccatacccagtctcagtattggcgtctctcc
aagtcgacacttttcctgatcccactctttggaattcactacatcatcttcaacttcctgccagacaatgctggcctgggcatccg
cctccccctggagctgggactgggttccttccagggcttcattgttgccatcctctactgcttcctcaaccaagaggtgaggact
gagatctcacggaagtggcatggccatgaccctgagcttctgccagcctggaggacccgtgctaagtggaccacgccttc
ccgctcggcggcaaaggtgctgacatctatgtgctaggctgcctcatcacgccactggagtccacacttgaatttgggcagc
taccacgggtctgccatgctctggaggagcaagggggccacatccccaccccagctgttacccagcccggggcaggtgc
agcccttcctccctgtctctgcctctgactctcttttgaggtccctgtatgtctacctctgacttctgtggtccctctgtgtctgctctcat
ccattcctcttactggggcatggggctctagcccaaggctcagaggagccaataaacctgtaaatgaaaaaaaaaaaaa
SEQ ID NO.: 21
Human GHRH-R 416 start nuc
atgtggaccacagagcccagaaagacacccaaatggcttggctcatcctgttcactgttccagcagcctcacccctcgga
ttattgggacagccctgcacctgggctgagtctctgctgctcctggctctctatccaggctgccctgcgacctgggatgggctg
ctgtgctggccaacggcaggctctggcgagtgggtcaccctcccctgcccggatttcttctctcacttcagctcagagtcagg
ggctgtgaaacgggattgtactatcactggctggtctgagccctttccaccttaccctgtggcctgccctgtgcctctggagctg
ctggctgaggaggaatcttacttctccacagtgaagattatctacaccgtgggccatagcatctctattgtagccctcttcgtgg
ccatcaccatcctggttgctctcaggaggctccactgcccccggaactacgtccacacccagctgttcaccacttttatcctca
aggcgggagctgtgttcctgaaggatgctgcccttttccacagcgacgacactgaccactgcagcttctccactgttctatgc
aaggtctctgtggccgcctcccatttcgccaccatgaccaacttcagctggctgttggcagaagccgtctacctgaactgcct
cctggcctccacctcccccagctcaaggagagccttctggtggctggttctcgctggctgggggctgcccgtgctcttcactg
gcacgtgggtgagctgcaaactggccttcgaggacatcgcgtgctgggacctggacgacacctccccctactggtggatc
atcaaagggcccattgtcctctcggtcggggtgaactttgggctttttctcaatattatccgcatcctggtgaggaaactggagc
cagctcagggcagcctccatacccagtctcagtattggcgtctctccaagtcgacacttttcctgatcccactctttggaattca
ctacatcatcttcaacttcctgccagacaatgctggcctgggcatccgcctccccctggagctgggactgggttccttccagg
gcttcattgttgccatcctctactgcttcctcaaccaagaggtgaggactgagatctcacggaagtggcatggccatgaccct
gagcttctgccagcctggaggacccgtgctaagtggaccacgccttcccgctcggcggcaaaggtgctgacatctatgtgc
tag
SEQ ID NO.: 22
23 first amino acids of growth hormone-releasing hormone receptor Homo
sapiens AAS59864 gi: 45386101
mdrrmwgahv fcvlsplptv lgh
SEQ ID NO.: 23
53 first amino acids of growth hormone-releasing hormone receptor Homo
sapiens AAS59864 gi: 45386101
mdrrmwgahv fcvlsplptv lghmhpecdf itqlredesa clqaaeempn ttl
```

### REFERENCES

1. Lin C, Lin S C, Chang C P et al. Pit-1 dependent expression of the receptor for growth hormone-releasing factor mediates pituitary cell growth. Nature 1992; 360:765-768.
2. Mayo K E. 1992 Molecular cloning and expression of a pituitary specific receptor for growth hormone-releasing factor. Mol Endocrinol 1992; 6:1734-1744.
3. Hsiung H M, Smith D P, Zhang X Y et al. Structure and functional expression of a complementary DNA for porcine growth hormone-releasing hormone receptor. Neuropeptides 1993; 25:1-10.
4. Horikawa R, Gaylinn B D, Lyons Jr C E et al. Molecular cloning of ovine and bovine growth hormone-releasing hormone receptors: the ovine receptor is C-terminally truncated. Endocrinology 2001; 142:2660-2668.
5. Hashimoto K, Koga M, Motomura T et al. Identification of alternatively spliced messenger ribonucleic acid encoding truncated growth hormone-releasing hormone receptor in human pituitary adenomas. J Clin Endocrinol Metab 1995; 80:2933-2939.
6. Tang J, Lagacé G, Castagné J et al. Identification of human growth hormone-releasing hormone receptor splicing variants. J Clin Endocrinol Metab 1995; 80:2381-2387.
7. Gaylinn B D, Harrison J K, Zysk J R et al. Molecular cloning and expression of human anterior pituitary receptor for growth hormone-releasing hormone. Mol Endocrinol 1993; 7:77-84.
8. Toogood A A, Harvey S, Thorner M O et al. Cloning of the chicken pituitary receptor for growth hormone-releasing hormone. Endocrinology 2006; 147:1838-1846.
9. Lee L T, Sju F K, Lau I T et al. Discovery of growth hormone-releasing hormones and receptors in nonmammalian vertebrates. Proc Natl Acad Sci USA 2007; 104:2133-2138.
10. Girard N, Boulanger L, Denis S et al. Differential in vivo regulation of the pituitary growth hormone-releasing hormone (GHRH) receptor by GHRH in young and aged rats. Endocrinology 1999; 140:2836-2842.
11. Miller T L, Godfrey P A, Dealmeida V I et al. The rat growth hormone-releasing hormone receptor gene: structure, regulation, and generation of receptor isoforms with different signaling properties. Endocrinology 1999; 140:4152-4165.
12. Barinaga M, Bilezikjian L M, Vale W W et al. Independent effects of growth hormone releasing factor on growth hormone release and gene transcription. Nature 1985; 314:279-281.
13. Tannenbaum G S, Ling N. The interrelationship of growth hormone (GH)-releasing factor and somatostatin in generation of the ultradian rhythm of GH secretion. Endocrinology 1984; 115:1952-1957.
14. Bilezikjian L M, Vale W W. Stimulation of adenosine 3′,5′-monophosphate production by growth hormone-releasing factor and its inhibition by somatostatin in anterior pituitary cells in vitro. Endocrinology 1983; 113:1726-1731.
15. Cuttler L, Glaum S R, Collins B A et al. Calcium signaling in single growth hormone-releasing factor-responsive pituitary cells. Endocrinology 1992; 130:945-953.
16. Lussier B T, French M B, Moore B C et al. Free intracellular Ca2+ concentration ([Ca2+]i) and growth hormone release from purified rat somatotrophs. I. GH-releasing factor-induced Ca2+influx raises [Ca2+]i. Endocrinology 1991; 128:570-582.
17. Lussier B T, French M B, Moore B C et al. Free intracellular Ca2+ concentration and growth hormone (GH) release from purified rat somototrophs. III. Mechanism of action of GH-releasing factor and somatostatin. Endocrinology 1991; 128:592-603.
18. Chen C, Xu R, Clarke I J et al. Diverse intracellular signaling systems used by growth hormone-releasing hormone in regulating voltage-gated Ca2+ or K channels in pituitary somatotropes. Immunol Cell Biol 2000; 78: 356-368.
19. Billestrup N, Swanson L W, Vale W. Growth hormone-releasing factor stimulates proliferation of somatotrophs in vitro. Proc Natl Acad Sci USA 19986; 83:6854-6857.
20. Dean C E, Porter T E. Regulation of somatotroph differentiation and growth hormone (GH) secretion by corticosterone and GH-releasing hormone during embryonic development. Endocrinology 1999; 140:1104-1110.
21. Godfrey P, Rahal J O, Beamer W G et al. GHRH receptor of little mice contains a missence mutation in the extracellular domain that disrupts receptor function. Nat Genet. 1993; 4:227-232.
22. Lin S C, Lin C R, Gukovsky I. Molecular basis of the little mouse phenotype and implication for cell type-specific growth. Nature 1993; 364:208-213.
23. Pombo C M, Zalvide J, Gaylinn B D et al. Growth hormone-releasing hormone stimulates mitogen-activated protein kinase. Endocrinology 2000; 141:2113-2119.
24. Zeitler P, Siriwardana G. Stimulation of mitogen-activated protein kinase pathway in rat somatotrophs by growth hormone-releasing hormone. Endocrine 2000; 12:257-264.
25. Dickson P R, Feifel D, Vaccarino F J. Blockade of endogenous GRF at dark onset selectively suppresses protein intake. Peptides 1995; 16:7-9.
26. Zhang J, Obal F Jr, Zheng T et al. Intrapreoptic microinjection of GHRH or its antagonist alters sleep in rats. J Neurosci 1999; 19:2187-2194.
27. Guarcello V, Weigent D A, Blalock J E. Growth hormone-releasing hormone receptors on thymocytes and splenocytes from rats. Cell Immunol 1991; 136:291-302.
28. Gallego R, Pintos E, Garcia-Caballero T et al. Cellular distribution of growth hormone-releasing hormone receptor in human reproductive system and breast and prostate cancers. Histol Histopathol 2005; 20:697-706.
29. Kotani E, Usuki S, Kubo T. Effect of growth hormone-releasing hormone on luteinizing hormone stimulated progestin biosynthesis in cultured rat ovarian granulosa cells. Gynecol Endocrinol. 1998; 12:307-313.
30. Moretti C, Bagnato A, Solan N er al. Receptor-mediated actions of growth hormone-releasing factor on granulosa cell differentiation. Endocrinology 1990; 127:2117-2126.
31. Margioris A N, Brockmann G, Bohler H C et al. Expression and localization of growth hormone-releasing hormone messenger ribonucleic acid in rat placenta: in vitro secretion and regulation of its peptide product. Endocrinology 1990; 126:151-158.
32. Ciampani T, Fabbri A, Isidori A et al. Growth hormone-releasing hormone is produced by rat Leydig cell in culture and acts as a positive regulator of Leydig cell function. Endocrinology 1992; 131:2785-2792.
33. Boisvert C, Paré C, Veyrat-Durebex C et al. Localization and regulation of a functional GHRH receptor in the rat renal medulla. Endocrinology 2002; 143:1475-1484.
34. Boulanger L, Girard N, Strecko J et al. Characterization of a growth hormone-releasing hormone binding site in the rat renal medulla. Peptides 2002; 23:1187-1194.
35. Ying W Z, Sanders P W. Dietary salt enhances glomerular endothelial nitric oxide synthase through TGF-beta1. Am J Physiol 1998; 275:F18-F24.
36. Ying W Z, Sanders P W. Dietary salt regulates expression of Tamm-Horsfall glycoprotein in rats. Kidney Int 1998; 54:1150-1156.
37. Shin S J, Wen J D, Chen I H et al. Increased renal ANP synthesis, but decreased or unchanged cardiac ANP synthesis in water-deprived and salt-restricted rats. Kidney Int 1998; 54:1617-1625.
38. Korytko A l, Zeitler P, Cuttler L. Developmental regulation of pituitary growth hormone-releasing hormone receptor gene expression in the rat. Endocrinology 1996; 137:1326-1331.
39. Matsubara S, Sato M, Mizobuchi M et al. Differential gene expression of growth hormone (GH)-releasing hormone (GRH) and GRH receptor in various rat tissues. Endocrinology 1995; 136:4147-4150.
40. Fujinaka Y, Yokogoshi Y, Zhang C Y et al. Tissue-specific molecular heterogeneity of human growth hormone-releasing hormone receptor protein. FEBS Lett 1996; 9394:1-4.
41. Nielsen S, Pallone T, Smith B L et al. Aquaporin-1 water channels in short and long loop descending thin limbs and in descending vasa recta in rat kidney. Am J Physiol 1995; 268:F1023-1037
42. Uchida S, Sasaki S, Nitta K et al. Localization and functional characterization of rat kidney-specific chloride channel, CIC-K1. J Clin Invest 1995; 95:104-113.
43. Kondo Y, Kudo K, Igarashi Y. Functions of ascending thin limb of Henle's loop with special emphasis on mechanism of NaCl transport. Tohoku J Exp Med 1992; 166:75-84.
44. Rodriguez-Iturbe B, Sepassi L, Quiroz Y. Association of mitochondrial SOD deficiency with salt-sensitive hypertension and accelerated renal senescence. J Appl Physiol 2007; 102:255-260.
45. Taylor N E, Cowley A W. Effect of renal medullary H2O2 on salt-induced hypertension and renal injury. Am J Physiol Regul Integr Comp Physiol 2005; 289:R1573-R1579.
46. Liang M, Yuan B, Rute E et al. Insights into Dahl salt-sensitive hypertension revealed by temporal patterns of renal medullary gene expression. Physiol Genomics 2003; 12:229-237.
47. Hisaki R, Fujita H, Saito F. Tempol attenuates the development of hypertensive renal injury in Dahl salt-sensitive rats. Am J Hypertens 2005; 18:707-713.
48. Zhang J J, Bledsoe G, Kato K. Tissue kallikrein attenuates salt-induced renal fibrosis by inhibition of oxidative stress. Kidney Int 2004; 66:722-732.
49. Lee H, Wei Y H Oxidative stress, mitochondrial DNA mutation and apoptosis in aging. Exp Biol Med 232:592-606. Tan A L, Forbes J M, Copper M E 2007 AGE, RAGE, and ROS in diabetic nephropathy. Semin Nephrol 2007; 27:130-143.
50. Favier A 2006 Oxidative stress in human diseases Ann Pharm Fr 2006; 64:390-396.
51. Csiszar A, Toth J, Petri-Peterdi J, Ungvari Z The aging kidney: role of endothelial oxidative stress and inflammation. Acta Physiol Hung 2007; 94:107-115.
52. Morel G, Gallego R, Boulanger L et al. Restricted presence of the growth hormone-releasing hormone receptor to somatotrophs in rat and human pituitaries. Neuroendocrinology 1999; 70:128-136.
53. Bruno F H, Sing J, Berelowitz M. Regulation of rat hypothalamic preprogrowth hormone-releasing factor messenger ribonucleic acid by dietary protein. Endocrinology 1991; 129:1226-1232.
54. Zeitler P, Stevens P, Siriwardana G. Functional GHRH receptor carboxyl terminal isoforms in normal and dwarf (dw) rats. J Mol Endocrinol 1988; 21:363-371.
55. Grupp C, Lottermoser J, Cohen D I et al. Transformation of rat inner medullary fibroblasts to myofibroblasts in vitro. Kidney Int 1997; 52:1279-1290.
56. Gaudreau P, Boulanger L, Abribat T. Affinity of human growth hormone-releasing factor (1-29)NH₂ analogues for GRF binding sites in rat adenopituitary. J Med. Chem. 1992; 35:1864-1869.
57. Trinh-Trang-Tan M M, Bouby N, Coutaud C et al. Quick isolation of rat medullary ascending limbs. Pflügers Arch 1986; 407:228-234.
58. Vetrat-Durebex C, Pomerleau L, Langlois D et al. Internalization and trafficking of the human and rat growth hormone-releasing hormone receptor. J Cell Physiol 2005; 203:335-344.
59. Bradford M. A Rapid and sensitive method for the quantitation of microgram quantities of protein utilizing the principle of protein-dye binding. Anal Biochem 1976; 72:248-254.
60. Mirescu et al., Global assessment of serum antioxidant status in hemodialysis patients. J. Nephrol. 2005; 18:599-605.
61. Agarwal R and Chase S D. Rapid, fluorometric-liquid chromatographic determination of malondialdehyde in biological samples. J of Chromatography B 2002; 775: 121-126.
62. Aebi H, Catalase in vitro, Methods Enzymol 1984; 105:121-6.
63. Paglia D E, Valetine W N., Studies on the quantitative and qualitative characterization of erythrocyte glutathione peroxidase. J. Lab Clin. Invest. 1976; 70:158-69
64. Carlberg I. and B. Mannervik, Glutathione reductase, Methods Enzymol. 13 (1985), pp. 484-499.
65. Arthur J. R. Functional indicators of iodine and selenium status. Proc Nutr Soc. 1999; 58:507-12.
66. Reznick A Z, Packer L. Oxidative damage to proteins: spectrophotometric method for carbonyl assay. Methods Enzymol, 1994; 233:357-63.

1. A method for protecting or treating a mammal against oxidative renal damage, the method comprising the step of administering an effective amount of a ligand to GHRH renal receptor to the mammal.

2. The method of claim 1, wherein the ligand is GHRH, a biologically active fragment of GHRH or a GHRH agonist thereof.

3. The method of claim 2, wherein the ligand is selected from the group consisting of SEQ ID NO.:1, SEQ ID NO.:2 and SEQ ID NO.:3.

4. The method of claim 2, wherein the ligand is selected from the group consisting of SEQ ID NO.:4, SEQ ID NO.:5, SEQ ID NO.:6, SEQ ID NO.:7, SEQ ID NO.:8, SEQ ID NO.:9 and SEQ ID NO.:10.

5. The method of claim 1 wherein the effective amount is not substantially active against anterior pituitary GHRH receptor.

6. The method of claim 5 wherein the effective amount has a protective effect substantially similar to a subcutaneous 1.0 mg rat GHRH(1-29)NH₂ dose per kilogram of body weight per day or lower, in a Sprague Dawley rat submitted to a high-salt diet.

7. The method of claim 6 wherein the effective amount has a protective effect substantially similar to subcutaneous 0.5 mg rat GHRH(1-29)NH₂ dose per kilogram of body weight per day or lower, in a Sprague Dawley rat submitted to a high-salt diet.

8. The method of claim 1 wherein the oxidative renal damage affects Henle's loop cells.

9. The method of claim 1, wherein the oxidative damage is due to exaggerated renal medullary osmolality.

10. The method of claim 1 comprising identifying the mammal in need by determining the presence of a marker associated with oxidative renal damage.

11. The method of claim 1 wherein the mammal suffers or is susceptible of suffering from a disease selected from the group consisting of aging- and frailty-related nephropathy and renal failure, diabetes insipidus, diabetes type I, diabetes II, renal disease glomerulonephritis, bacterial or viral glomerulonephritides, IgA nephropathy, Henoch-Schonlein Purpura, membranoproliferative glomerulonephritis, membranous nephropathy, Sjogren's syndrome, nephrotic syndrome minimal change disease, focal glomerulosclerosis and related disorders, acute renal failure, acute tubulointerstitial nephritis, pyelonephritis, genitourinary (GU) tract inflammatory disease, pre-clampsia, renal graft rejection, leprosy, reflux nephropathy, nephrolithiasis, genetic renal disease, medullary cystic, medullar sponge, polycystic kidney disease, autosomal dominant polycystic kidney disease, autosomal recessive polycystic kidney disease, tuberous sclerosis, von Hippel-Lindau disease, familial thin-glomerular basement membrane disease, collagen III glomerulopathy, fibronectin glomerulopathy, Alport's syndrome, Fabry's disease, Nail-Patella Syndrome, congenital urologic anomalies, monoclonal gammopathies, multiple myeloma, amyloidosis and related disorders, febrile illness, familial Mediterranean fever, HIV infection, AIDS, inflammatory disease, systemic vasculitides, polyarteritis nodosa, Wegener's granulomatosis, polyarteritis, necrotizing and crescentic glomerulonephritis, polymyositis-dermatomyositis, pancreatitis, rheumatoid arthritis, systemic lupus erythematosus, gout, blood disorders, sickle cell disease, thrombotic thrombocytopenia purpura, hemolytic-uremic syndrome, acute cortical necrosis, renal thromboembolism, trauma and surgery, extensive injury, burns, abdominal and vascular surgery, induction of anesthesia, side effect of drug abuse or use of including those generating renal oxidative stress and toxicity such as antibiotics and cancer chemotherapeutic agents, malignant disease, adenocarcinoma, melanoma, lymphoreticular, multiple myeloma, circulatory disease, myocardial infarction, cardiac failure, peripheral vascular disease, hypertension, coronary heart disease, non-atherosclerotic cardiovascular disease, atherosclerotic cardiovascular disease, skin disease, psoriasis, systemic sclerosis, respiratory disease, chronic obstructive pulmonary disease, obstructive sleep apnea, hypoxia at high altitude or endocrine disease, acromegaly, diabetes mellitus and conditions related to antibiotic toxicity, infection, inflammation and ischemia.

12. The method of claim 1 wherein the mammal is subjected to chronic hemodialysis.

13. A method of preventing the death of kidney cells due to oxidative stress in a mammal in need thereof, the method comprising administering an effective amount of a ligand to GHRH renal receptor to the mammal.

14. A method of promoting regeneration of kidney cells in a mammal in need thereof, the method comprising administering a ligand to the GHRH renal receptor the mammal.

15. An isolated polypeptide comprising
a. SEQ ID NO.: 13, SEQ ID NO.:16 or SEQ ID NO.:17,
b. a SEQ ID NO.13, SEQ ID NO.:16 or SEQ ID NO.:17 fragment or
c. a SEQ ID NO.:13, SEQ ID NO.:16 or SEQ ID NO.:17 analog;
wherein said polypeptide is free of a N-terminal amino acid sequence of a pituitary GHRH receptor.

16. An antibody capable of specific binding to the polypeptide of claim 15.

17. An isolated nucleic acid sequence encoding the polypeptide of claim 15.

18. An isolated nucleic acid selected from the group consisting of
a. a polynucleotide comprising SEQ ID NO.11, SEQ ID NO.12, SEQ ID NO.:18, SEQ ID NO.:19, SEQ ID NO.:20 or SEQ ID NO.:21,
b. a polynucleotide comprising a sequence substantially identical to SEQ ID NO.11, SEQ ID NO.12, SEQ ID NO.:18, SEQ ID NO.:19, SEQ ID NO.:20 or to SEQ ID NO.:21,
c. a polynucleotide comprising a sequence substantially complemetary to a. or b. and;
d. a fragment of any one of a., b. or c.;
wherein the nucleic acid is free of a nucleic acid sequence encoding a N-terminal amino acid sequence of a pituitary GHRH receptor.

19. A vector comprising the nucleic acid sequence of claim 17.

20. A vector comprising the nucleic acid sequence of claim 18.

21. An isolated cell expressing the polypeptide of claim 15.

22. The isolated cell of claim 21 wherein the cell is a renal cell.

23. An assay for identifying a ligand which is capable of specific binding to the polypeptide of claim 15 and not to pituitary GHRH receptor, the assay comprising contacting a test ligand with the polypeptide, measuring binding of the test ligand to the polypeptide and determining the identity of the test ligand.


SEQUENCE LISTING (132 sequences)

SEQ ID NO: 1 - protein, 44 aa, Artificial Sequence
  Chemically Synthesized
YADAIFTNSYRKVLGQLSARKLLQDIMSRQQGESNQERGARARL

SEQ ID NO: 2 - protein, 29 aa, Artificial Sequence
  Chemically Synthesized
YADAIFTNSYRKVLGQLSARKLLQDIMSR

SEQ ID NO: 3 - protein, 30 aa, Artificial Sequence
  Chemically Synthesized
YADAIFTNSYRKVLGQLSARKLLQDIMSRX

SEQ ID NO: 4 - protein, 31 aa, Artificial Sequence
  Chemically Synthesized
YADAIFTNSYRKVLGQLSARKLLQDIMSRXX

SEQ ID NO: 5 - protein, 32 aa, Artificial Sequence
  Chemically Synthesized
YADAIFTNSYRKVLGQLSARKLLQDIMSRXXX

SEQ ID NO: 6 - protein, 33 aa, Artificial Sequence
  Chemically Synthesized
YADAIFTNSYRKVLGQLSARKLLQDIMSRXXXX

SEQ ID NO: 7 - protein, 34 aa, Artificial Sequence
  Chemically Synthesized
YADAIFTNSYRKVLGQLSARKLLQDIMSRXXXXX

SEQ ID NO: 8 - protein, 35 aa, Artificial Sequence
  Chemically Synthesized
YADAIFTNSYRKVLGQLSARKLLQDIMSRXXXXXX

SEQ ID NO: 9 - protein, 36 aa, Artificial Sequence
  Chemically Synthesized
YADAIFTNSYRKVLGQLSARKLLQDIMSRXXXXXXX

SEQ ID NO: 10 - protein, 37 aa, Artificial Sequence
  Chemically Syntheized
YADAIFTNSYRKVLGQLSARKLLQDIMSRXXXXXXXX

SEQ ID NO: 11 - protein, 38 aa, Artificial Sequence
  Chemically Synthesized
YADAIFTNSYRKVLGQLSARKLLQDIMSRXXXXXXXXX

SEQ ID NO: 12 - protein, 39 aa, Artificial Sequence
  Chemically Synthesized
YADAIFTNSYRKVLGQLSARKLLQDIMSRXXXXXXXXXX

SEQ ID NO: 13 - protein, 40 aa, Artificial Sequence
  Chemically Synthesized
YADAIFTNSYRKVLGQLSARKLLQDIMSRXXXXXXXXXXX

SEQ ID NO: 14 - protein, 41 aa, Artificial Sequence
  Chemically Synthesized
YADAIFTNSYRKVLGQLSARKLLQDIMSRXXXXXXXXXXXX

SEQ ID NO: 15 - protein, 42 aa, Artificial Sequence
  Chemically Synthesized
YADAIFTNSYRKVLGQLSARKLLQDIMSRXXXXXXXXXXXXX

SEQ ID NO: 16 - protein, 43 aa, Artificial Sequence
  Chemically Synthesized
YADAIFTNSYRKVLGQLSARKLLQDIMSRXXXXXXXXXXXXXX

SEQ ID NO: 17 - protein, 44 aa, Artificial Sequence
  Chemically Synthesized
YADAIFTNSYRKVLGQLSARKLLQDIMSRXXXXXXXXXXXXXXX

SEQ ID NO: 18 - protein, 29 aa, Artificial Sequence
  Chemically Synthe sized
YADAIFTNSYRKVLGQLSARKLLQDIMSR

SEQ ID NO: 19 - protein, 29 aa, Artificial Sequence
  Chemically Synthesized
YXDAIFTASYRKVLAQLSARKKLQDIMSR

SEQ ID NO: 20 - protein, 30 aa, Artificial Sequence
  Chemically Synthesized
YXDAIFTASYRKVLAQLSARKKLQDIMSRX

SEQ ID NO: 21 - protein, 31 aa, Artificial Sequence
  Chemically Synthesized
YXDAIFTASYRKVLAQLSARKKLQDIMSRXX

SEQ ID NO: 22 - protein, 32 aa, Artificial Sequence
  Chemically Synthesized
YXDAIFTASYRKVLAQLSARKKLQDIMSRXXX

SEQ ID NO: 23 - protein, 33 aa, Artificial Sequence
  Chemically Synthesized
YXDAIFTASYRKVLAQLSARKKLQDIMSRXXXX

SEQ ID NO: 24 - protein, 34 aa, Artificial Sequence
  Chemically Synthesized
YXDAIFTASYRKVLAQLSARKKLQDIMSRXXXXX

SEQ ID NO: 25 - protein, 35 aa, Artificial Sequence
  Chemically Synthesized
YXDAIFTASYRKVLAQLSARKKLQDIMSRXXXXXX

SEQ ID NO: 26 - protein, 36 aa, Artificial Sequence
  Chemically Synthesized
YXDAIFTASYRKVLAQLSARKKLQDIMSRXXXXXXX

SEQ ID NO: 27 - protein, 37 aa, Artificial Sequence
  Chemically Synthesized
YXDAIFTASYRKVLAQLSARKKLQDIMSRXXXXXXXX

SEQ ID NO: 28 - protein, 38 aa, Artificial Sequence
  Chemically Synthesized
YXDAIFTASYRKVLAQLSARKKLQDIMSRXXXXXXXXX

SEQ ID NO: 29 - protein, 39 aa, Artificial Sequence
  Chemically Synthesized
YXDAIFTASYRKVLAQLSARKKLQDIMSRXXXXXXXXXX

SEQ ID NO: 30 - protein, 40 aa, Artificial Sequence
  Chemically Synthesized
YXDAIFTASYRKVLAQLSARKKLQDIMSRXXXXXXXXXXX

SEQ ID NO: 31 - protein, 41 aa, Artificial Sequence
  Chemically Synthesized
YXDAIFTASYRKVLAQLSARKKLQDIMSRXXXXXXXXXXXX

SEQ ID NO: 32 - protein, 42 aa, Artificial Sequence
  Chemically Synthesized
YXDAIFTASYRKVLAQLSARKKLQDIMSRXXXXXXXXXXXXX

SEQ ID NO: 33 - protein, 43 aa, Artificial Sequence
  Chemically Synthesized
YXDAIFTASYRKVLAQLSARKKLQDIMSRXXXXXXXXXXXXXX

SEQ ID NO: 34 - protein, 44 aa, Artificial Sequence
  Chemically Synthesized
YXDAIFTASYRKVLAQLSARKKLQDIMSRXXXXXXXXXXXXXXX

SEQ ID NO: 35 - protein, 29 aa, Artificial Sequence
  Chemically Synthesized
YXDAIFTNSXRKVLGQLSARKKLQDIMSR

SEQ ID NO: 36 - protein, 30 aa, Artificial Sequence
  Chemically Synthesized
YXDAIFTNSXRKVLGQLSARKKLQDIMSRX

SEQ ID NO: 37 - protein, 31 aa, Artificial Sequence
  Chemically Synthesized
YXDAIFTNSXRKVLGQLSARKKLQDIMSRXX

SEQ ID NO: 38 - protein, 32 aa, Artificial Sequence
  Chemically Synthesized
YXDAIFTNSXRKVLGQLSARKKLQDIMSRXXX

SEQ ID NO: 39 - protein, 33 aa, Artificial Sequence
  Chemically Synthesized
YXDAIFTNSXRKVLGQLSARKKLQDIMSRXXXX

SEQ ID NO: 40 - protein, 34 aa, Artificial Sequence
  Chemically Synthesized
YXDAIFTNSXRKVLGQLSARKKLQDIMSRXXXXX

SEQ ID NO: 41 - protein, 35 aa, Artificial Sequence
  Chemically Synthesized
YXDAIFTNSXRKVLGQLSARKKLQDIMSRXXXXXX

SEQ ID NO: 42 - protein, 36 aa, Artificial Sequence
  Chemically Synthesized
YXDAIFTNSXRKVLGQLSARKKLQDIMSRXXXXXXX

SEQ ID NO: 43 - protein, 37 aa, Artificial Sequence
  Chemically Synthesized
YXDAIFTNSXRKVLGQLSARKKLQDIMSRXXXXXXXX

SEQ ID NO: 44 - protein, 38 aa, Artificial Sequence
  Chemically Synthesized
YXDAIFTNSXRKVLGQLSARKKLQDIMSRXXXXXXXXX

SEQ ID NO: 45 - protein, 39 aa, Artificial Sequence
  Chemically Synthesized
YXDAIFTNSXRKVLGQLSARKKLQDIMSRXXXXXXXXXX

SEQ ID NO: 46 - protein, 40 aa, Artificial Sequence
  Chemically Synthesized
YXDAIFTNSXRKVLGQLSARKKLQDIMSRXXXXXXXXXXX

SEQ ID NO: 47 - protein, 41 aa, Artificial Sequence
  Chemically Synthesized
YXDAIFTNSXRKVLGQLSARKKLQDIMSRXXXXXXXXXXXX

SEQ ID NO: 48 - protein, 42 aa, Artificial Sequence
  Chemically Synthesized
YXDAIFTNSXRKVLGQLSARKKLQDIMSRXXXXXXXXXXXXX

SEQ ID NO: 49 - protein, 43 aa, Artificial Sequence
  Chemically Synthesized
YXDAIFTNSXRKVLGQLSARKKLQDIMSRXXXXXXXXXXXXXX

SEQ ID NO: 50 - protein, 44 aa, Artificial Sequence
  Chemically Synthesized
YXDAIFTNSXRKVLGQLSARKKLQDIMSRXXXXXXXXXXXXXXX

SEQ ID NO: 51 - protein, 29 aa, Artificial Sequence
  Chemically Synthesized
YXDAIFTNSXRKVLXQLSARKKLQDIMSR

SEQ ID NO: 52 - protein, 30 aa, Artificial Sequence
  Chemically Synthesized
YXDAIFTNSXRKVLXQLSARKKLQDIMSRX

SEQ ID NO: 53 - protein, 31 aa, Artificial Sequence
  Chemically Synthesized
YXDAIFTNSXRKVLXQLSARKKLQDIMSRXX

SEQ ID NO: 54 - protein, 32 aa, Artificial Sequence
  Chemically Synthesized
YXDAIFTNSXRKVLXQLSARKKLQDIMSRXXX

SEQ ID NO: 55 - protein, 33 aa, Artificial Sequence
  Chemically Synthesized
YXDAIFTNSXRKVLXQLSARKKLQDIMSRXXXX

SEQ ID NO: 56 - protein, 34 aa, Artificial Sequence
  Chemically Synthesized
YXDAIFTNSXRKVLXQLSARKKLQDIMSRXXXXX

SEQ ID NO: 57 - protein, 35 aa, Artificial Sequence
  Chemically Synthesized
YXDAIFTNSXRKVLXQLSARKKLQDIMSRXXXXXX

SEQ ID NO: 58 - protein, 36 aa, Artificial Sequence
  Chemically Synthesized
YXDAIFTNSXRKVLXQLSARKKLQDIMSRXXXXXXX

SEQ ID NO: 59 - protein, 37 aa, Artificial Sequence
  Chemically Synthesized
YXDAIFTNSXRKVLXQLSARKKLQDIMSRXXXXXXXX

SEQ ID NO: 60 - protein, 38 aa, Artificial Sequence
  Chemically Synthesized
YXDAIFTNSXRKVLXQLSARKKLQDIMSRXXXXXXXXX

SEQ ID NO: 61 - protein, 39 aa, Artificial Sequence
  Chemically Synthesized
YXDAIFTNSXRKVLXQLSARKKLQDIMSRXXXXXXXXXX

SEQ ID NO: 62 - protein, 40 aa, Artificial Sequence
  Chemically Synthesized
YXDAIFTNSXRKVLXQLSARKKLQDIMSRXXXXXXXXXXX

SEQ ID NO: 63 - protein, 41 aa, Artificial Sequence
  Chemically Synthesized
YXDAIFTNSXRKVLXQLSARKKLQDIMSRXXXXXXXXXXXX

SEQ ID NO: 64 - protein, 42 aa, Artificial Sequence
  Chemically Synthesized
YXDAIFTNSXRKVLXQLSARKKLQDIMSRXXXXXXXXXXXXX

SEQ ID NO: 65 - protein, 43 aa, Artificial Sequence
  Chemically Synthesized
YXDAIFTNSXRKVLXQLSARKKLQDIMSRXXXXXXXXXXXXXX

SEQ ID NO: 66 - protein, 44 aa, Artificial Sequence
  Chemically Synthesized
YXDAIFTNSXRKVLXQLSARKKLQDIMSRXXXXXXXXXXXXXXX

SEQ ID NO: 67 - protein, 29 aa, Artificial Sequence
  Chemically Synthesized
YADAIFTASYRKVLAQLSARKALQDIMSR

SEQ ID NO: 68 - protein, 29 aa, Artificial Sequence
  Chemically Synthesized
YADAIFTAAYRKVLAQLSARKALQDIMSR

SEQ ID NO: 69 - protein, 29 aa, Artificial Sequence
  Chemically Synthesized
YADAIFTNSYRKVLGQLSARKKLQDIMSR

SEQ ID NO: 70 - protein, 29 aa, Artificial Sequence
  Chemically Synthesized
YXDAIFTXXXRKVLXQLSARXXLQDIMSR

SEQ ID NO: 71 - protein, 30 aa, Artificial Sequence
  Chemically Synthesized
YXDAIFTXXXRKVLXQLSARXXLQDIMSRX

SEQ ID NO: 72 - protein, 31 aa, Artificial Sequence
  Chemically Synthesized
YXDAIFTXXXRKVLXQLSARXXLQDIMSRXX

SEQ ID NO: 73 - protein, 32 aa, Artificial Sequence
  Chemically Synthesized
YXDAIFTXXXRKVLXQLSARXXLQDIMSRXXX

SEQ ID NO: 74 - protein, 33 aa, Artificial Sequence
  Chemically Synthesized
YXDAIFTXXXRKVLXQLSARXXLQDIMSRXXXX

SEQ ID NO: 75 - protein, 34 aa, Artificial Sequence
  Chemically Synthesized
YXDAIFTXXXRKVLXQLSARXXLQDIMSRXXXXX

SEQ ID NO: 76 - protein, 35 aa, Artificial Sequence
  Chemically Synthesized
YXDAIFTXXXRKVLXQLSARXXLQDIMSRXXXXXX

SEQ ID NO: 77 - protein, 36 aa, Artificial Sequence
  Chemically Synthesized
YXDAIFTXXXRKVLXQLSARXXLQDIMSRXXXXXXX

SEQ ID NO: 78 - protein, 37 aa, Artificial Sequence
  Chemically Synthesized
YXDAIFTXXXRKVLXQLSARXXLQDIMSRXXXXXXXX

SEQ ID NO: 79 - protein, 38 aa, Artificial Sequence
  Chemically Synthesized
YXDAIFTXXXRKVLXQLSARXXLQDIMSRXXXXXXXXX

SEQ ID NO: 80 - protein, 39 aa, Artificial Sequence
  Chemically Synthesized
YXDAIFTXXXRKVLXQLSARXXLQDIMSRXXXXXXXXXX

SEQ ID NO: 81 - protein, 40 aa, Artificial Sequence
  Chemically Synthesized
YXDAIFTXXXRKVLXQLSARXXLQDIMSRXXXXXXXXXXX

SEQ ID NO: 82 - protein, 41 aa, Artificial Sequence
  Chemically Synthesized
YXDAIFTXXXRKVLXQLSARXXLQDIMSRXXXXXXXXXXXX

SEQ ID NO: 83 - protein, 42 aa, Artificial Sequence
  Chemically Synthesized
YXDAIFTXXXRKVLXQLSARXXLQDIMSRXXXXXXXXXXXXX

SEQ ID NO: 84 - protein, 43 aa, Artificial Sequence
  Chemically Synthesized
YXDAIFTXXXRKVLXQLSARXXLQDIMSRXXXXXXXXXXXXXX

SEQ ID NO: 85 - protein, 44 aa, Artificial Sequence
  Chemically Synthesized
YXDAIFTXXXRKVLXQLSARXXLQDIMSRXXXXXXXXXXXXXXX

SEQ ID NO: 86 - DNA, 2032 bp, Artificial Sequence
  Chemically Synthesized
attgcacagctgccagtggcctcctgagcagcgaggggcagcgcatctgcctccaggaatgtagggctcagagacacgggaccagaaatcctcagggaggctgtggctttgcttccagaagatcctgagagctccagtgtccacttcttgccatccttctgtatgagtgggaaaccaacccaggccacccttgggggcaggtcctgggacttcttggagcagatgagcctttatccatgcgacagaacaggaggtacctactgacacccatgtcctaggatagacacatctgtatgtgggtgcgaggcactgggtcacctccacctagaatgtgacttcatcactcagctgagagacgatgagcttgcatgccttcaggcggcagaggggaccaacaactcgtccatgggatgccctgggacctgggatgggctgctgtgctggcccccaactggctctggccagtgggtctccctcccctgccctgaattcttctctcattttggctcagacccaggggctgtgaaaagggactgcaccatcacgggttggtctgatcccttcccaccatatcccgtggcctgtcctgtgcccttggaactgctaacagaggagaagtcttacttctccacggtgaagatcatctacaccacaggccacagcatctccattgtagccctctgcgtggctattgccatcctggttgctctcaggaggctccactgccccaggaactacatccacacgcagctgtttgctactttcatcctcaaggccagtgctgtgttcctgaaggatgctgctgtcttccagggtgatagcacggaccactgcagcatgtccactattctgtgcaaggtctctgtggccgtctcacattttgccaccatgaccaacttcagctggctgctggcagaagccgtctacctgagctgtctgttggcctccacatctcctaggtccaaaccagctttctggtggctggttctcgctggctggggactccctgtgctatgcactggtacgtgggtgggctgcaaactggcttttgaggacactgcgtgctgggacctagacgacagctccccctactggtggatcatcaaagggcccatagtcctctctgttggggtgaactttgggctatttctcaatataatttgcatcctgctgaggaagctggggcctgcacaaggcggcttacacacacgggctcagtactggcggctttccaaatcaacacttctccttatcccgctgtttggaattcattacatcatcttcaacttcctgcctgacagtgctggccttggcatccgtctacccctggagctgggactggggtccttccagggttttgttgttgctgtcctctactgcttcctcaatcaagaggtgaggacggagatttcacgcaaatggtatggccatgaccctgaacttctgccagctcggcggacctgcactgagtggaccacacctccccgatcgagagtgaaggtgctcacctctgagtgctaggccagccatcacaaaggccgagccccaaaaccctgcactcaaactgccatgccaccaagggcaacaaggtcctcccttccgttctcgttctctgcatctgctttctctaggtccctgtataccaacctccgactttctcagttcctgtatgcccccatctgttctttcttcctatctagggctattgcccaaggcccagggaaaccaataaacttgtacatgagtgatctgcagttgagtcaatgtggctctgaaggggagctcttgtcagcagccattatttgcacttccggtgcattcctcatcccttggctgcagctgcctcattgccatttgtctctatatgaattctcatgttttatttattttttaaaagcactcttttgtctcaacccccactccccttcccctcctcccgcaggcaaaatgtgaatctttgcttctctgtcttgcaagtgtggagtgtaccacatgtctgtatttggatgtaataaatgctatctgttatgtg

SEQ ID NO: 87 - DNA, 1116 bp, Artificial Sequence
  Chemically Synthesized
atgggatgccctgggacctgggatgggctgctgtgctggcccccaactggctctggccagtgggtctccctcccctgccctgaattcttctctcattttggctcagacccaggggctgtgaaaagggactgcaccatcacgggttggtctgatcccttcccaccatatcccgtggcctgtcctgtgcccttggaactgctaacagaggagaagtcttacttctccacggtgaagatcatctacaccacaggccacagcatctccattgtagccctctgcgtggctattgccatcctggttgctctcaggaggctccactgccccaggaactacatccacacgcagctgtttgctactttcatcctcaaggccagtgctgtgttcctgaaggatgctgctgtcttccagggtgatagcacggaccactgcagcatgtccactattctgtgcaaggtctctgtggccgtctcacattttgccaccatgaccaacttcagctggctgctggcagaagccgtctacctgagctgtctgttggcctccacatctcctaggtccaaaccagctttctggtggctggttctcgctggctggggactccctgtgctatgcactggtacgtgggtgggctgcaaactggcttttgaggacactgcgtgctgggacctagacgacagctccccctactggtggatcatcaaagggcccatagtcctctctgttggggtgaactttgggctatttctcaatataatttgcatcctgctgaggaagctggggcctgcacaaggcggcttacacacacgggctcagtactggcggctttccaaatcaacacttctccttatcccgctgtttggaattcattacatcatcttcaacttcctgcctgacagtgctggccttggcatccgtctacccctggagctgggactggggtccttccagggttttgttgttgctgtcctctactgcttcctcaatcaagaggtgaggacggagatttcacgcaaatggtatggccatgaccctgaacttctgccagctcggcggacctgcactgagtggaccacacctccccgatcgagagtgaaggtgctcacctctgagtgctag

SEQ ID NO: 88 - protein, 371 aa, Artificial Sequence
  Chemically Synthesized
MGCPGTWDGLLCWPPTGSGQWVSLPCPEFFSHFGSDPGAVKRDCTITGWSDPFPPYPVACPVPLELLTEEKSYFSTVKIIYTTGHSISIVALCVAIAILVALRRLHCPRNYIHTQLFATFILKASAVFLKDAAVFQGDSTDHCSMSTILCKVSVAVSHFATMTNFSWLLAEAVYLSCLLASTSPRSKPAFWWLVLAGWGLPVLCTGTWVGCKLAFEDTACWDLDDSSPYWWIIKGPIVLSVGVNFGLFLNIICILLRKLGPAQGGLHTRAQYWRLSKSTLLLIPLFGIHYIIFNFLPDSAGLGIRLPLELGLGSFQGFVVAVLYCFLNQEVRTEISRKWYGHDPELLPARRTCTEWTTPPRSRVKVLTSEC

SEQ ID NO: 89 - protein, 52 aa, Artificial Sequence
  Chemically Synthesized
MDSLLWATWVLCLLNLWGVALGHLHLECDFITQLRDDELACLQAAEGTNNSS

SEQ ID NO: 90 - protein, 79 aa, Artificial Sequence
  Chemically Synthesized
MQLGGIARDSLGGKGHGSLGRTTGTTAMDSLLWATWVLCLLNLWGVALGHLHLECDFITQLRDDELACLQAAEGTNNSS

SEQ ID NO: 91 - protein, 400 aa, Artificial Sequence
  Chemically Synthesized
MHPECDFITQLREDESACLQAAEEMPNTTLGCPATWDGLLCWPTAGSGEWVTLPCPDFFSHFSSESGAVKRDCTITGWSEPFPPYPVACPVPLELLAEEESYFSTVKIIYTVGHSISIVALFVAITILVALRRLHCPRNYVHTQLFTTFILKAGAVFLKDAALFHSDDTDHCSFSTVLCKVSVAASHFATMTNFSWLLAEAVYLNCLLASTSPSSRRAFWWLVLAGWGLPVLFTGTWVSCKLAFEDIACWDLDDTSPYWWIIKGPIVLSVGVNFGLFLNIIRILVRKLEPAQGSLHTQSQYWRLSKSTLFLIPLFGIHYIIFNFLPDNAGLGIRLPLELGLGSFQGFIVAILYCFLNQEVRTEISRKWHGHDPELLPAWRTRAKWTTPSRSAAKVLTSMC

SEQ ID NO: 92 - protein, 416 aa, Artificial Sequence
  Chemically Synthesized
MWTTEPRKTPKWLGSSCSLFPAASPLGLLGQPCTWAESLLLLALYPGCPATWDGLLCWPTAGSGEWVTLPCPDFFSHFSSESGAVKRDCTITGWSEPFPPYPVACPVPLELLAEEESYFSTVKIIYTVGHSISIVALFVAITILVALRRLHCPRNYVHTQLFTTFILKAGAVFLKDAALFHSDDTDHCSFSTVLCKVSVAASHFATMTNFSWLLAEAVYLNCLLASTSPSSRRAFWWLVLAGWGLPVLFTGTWVSCKLAFEDIACWDLDDTSPYWWIIKGPIVLSVGVNFGLFLNIIRILVRKLEPAQGSLHTQSQYWRLSKSTLFLIPLFGIHYIIFNFLPDNAGLGIRLPLELGLGSFQGFIVAILYCFLNQEVRTEISRKWHGHDPELLPAWRTRAKWTTPSRSAAKVLTSMC

SEQ ID NO: 93 - DNA, 1537 bp, Artificial Sequence
  Chemically Synthesized
aaacacacctgaaactactgtgagcaggtattgggccacatgcacccagaatgtgacttcatcacccagctgagagaggatgagagtgcctgtctacaagcagcagaggagatgcccaacaccaccctgggctgccctgcgacctgggatgggctgctgtgctggccaacggcaggctctggcgagtgggtcaccctcccctgcccggatttcttctctcacttcagctcagagtcaggggctgtgaaacgggattgtactatcactggctggtctgagccctttccaccttaccctgtggcctgccctgtgcctctggagctgctggctgaggaggaatcttacttctccacagtgaagattatctacaccgtgggccatagcatctctattgtagccctcttcgtggccatcaccatcctggttgctctcaggaggctccactgcccccggaactacgtccacacccagctgttcaccacttttatcctcaaggcgggagctgtgttcctgaaggatgctgcccttttccacagcgacgacactgaccactgcagcttctccactgttctatgcaaggtctctgtggccgcctcccatttcgccaccatgaccaacttcagctggctgttggcagaagccgtctacctgaactgcctcctggcctccacctcccccagctcaaggagagccttctggtggctggttctcgctggctgggggctgcccgtgctcttcactggcacgtgggtgagctgcaaactggccttcgaggacatcgcgtgctgggacctggacgacacctccccctactggtggatcatcaaagggcccattgtcctctcggtcggggtgaactttgggctttttctcaatattatccgcatcctggtgaggaaactggagccagctcagggcagcctccatacccagtctcagtattggcgtctctccaagtcgacacttttcctgatcccactctttggaattcactacatcatcttcaacttcctgccagacaatgctggcctgggcatccgcctccccctggagctgggactgggttccttccagggcttcattgttgccatcctctactgcttcctcaaccaagaggtgaggactgagatctcacggaagtggcatggccatgaccctgagcttctgccagcctggaggacccgtgctaagtggaccacgccttcccgctcggcggcaaaggtgctgacatctatgtgctaggctgcctcatcacgccactggagtccacacttgaatttgggcagctaccacgggtctgccatgctctggaggagcaagggggccacatccccaccccagctgttacccagcccggggcaggtgcagcccttcctccctgtctctgcctctgactctcttttgaggtccctgtatgtctacctctgacttctgtggtccctctgtgtctgctctcatccattcctcttactggggcatggggctctagcccaaggctcagaggagccaataaacctgtaaatgaaaaaaaaaaaaa

SEQ ID NO: 94 - DNA, 1203 bp, Artificial Sequence
  Chemically Synthesized
atgcacccagaatgtgacttcatcacccagctgagagaggatgagagtgcctgtctacaagcagcagaggagatgcccaacaccaccctgggctgccctgcgacctgggatgggctgctgtgctggccaacggcaggctctggcgagtgggtcaccctcccctgcccggatttcttctctcacttcagctcagagtcaggggctgtgaaacgggattgtactatcactggctggtctgagccctttccaccttaccctgtggcctgccctgtgcctctggagctgctggctgaggaggaatcttacttctccacagtgaagattatctacaccgtgggccatagcatctctattgtagccctcttcgtggccatcaccatcctggttgctctcaggaggctccactgcccccggaactacgtccacacccagctgttcaccacttttatcctcaaggcgggagctgtgttcctgaaggatgctgcccttttccacagcgacgacactgaccactgcagcttctccactgttctatgcaaggtctctgtggccgcctcccatttcgccaccatgaccaacttcagctggctgttggcagaagccgtctacctgaactgcctcctggcctccacctcccccagctcaaggagagccttctggtggctggttctcgctggctgggggctgcccgtgctcttcactggcacgtgggtgagctgcaaactggccttcgaggacatcgcgtgctgggacctggacgacacctccccctactggtggatcatcaaagggcccattgtcctctcggtcggggtgaactttgggctttttctcaatattatccgcatcctggtgaggaaactggagccagctcagggcagcctccatacccagtctcagtattggcgtctctccaagtcgacacttttcctgatcccactctttggaattcactacatcatcttcaacttcctgccagacaatgctggcctgggcatccgcctccccctggagctgggactgggttccttccagggcttcattgttgccatcctctactgcttcctcaaccaagaggtgaggactgagatctcacggaagtggcatggccatgaccctgagcttctgccagcctggaggacccgtgctaagtggaccacgccttcccgctcggcggcaaaggtgctgacatctatgtgctag

SEQ ID NO: 95 - DNA, 1671 bp, Artificial Sequence
  Chemically Synthesized
cttccttccttccttccttccttccatctgaacatctaccaaacacacctgaaactactgtgagcaggtctttgcctaggattagggcacagatatgaatcaggccttgtccctgttctcaggtcatgtggaccacagagcccagaaagacacccaaatggcttggctcatcctgttcactgtttccagcagcctcacccctcggattattgggacagccctgcacctgggctgagtctctgctgctcctggctctctatccaggctgccctgcgacctgggatgggctgctgtgctggccaacggcaggctctggcgagtgggtcaccctcccctgcccggatttcttctctcacttcagctcagagtcaggggctgtgaaacgggattgtactatcactggctggtctgagccctttccaccttaccctgtggcctgccctgtgcctctggagctgctggctgaggaggaatcttacttctccacagtgaagattatctacaccgtgggccatagcatctctattgtagccctcttcgtggccatcaccatcctggttgctctcaggaggctccactgcccccggaactacgtccacacccagctgttcaccacttttatcctcaaggcgggagctgtgttcctgaaggatgctgcccttttccacagcgacgacactgaccactgcagcttctccactgttctatgcaaggtctctgtggccgcctcccatttcgccaccatgaccaacttcagctggctgttggcagaagccgtctacctgaactgcctcctggcctccacctcccccagctcaaggagagccttctggtggctggttctcgctggctgggggctgcccgtgctcttcactggcacgtgggtgagctgcaaactggccttcgaggacatcgcgtgctgggacctggacgacacctccccctactggtggatcatcaaagggcccattgtcctctcggtcggggtgaactttgggctttttctcaatattatccgcatcctggtgaggaaactggagccagctcagggcagcctccatacccagtctcagtattggcgtctctccaagtcgacacttttcctgatcccactctttggaattcactacatcatcttcaacttcctgccagacaatgctggcctgggcatccgcctccccctggagctgggactgggttccttccagggcttcattgttgccatcctctactgcttcctcaaccaagaggtgaggactgagatctcacggaagtggcatggccatgaccctgagcttctgccagcctggaggacccgtgctaagtggaccacgccttcccgctcggcggcaaaggtgctgacatctatgtgctaggctgcctcatcacgccactggagtccacacttgaatttgggcagctaccacgggtctgccatgctctggaggagcaagggggccacatccccaccccagctgttacccagcccggggcaggtgcagcccttcctccctgtctctgcctctgactctcttttgaggtccctgtatgtctacctctgacttctgtggtccctctgtgtctgctctcatccattcctcttactggggcatggggctctagcccaaggctcagaggagccaataaacctgtaaatgaaaaaaaaaaaaa

SEQ ID NO: 96 - DNA, 1251 bp, Artificial Sequence
  Chemically Synthesized
atgtggaccacagagcccagaaagacacccaaatggcttggctcatcctgttcactgtttccagcagcctcacccctcggattattgggacagccctgcacctgggctgagtctctgctgctcctggctctctatccaggctgccctgcgacctgggatgggctgctgtgctggccaacggcaggctctggcgagtgggtcaccctcccctgcccggatttcttctctcacttcagctcagagtcaggggctgtgaaacgggattgtactatcactggctggtctgagccctttccaccttaccctgtggcctgccctgtgcctctggagctgctggctgaggaggaatcttacttctccacagtgaagattatctacaccgtgggccatagcatctctattgtagccctcttcgtggccatcaccatcctggttgctctcaggaggctccactgcccccggaactacgtccacacccagctgttcaccacttttatcctcaaggcgggagctgtgttcctgaaggatgctgcccttttccacagcgacgacactgaccactgcagcttctccactgttctatgcaaggtctctgtggccgcctcccatttcgccaccatgaccaacttcagctggctgttggcagaagccgtctacctgaactgcctcctggcctccacctcccccagctcaaggagagccttctggtggctggttctcgctggctgggggctgcccgtgctcttcactggcacgtgggtgagctgcaaactggccttcgaggacatcgcgtgctgggacctggacgacacctccccctactggtggatcatcaaagggcccattgtcctctcggtcggggtgaactttgggctttttctcaatattatccgcatcctggtgaggaaactggagccagctcagggcagcctccatacccagtctcagtattggcgtctctccaagtcgacacttttcctgatcccactctttggaattcactacatcatcttcaacttcctgccagacaatgctggcctgggcatccgcctccccctggagctgggactgggttccttccagggcttcattgttgccatcctctactgcttcctcaaccaagaggtgaggactgagatctcacggaagtggcatggccatgaccctgagcttctgccagcctggaggacccgtgctaagtggaccacgccttcccgctcggcggcaaaggtgctgacatctatgtgctag

SEQ ID NO: 97 - protein, 23 aa, Artificial Sequence
  Chemically Synthesized
MDRRMWGAHVFCVLSPLPTVLGH

SEQ ID NO: 98 - protein, 53 aa, Artificial Sequence
  Chemically Synthesized
MDRRMWGAHVFCVLSPLPTVLGHMHPECDFITQLREDESACLQAAEEMPNTTL

SEQ ID NO: 99 - DNA, 20 bp, Artificial Sequence
  Chemically Synthesized
gggtgtgaaccacgagaaat

SEQ ID NO: 100 - DNA, 20 bp, Artificial Sequence
  Chemically Synthesized
actgtggtcatgagcccttc

SEQ ID NO: 101 - DNA, 21 bp, Artificial Sequence
  Chemically Synthesized
atgccactctgggtgttcttt

SEQ ID NO: 102 - DNA, 20 bp, Artificial Sequence
  Chemically Synthesized
gcagtttgcgggcatataat

SEQ ID NO: 103 - DNA, 20 bp, Artificial Sequence
  Chemically Synthesized
ctctgcttgctgaacctgtg

SEQ ID NO: 104 - DNA, 20 bp, Artificial Sequence
  Chemically Synthesized
catcccatggacgagttgtt

SEQ ID NO: 105 - DNA, 20 bp, Artificial Sequence
  Chemically Synthesized
ctgctgtcttccagggtgat

SEQ ID NO: 106 - DNA, 20 bp, Artificial Sequence
  Chemically Synthesized
taggagatgtggaggccaac

SEQ ID NO: 107 - DNA, 20 bp, Artificial Sequence
  Chemically Synthesized
acttcctgcctgacagtgct

SEQ ID NO: 108 - DNA, 20 bp, Artificial Sequence
  Chemically Synthesized
tggcagaagttcagggtcat

SEQ ID NO: 109 - DNA, 20 bp, Artificial Sequence
  Chemically Synthesized
ctgctgagctccctaccagt

SEQ ID NO: 110 - DNA, 18 bp, Artificial Sequence
  Chemically Synthesized
cagcccgaggaggagttg

SEQ ID NO: 111 - DNA, 21 bp, Artificial Sequence
  Chemically Synthesized
gcttctccacggttctgtgca

SEQ ID NO: 112 - DNA, 20 bp, Artificial Sequence
  Chemically Synthesized
tgggtgacgtagaggccaag

SEQ ID NO: 113 - DNA, 22 bp, Artificial Sequence
  Chemically Synthesized
gctccttccagggcttcattgt

SEQ ID NO: 114 - DNA, 21 bp, Artificial Sequence
  Chemically Synthesized
gaaggctttgcccatttggca

SEQ ID NO: 115 - DNA, 30 bp, Artificial Sequence
  Chemically Synthesized
aagcagtggtatcaacgcagagtacgcggg

SEQ ID NO: 116 - DNA, 57 bp, Artificial Sequence
  Chemically Synthesized
aagcagtggtatcaacgcagagtacttttttttttttttttttttttttttttttnn

SEQ ID NO: 117 - DNA, 45 bp, Artificial Sequence
  Chemically Synthesized
ctaatacgactcactatagggcaagcagtggtatcaacgcagagt

SEQ ID NO: 118 - DNA, 22 bp, Artificial Sequence
  Chemically Synthesized
ctaatacgactcactatagggc

SEQ ID NO: 119 - DNA, 23 bp, Artificial Sequence
  Chemically Synthesized
aagcagtggtatcaacgcagagt

SEQ ID NO: 120 - DNA, 28 bp, Artificial Sequence
  Chemically Synthesized
cagcccaaagttcaccccaacagagagg

SEQ ID NO: 121 - DNA, 27 bp, Artificial Sequence
  Chemically Synthesized
ccagtagggggagctgtcgtctaggtc

SEQ ID NO: 122 - DNA, 25 bp, Artificial Sequence
  Chemically Synthesized
ctggtggctggttctcgctggctgg

SEQ ID NO: 123 - DNA, 25 bp, Artificial Sequence
  Chemically Synthesized
tctcgctggctggggactccctgtg

SEQ ID NO: 124 - DNA, 19 bp, Artificial Sequence
  Chemically Synthesized
cctgtgctatgcactggta

SEQ ID NO: 125 - DNA, 19 bp, Artificial Sequence
  Chemically Synthesized
agacacgggaccagaaatc

SEQ ID NO: 126 - DNA, 20 bp, Artificial Sequence
  Chemically Synthesized
agcctttatccatgcgacag

SEQ ID NO: 127 - DNA, 24 bp, Artificial Sequence
  Chemically Synthesized
gatgcaaattatattgagaaacag

SEQ ID NO: 128 - DNA, 28 bp, Artificial Sequence
  Chemically Synthesized
ggatgctgcccttttccacagcgacgac

SEQ ID NO: 129 - DNA, 23 bp, Artificial Sequence
  Chemically Synthesized
gggaggtggaggccaggaggcag

SEQ ID NO: 130 - DNA, 28 bp, Artificial Sequence
  Chemically Synthesized
cgacactgaccactgcagcttctccact

SEQ ID NO: 131 - DNA, 27 bp, Artificial Sequence
  Chemically Synthesized
aggccaggaggcagttcaggtagacgg

SEQ ID NO: 132 - DNA, 27 bp, Artificial Sequence
  Chemically Synthesized
tttttttttttttttttttttttttnn